(19) United States
(12) Patent Application Publication
(10) Pub. No.: US 2021/0371480 A1
(43) Pub. Date: Dec. 2, 2021

(54) COMPOSITIONS AND METHODS FOR TREATING AGE-RELATED MACULAR DEGENERATION AND OTHER DISEASES

(71) Applicant: Gemini Therapeutics Inc., Cambridge, MA (US)

(72) Inventors: James McLaughlin, Cambridge, MA (US); Adarsha Koirala, Framingham, MA (US); Lisa Huang, Cambridge, MA (US)

(21) Appl. No.: 17/288,311

(22) PCT Filed: Oct. 23, 2019
(86) PCT No.: PCT/US2019/057686

§ 371 Date: Apr. 23, 2021

Related U.S. Application Data
(60) Provisional application No. 62/749,373, filed on Oct. 23, 2018.

(51) Int. Cl. C07K 14/47; C12N 9/64; C12N 15/86; A61P 27/02

(52) U.S. Cl. CPC C07K 14/472; C12N 9/6424; A61K 48/00; A61P 27/02; C12N 2750/14143; C12N 15/86

(57) ABSTRACT

The present disclosure provides compositions and methods for treating, preventing, or inhibiting diseases of the eye. In one aspect, the disclosure provides recombinant CF1 adeno-associated virus (rAAV) vectors comprising a complement system gene.

### CROSS-REFERENCE TO RELATED APPLICATIONS

This application claims the benefit of priority from U.S. Provisional Application No. 62/749,373, filed Oct. 23, 2018. The specification of the foregoing application is incorporated herein by reference in its entirety.

### BACKGROUND OF THE DISCLOSURE

Age-related macular degeneration (AMD) is a medical condition and is the leading cause of legal blindness in Western societies. AMD typically affects older adults and results in a loss of central vision due to degenerative and neovascular changes to the macula, a pigmented region at the center of the retina which is responsible for visual acuity. There are four major AMD subtypes: Early AMD; Intermediate AMD; Advanced non-neovascular (“Dry”) AMD; and Advanced neovascular (“Wet”) AMD. Typically. AMD is identified by the focal hyperpigmentation of the retinal pigment epithelium (RPE) and accumulation of drusen deposits and/or geographic atrophy. The size and number of drusen deposits or level of geographic atrophy typically correlates with AMD severity.

AMD occurs in up to 8% of individuals over the age of 60, and the prevalence of AMD continues to increase with age. The U.S. is anticipated to have nearly 22 million cases of AMD by the year 2050, while global cases of AMD are expected to be nearly 288 million by the year 2040.

There is a need for novel treatments for preventing progression from early to intermediate and/or from intermediate to advanced stages of AMD to prevent loss of vision.

### SUMMARY OF THE DISCLOSURE

In some embodiments, the disclosure provides for an adeno-associated viral (AAV) vector encoding a human Complement Factor I (CFI) protein or biologically active fragment thereof, wherein the vector comprises a nucleotide sequence that is at least 70% identical to the nucleotide sequence of SEQ ID NO: 1-3, 5 or 34, or codon-optimized variant and/or a fragment thereof. In some embodiments, the disclosure provides for an adeno-associated viral (AAV) vector encoding a human Complement Factor I (CFI) protein or biologically active fragment thereof, wherein the vector comprises a nucleotide sequence that is at least 80% identical to the nucleotide sequence of SEQ ID NO: 1-3, 5 or 34, or codon-optimized variant and/or a fragment thereof. In some embodiments, the nucleotide sequence is at least 90% identical to the nucleotide sequence of SEQ ID NO: 1-3, 5 or 34, or codon-optimized variant and/or a fragment thereof. In some embodiments, the nucleotide sequence is at least 95% identical to the nucleotide sequence of SEQ ID NO: 1-3, 5 or 34, or codon-optimized variant and/or a fragment thereof. In some embodiments, the nucleotide sequence is the sequence of SEQ ID NO: 1-3, 5 or 34, or codon-optimized variant and/or a fragment thereof. In some embodiments, the nucleotide sequence is at least 70%, 75%, 80%, 85%, 90%, 91%, 92%, 93%, 94%, 95%, 96%, 97%, 98%, 99% or 100% identical to the sequence of SEQ ID NO: 34. In some embodiments, the vector encodes a CFI protein or biologically active fragment thereof comprising a heavy chain and a light chain. In some embodiments, the vector encodes a CFI protein or biologically active fragment thereof comprising a FIMAC domain. In some embodiments, the vector encodes a CFI protein or biologically active fragment thereof comprising a Scavenger Receptor Cysteine Rich (SRCR) domain. In some embodiments, the vector encodes a CFI protein or biologically active fragment thereof comprising at least one LDL receptor Class A domain. In some embodiments, the vector encodes a CFI protein or biologically active fragment thereof comprising two LDL receptor Class A domains. In some embodiments, the vector encodes a CFI protein or biologically active fragment thereof comprising a serine protease domain. In some embodiments, the vector encodes a CFI protein or biologically active fragment thereof comprising a FIMAC domain, a Scavenger Receptor Cysteine Rich (SRCR) domain, and two LDL receptor Class A domains. In some embodiments, the vector encodes a CF protein or biologically active fragment thereof capable of cleaving C3b and C4b proteins. In some embodiments, the vector encodes a CFI protein or biologically active fragment thereof capable of inhibiting the assembly of C3 and C5 convertase enzymes. In some embodiments, the vector comprises a promoter that is at least 1000 nucleotides in length. In some embodiments, the vector comprises a promoter that is at least 1500 nucleotides in length. In some embodiments, the promoter comprises a nucleotide sequence that is at least 70%, 75%, 80%, 85%, 90%, 91%, 92%, 93%, 94%, 95%, 96%, 97%, 98%, 99%, or 100% identical to the nucleotide sequence of any one of SEQ ID NOs: 6, 8, 9, 11, 12, 13, 15, 17, 19, 21, 23, 25, or 27. In some embodiments, the promoter comprises a nucleotide sequence that is at least 70%, 75%, 80%, 85%, 90%, 91%, 92%, 93%, 94%, 95%, 96%, 97%, 98%, 99%, or 100% identical to the nucleotide sequence of SEQ ID NO: 19, or a fragment thereof. In some embodiments, the promoter comprises the nucleotide sequence of SEQ ID NO: 19, or a fragment thereof. In some embodiments, the vector comprises a promoter comprising a sequence that is at least 90%, 95% or 97% identical to the nucleotide sequence of SEQ ID NO: 6, or a functional fragment thereof. In some embodiments, the promoter comprises a promoter having the nucleotide sequence of SEQ ID NO: 6, or a fragment thereof. In some embodiments, the vector comprises a promoter comprising a sequence that is at least 90%, 95% or 97% identical to the nucleotide sequence of SEQ ID NO: 8, or a functional fragment thereof. In some embodiments, the promoter comprises a promoter having the nucleotide sequence of SEQ ID NO: 8, or a fragment thereof. In some embodiments, the vector comprises a promoter comprising a sequence that is at least 90%, 95% or 97% identical to the nucleotide sequence of SEQ ID NO: 9, or a functional fragment thereof. In some embodiments, the promoter comprises a promoter having the nucleotide sequence of SEQ ID NO: 9, or a fragment thereof. In some embodiments, the vector comprises a promoter comprising a sequence that is at least 90%, 95% or 97% identical to the nucleotide sequence of SEQ ID NO: 11, or a functional fragment thereof. In some embodiments, the promoter comprises a promoter having the nucleotide sequence of SEQ ID NO: 11, or a fragment thereof. In some embodiments, the vector comprises a promoter comprising a sequence that is at least 90%, 95% or 97% identical to the nucleotide sequence of SEQ ID NO: 12, or a functional fragment thereof. In some embodiments, the promoter comprises a promoter having the nucleotide sequence of SEQ ID NO: 12, or a fragment thereof. In some embodiments, the vector comprises a promoter comprising a sequence that is at least 90%, 95% or 97% identical to the nucleotide sequence of SEQ ID NO: 13, or a functional fragment thereof. In some embodiments, the promoter comprises a promoter having the nucleotide sequence of SEQ ID NO: 13, or a fragment thereof. In some embodiments, the vector comprises a promoter comprising a sequence that is at least 90%, 95% or 97% identical to the nucleotide sequence of SEQ ID NO: 15, or a functional fragment thereof. In some embodiments, the promoter comprises a promoter having the nucleotide sequence of SEQ ID NO: 15, or a fragment thereof. In some embodiments, the vector comprises a promoter comprising a sequence that is at least 90%, 95% or 97% identical to the nucleotide sequence of SEQ ID NO: 17, or a functional fragment thereof. In some embodiments, the promoter comprises a promoter having the nucleotide sequence of SEQ ID NO: 17, or a fragment thereof. In some embodiments, the vector comprises a promoter comprising a sequence that is at least 90%, 95% or 97% identical to the nucleotide sequence of SEQ ID NO: 19, or a functional fragment thereof. In some embodiments, the promoter comprises a promoter having the nucleotide sequence of SEQ ID NO: 19, or a fragment thereof. In some embodiments, the vector comprises a promoter comprising a sequence that is at least 90%, 95% or 97% identical to the nucleotide sequence of SEQ ID NO: 21, or a functional fragment thereof. In some embodiments, the promoter comprises a promoter having the nucleotide sequence of SEQ ID NO: 21, or a fragment thereof. In some embodiments, the vector comprises a promoter comprising a sequence that is at least 90%, 95% or 97% identical to the nucleotide sequence of SEQ ID NO: 23, or a functional fragment thereof. In some embodiments, the promoter comprises a promoter having the nucleotide sequence of SEQ ID NO: 23, or a fragment thereof. In some embodiments, the vector comprises a promoter comprising a sequence that is at least 90%, 95% or 97% identical to the nucleotide sequence of SEQ ID NO: 25, or a functional fragment thereof. In some embodiments, the promoter comprises a promoter having the nucleotide sequence of SEQ ID NO: 25, or a fragment thereof. In some embodiments, the vector comprises a promoter comprising a sequence that is at least 90%, 95% or 97% identical to the nucleotide sequence of SEQ ID NO: 27, or a functional fragment thereof. In some embodiments, the promoter comprises a promoter having the nucleotide sequence of SEQ ID NO: 27, or a fragment thereof. In some embodiments, the vector comprises a promoter comprising the nucleotide sequence of SEQ ID NO: 6. In some embodiments, the vector is an AAV2 vector. In some embodiments, the vector is an AAV8 vector. In some embodiments, the vector is an AAV.7m8 vector. In some embodiments, the vector comprises a CMV promoter. In some embodiments, the vector comprises a Kozak sequence. In some embodiments, the vector comprises one or more ITR sequence flanking the vector portion encoding CFI. In some embodiments, the vector comprises a polyadenylation sequence. In some embodiments, the vector comprises a selective marker. In some embodiments, the selective marker is an antibiotic-resistance gene. In some embodiments, the antibiotic-resistance gene is an ampicillin-resistance gene. In some embodiments, the antibiotic-resistance gene is a kanamycin-resistance gene.

In some embodiments, the disclosure provides for a vector, wherein the vector is an AAV2 vector, wherein the vector comprises a CFI-encoding nucleotide sequence that is at least 70%, 75%, 80%, 85%, 90%, 91%, 92%, 93%, 94%, 95%, 96%, 97%, 98%, 99% or 100% identical to the sequence of SEQ ID NO: 34; wherein the vector further comprises a promoter nucleotide sequence that is at least 70%, 75%, 80%, 85%, 90%, 91%, 92%, 93%, 94%, 95%, 96%, 97%, 98%, 99% or 100% identical to the sequence of SEQ ID NO: 19; wherein the vector encodes a CF protein comprising an A300T mutation as compared to the amino acid sequence of SEQ ID NO: 29; and wherein the CFI protein encoded by the vector is capable of cleaving C3b into iC3b. In some embodiments, the vector comprises one or more ITR sequences flanking the vector portion encoding CFI. In some embodiments, the vector comprises a polyadenylation sequence. In some embodiments, the vector comprises an SV40polyA nucleotide sequence. In some embodiments, the vector comprises a kanamycin-resistance gene. In some embodiments, the vector comprises a nucleotide sequence that is at least 70%, 75%, 80%, 85%, 90%, 91%, 92%, 93%, 94%, 95%, 96%, 97%, 98%, 99%, or 100% identical to the nucleotide sequence of SEQ ID NO: 33, or a functional fragment thereof. In some embodiments, the vector comprises the nucleotide sequence of SEQ ID NO: 33.

In some embodiments, the disclosure provides for a composition comprising any of the AAV vectors disclosed herein and a pharmaceutically acceptable carrier. In some embodiments, the composition does not comprise a protease or a polynucleotide encoding a protease. In some embodiments, the composition does not comprise a furin protease or a polynucleotide encoding a furin protease. In some embodiments, the vector in the composition is an AAV2 vector, wherein the vector comprises a CFI-encoding nucleotide sequence that is at least 70%, 75%, 80%, 85%, 90%, 91%, 92%, 93%, 94%, 95%, 96%, 97%, 98%, 99% or 100% identical to the sequence of SEQ ID NO: 34; wherein the vector further comprises a promoter nucleotide sequence that is at least 70%, 75%, 80%, 85%, 90%, 91%, 92%, 93%, 94%, 95%, 96%, 97%, 98%, 99% or 100% identical to the sequence of SEQ ID NO: 19; wherein the vector encodes a CFI protein comprising an A300T mutation as compared to the amino acid sequence of SEQ ID NO: 29; and wherein the CFI protein encoded by the vector is capable of cleaving C3b into iC3b. In some embodiments, the vector comprises one or more ITR sequences flanking the vector portion encoding CFI. In some embodiments, the vector comprises a polyadenylation sequence. In some embodiments, the vector comprises an SV40polyA nucleotide sequence. In some embodiments, the vector comprises a kanamycin-resistance gene. In some embodiments, the vector comprises a nucleotide sequence that is at least 70%, 75%, 80%, 85%, 90%.91%, 92%, 93%, 94%, 95%, 96%, 97%, 98%, 99%, or 100% identical to the nucleotide sequence of SEQ ID NO: 33, or a functional fragment thereof. In some embodiments, the vector comprises the nucleotide sequence of SEQ ID NO: 33.

In some embodiments, the disclosure provides for a method of treating a subject having a disorder associated with undesired activity of the alternative complement pathway, comprising the step of administering to the subject any of the vectors disclosed herein or any of the compositions disclosed herein. In some embodiments, the disclosure provides for a method of treating a subject having age-related macular degeneration (AMD), comprising the step of administering to the subject any of the vectors disclosed herein or any of the compositions disclosed herein. In some embodiments, the vector or composition is administered intravitreally. In some embodiments, the subject is not administered a protease or a polynucleotide encoding a protease. In some embodiments, the subject is not administered a furin protease or a polynucleotide encoding a furin protease. In some embodiments, the subject is a human. In some embodiments, the human is at least 40 years of age. In some embodiments, the human is at least 50 years of age. In some embodiments, the human is at least 65 years of age. In some embodiments, the vector or composition is administered locally. In some embodiments, the vector or composition is administered systemically. In some embodiments, the vector or composition comprises a promoter that is associated with strong expression in the liver. In some embodiments, the promoter comprises a nucleotide sequence that is at least 90%, 95% or 100% identical to the nucleotide sequence of any one of SEQ ID NOs: 13, 15 or 27. In some embodiments, the vector or composition comprises a promoter that is associated with strong expression in the eye. In some embodiments, the promoter comprises a nucleotide sequence that is at least 90%, 95%, or 100% identical to the nucleotide sequence of any one of SEQ ID NOs: 21 or 25. In some embodiments, the subject has a loss-of-function mutation in the subject's CFI gene. In some embodiments, the subject has one or more CF mutations selected from the group consisting of: G119R, L131R, V152M, G162D, R187Y, R187T, T203I, A240G, A258T, G287R, A300T, R317W, R339Q, V412M, and P553S. In some embodiments, the subject has a loss-of-function mutation in the subject's CFH gene. In some embodiments, the subject has one or more CFH mutations selected from the group consisting of: R2T, L3V, R53C, R53H, S58A, G69E, D90G, R175Q, S193L, I216T, I221V, R303W, H402Y, Q408X, P503A, G650V, R1078S, and R1210C. In some embodiments, the subject has atypical hemolytic uremic syndrome (aHUS). In some embodiments, the subject is suffering from a renal disease or complication. In some embodiments, the vector for use in any of the methods disclosed herein is an AAV2 vector, wherein the vector comprises a CFI-encoding nucleotide sequence that is at least 70%, 75%, 80%, 85%, 90%, 91%, 92%, 93%, 94%, 95%, 96%, 97%, 98%, 99% or 100% identical to the sequence of SEQ ID NO: 34; wherein the vector further comprises a promoter nucleotide sequence that is at least 70%, 75%, 80%, 85%, 90%, 91%, 92%, 93%, 94%, 95%, 96%, 97%, 98%, 99% or 100% identical to the sequence of SEQ ID NO: 19; wherein the vector encodes a CF protein comprising an A300T mutation as compared to the amino acid sequence of SEQ ID NO: 29; and wherein the CFI protein encoded by the vector is capable of cleaving C3b into iC3b. In some embodiments, the vector comprises one or more ITR sequences flanking the vector portion encoding CFI. In some embodiments, the vector comprises a polyadenylation sequence. In some embodiments, the vector comprises an SV40polyA nucleotide sequence. In some embodiments, the vector comprises a kanamycin-resistance gene. In some embodiments, the vector comprises a nucleotide sequence that is at least 70%, 75%, 80%, 85%, 90%, 91%, 92%, 93%, 94%, 95%, 96%, 97%, 98%, 99%, or 100% identical to the nucleotide sequence of SEQ ID NO: 33, or a functional fragment thereof. In some embodiments, the vector comprises the nucleotide sequence of SEQ ID NO: 33. In some embodiments, the subject to be treated with the method has a P553S CFI mutation. In some embodiments, the subject has a K441R CFI mutation. In some embodiments, the subject has an R339Q CFI mutation. In some embodiments, the subject has an R339Ter CF mutation. In some embodiments, the subject has an R317Q CFI mutation. In some embodiments, the subject has an R317W CFI mutation. In some embodiments, the subject has an A300T CFI mutation. In some embodiments, the subject has a G287R CFI mutation. In some embodiments, the subject has a G261D CFI mutation. In some embodiments, the subject has an A258T CFI mutation. In some embodiments, the subject has an A240G CFI mutation. In some embodiments, the subject has a T203I CFI mutation. In some embodiments, the subject has an R187Q CFI mutation. In some embodiments, the subject has an R187Ter CFI mutation. In some embodiments, the subject has a G162D CFI mutation. In some embodiments, the subject has a V152M CFI mutation. In some embodiments, the subject has a G119R CFI mutation. In some embodiments, the subject is homozygous for the CFI mutation. In some embodiments, the subject is heterozygous for the CFI mutation. In some embodiments, the subject expresses a mutant CFI protein having reduced CFI activity as compared to a wildtype CFI protein (e.g., a CFI protein having the amino acid sequence of SEQ ID NO: 29). In some embodiments, the CF activity is the ability to cleave C3b to iC3b. In some embodiments, if a CFI protein having the CFI mutation were tested in a functional assay, the mutant CFI protein would display reduced CFI activity as compared to a wildtype CFI protein (e.g., a CFI protein having the amino acid sequence of SEQ ID NO: 29). In some embodiments, the functional assay tests the ability of CFI to cleave C3b to iC3b. In some embodiments, the vector or composition is administered to the retina at a dose in the range of 1×10¹⁰ vg/eye to 1×10¹³ vg/eye. In some embodiments, the vector or composition is administered to the retina at a dose of about 1.4×10¹² vg/eye. In some embodiments, the CFI is processed to an active CFI. In some embodiments, the subject is a subject in whom it has been determined has one or more CFI mutations. In some embodiments, the subject is a subject in whom it has been determined has one or more CFI mutations selected from the group consisting of: G119R, L131R, V152M, G162D, R187Y, R187T, T203I, A240G, A258T, G287R, A300T, R317W, R339Q, V412M, and P553S. In some embodiments, the subject is a subject in whom it has been determined has one or more CF mutations selected from the group consisting of: P553S, K441R, R339Q, R339Ter, R317Q, R317W, A300T, G287R, G261D, A258T, A240G, T203I, R187Q, R187Ter, G162D, V152M, or G119R. In some embodiments, the subject is a subject in whom it has been determined has a P553S CFI mutation. In some embodiments, the subject is a subject in whom it has been determined has a K441R CFI mutation. In some embodiments, the subject is a subject in whom it has been determined has an R339Q CFI mutation. In some embodiments, the subject is a subject in whom it has been determined has an R339Ter CFI mutation. In some embodiments, the subject is a subject in whom it has been determined has an R317Q CFI mutation. In some embodiments, the subject is a subject in whom it has been determined has an R317W CFI mutation. In some embodiments, the subject is a subject in whom it has been determined has an A300T CFI mutation. In some embodiments, the subject is a subject in whom it has been determined has a G287R CFI mutation. In some embodiments, the subject is a subject in whom it has been determined has a G261D CFI mutation. In some embodiments, the subject is a subject in whom it has been determined has an A258T CFI mutation. In some embodiments, the subject is a subject in whom it has been determined has an A240G CFI mutation. In some embodiments, the subject is a subject in whom it has been determined has a T203I CFI mutation. In some embodiments, the subject is a subject in whom it has been determined has an R187Q CFI mutation. In some embodiments, the subject is a subject in whom it has been determined has an R187Ter CFI mutation. In some embodiments, the subject is a subject in whom it has been determined has a G162D CFI mutation. In some embodiments, the subject is a subject in whom it has been determined has a V152M CFI mutation. In some embodiments, the subject is a subject in whom it has been determined has a G119R CFI mutation. In some embodiments, the subject is a subject in whom it has been determined is homozygous for at least one of the one or more CFI mutations. In some embodiments, the subject is a subject in whom it has been determined is heterozygous for at least one of the one or more CFI mutations.

In some embodiments, any of the vectors disclosed herein is capable of inducing at least 20%, 50%, 100%, 150%, 200%, 250%, 300%, 400%, 500%, 700%, 900%, 1000%, 1100%, 1500%, or 2000% expression of CFI in a target cell (e.g., an RPE or liver cell) as compared to the endogenous expression of CFI in the target cell. In some embodiments, expression of any of the vectors disclosed herein in a target cell (e.g., an RPE or liver cell) results in at least 20%, 50%, 100%, 150%, 200%, 250%, 300%, 400%, 500%, 700%, 900%, 1000%, 1100%, 1500%, or 2000% levels of CFI activity in the target cell as compared to endogenous levels of CFI activity in the target cell. In some embodiments, any of the vectors or compositions disclosed herein induces CFI expression in a target cell of the eye. In some embodiments, the vector or composition induces CFI expression in a target cell of the retina or macula. In some embodiments, the target cell of the retina is selected from the group of layers consisting of: inner limiting membrane, nerve fiber, ganglion cell layer (GCL), inner plexiform layer, inner nuclear layer, outer plexiform layer, outer nuclear layer, external limiting membrane, rods and cones, and retinal pigment epithelium (RPE). In some embodiments, the target cell is in the choroid plexus. In some embodiments, the target cell is in the macula. In some embodiments, the vector or composition induces CFI expression in a cell of the GCL and/or RPE. In some embodiments, the CFI is processed to an active CFI. In some embodiments, the vector or composition is administered to the retina at a dose in the range of 1×10¹⁰ vg/eye to 1×10¹³ vg/eye. In some embodiments, the vector or composition is administered to the retina at a dose of about 1.4×10¹² vg/eye. In some embodiments, the CFI is processed to an active CFI.

### BRIEF DESCRIPTION OF THE DRAWINGS

FIG. 1 shows a vector map of a full vector genome construct for expression of CFI. “ITR” corresponds to inverted terminal repeats; “CBA” corresponds to the chicken β actin promoter: “CFI” corresponds to the gene encoding Complement Factor 1: “polyA” corresponds to the polyadenylation sequence; “AmpicillinR” corresponds to the ampicillin resistance cassette. The nucleotide sequence corresponding to the vector illustrated in FIG. 1 is SEQ ID NO: 7.

FIG. 2 shows a vector map of a full vector genome construct for expression of CFI. “ITR” corresponds to inverted terminal repeats; “AAT1” corresponds to the alpha1 antitrypsin promoter; “CFI” corresponds to the gene encoding Complement Factor I: “polyA” corresponds to the polyadenylation sequence; “AmpR” corresponds to the ampicillin resistance cassette. The nucleotide sequence corresponding to the vector illustrated in FIG. 2 is SEQ ID NO: 14.

FIG. 3 shows a vector map of a full vector genome construct for expression of CFI. “ITR” corresponds to inverted terminal repeats; “ALB” corresponds to a synthetic promoter based on the human albumin promoter; “CFI” corresponds to the gene encoding Complement Factor I; “polyA” corresponds to the polyadenylation sequence; “AmpR” corresponds to the ampicillin resistance cassette. The nucleotide sequence corresponding to the vector illustrated in FIG. 3 is SEQ ID NO: 16.

FIG. 4 shows a vector map of a full vector genome construct for expression of CFI. “ITR” corresponds to inverted terminal repeats; “CAG” corresponds to a synthetic promoter that includes the cytomegalovirus (CMV) early enhancer element, the promoter/first exon/first intron of chicken beta-actin gene, and the splice acceptor of the rabbit beta-globin gene; “CFI” corresponds to the gene encoding Complement Factor I; “polyA” corresponds to the polyadenylation sequence; “AmpR” corresponds to the ampicillin resistance cassette. The nucleotide sequence corresponding to the vector illustrated in FIG. 4 is SEQ ID NO: 18.

FIG. 5 shows a vector map of a full vector genome construct for expression of CFI. “ITR” corresponds to inverted terminal repeats, “CBA” corresponds to the chicken β actin promoter; “CFI” corresponds to the gene encoding Complement Factor I; “polyA” corresponds to the polyadenylation sequence; “AmpR” corresponds to the ampicillin resistance cassette. The nucleotide sequence corresponding to the vector illustrated in FIG. 5 is SEQ ID NO: 20.

FIG. 6 shows a vector map of a full vector genome construct for expression of CFI. “ITR” corresponds to inverted terminal repeats; “CRALBP promoter” corresponds to the cellular retinaldehyde-binding protein promoter; “CFI” corresponds to the gene encoding Complement Factor I; “polyA” corresponds to the polyadenylation sequence; “AmpicillinR” corresponds to the ampicillin resistance cassette. The nucleotide sequence corresponding to the vector illustrated in FIG. 6 is SEQ ID NO: 22.

FIG. 7 shows a vector map of a full vector genome construct for expression of CFI. “ITR” corresponds to inverted terminal repeats; “EF1a promoter” corresponds to the elongation factor-1 alpha promoter; “CFI” corresponds to the gene encoding Complement Factor I; “polyA” corresponds to the polyadenylation sequence; “AmpicillinR” corresponds to the ampicillin resistance cassette. The nucleotide sequence corresponding to the vector illustrated in FIG. 7 is SEQ ID NO: 24.

FIG. 8 shows a vector map of a full vector genome construct for expression of CFI. “ITR” corresponds to inverted terminal repeats; “RPE65 promoter” corresponds to the retinal pigment epithelial 65 promoter; “CFI” corresponds to the gene encoding Complement Factor I; “polyA” corresponds to the polyadenylation sequence; “AmpicillinR” corresponds to the ampicillin resistance cassette. The nucleotide sequence corresponding to the vector illustrated in FIG. 7 is SEQ ID NO: 26.

FIG. 9 shows a vector map of a full vector genome construct for expression of CFI. “ITR” corresponds to inverted terminal repeats; “PCK1 promoter” corresponds to the Phosphoenolpyruvate carboxykinase 1 promoter; “CFI” corresponds to the gene encoding Complement Factor I; “polyA” corresponds to the polyadenylation sequence; “AmpicillinR” corresponds to the ampicillin resistance cassette. The nucleotide sequence corresponding to the vector illustrated in FIG. 8 is SEQ ID NO: 28.

FIG. 10 shows an image of a gel from a Western Blot analysis. Lanes 1 and 10 correspond to ladder markers, lane 2 corresponds to 50 ng of recombinant CF protein, lane 3 corresponds to vitreous humor from left eye of vehicle treatment animal, lane 4 corresponds to vitreous humor from left eye of vehicle treatment animal with 100 ng of recombinant CFI protein added directly prior to Western blotting, lane 5 is a blank lane, lane 6 corresponds to vitreous humor from right eye of an animal treated with AAV2-CBA-CFI virus, lane 7 corresponds to vitreous humor from left eye of an animal treated with AAV2-CBA-CFI virus, lane 8 corresponds to vitreous humor from right eye of an additional animal treated with AAV2-CBA-CFI virus, and lane 9 corresponds to vitreous humor from a human donor.

FIG. 11 shows an image of a gel from a Western Blot analysis. Lanes 1 and 10 correspond to ladder markers; lane 2 corresponds to 25 ng of recombinant CF protein, lane 3 corresponds to RPE/choroid from left eye of vehicle treatment animal, lane 4 corresponds to RPE/choroid from left eye of vehicle treatment animal with 25 ng of recombinant CFI protein added directly prior to Western blotting, lane 5 is a blank lane, lane 6 corresponds to RPE/choroid from left eye of an animal treated with AAV2-CBA-CFI virus, lane 7 corresponds to RPE/choroid from right eye of an animal treated with AAV2-CBA-CFI virus, lane 8 corresponds to RPE/choroid from right eye of an additional animal treated with AAV2-CBA-CFI virus, and lane 9 corresponds to RPE/choroid from a human donor.

FIG. 12 is a graph showing the results of a co-factor assay using treated and untreated animals. The slope for the vehicle control sample is −0.28±0.02, the slope for the treated OD (right eye) and OS (left eye) samples is −0.47±0.02, and the slope of the CFI control sample is −0.75±0.02.

FIG. 13A shows the quantification of CFI protein using the stand curve generated using a human specific F1 Microvue kit (A041, Quidel Corporation) with the kit standards by linear regression using Graphpad Prism software. FIG. 13B is a table listing the concentration (ng/ml) of test article (either vehicle control or AAV2-CFI) administered intravitreally to cynomolgus monkeys. FIG. 13C shows the levels of CFI protein in vitreous humor samples obtained from left (L) or right (R) eye samples from each of the treated animals as detected using the CF ELISA assay. FIG. 13D shows the average amount of CF protein in vitreous humor samples from each treated animal as detected using the CFI ELISA assay. FIG. 13E summarizes the level of CFI protein across the entire experiment, with each dot representing the CFI level in the vitreous humor of one eye. The green line represents half of the level of CF protein in the vitreous humor of the normal human population.

FIG. 14A shows the quantification of CFI protein using the stand curve generated using a human specific FI Microvue kit (A041, Quidel Corporation) with the kit standards by linear regression using Graphpad Prism software. FIG. 14B is a table listing the concentration (ng/ml) of test article (either vehicle control or AAV2-CFI) administered intravitreally to cynomolgus monkeys. FIG. 14C shows the levels of CFI protein in aqueous humor samples obtained from left (L) or right (R) eye samples from each of the treated animals as detected using the CFI ELISA assay. FIG. 14D shows the average amount of CFI protein in aqueous humor samples from each treated animal as detected using the CFI ELISA assay. FIG. 14E summarizes the level of CF protein across the entire experiment, with each dot representing the CFI level in the aqueous humor of one eye. The green line represents half of the level of CFI protein in the aqueous humor of the normal human population.

FIG. 15 is a graph showing the correlation between CFI levels detected at different concentrations in aqueous humor and vitreous humor samples obtained from treated animals.

FIG. 16A is a graph showing the percent relative fluorescence units (RFU) normalized to 100% for levels of active CFI detected in vitreous humor samples obtained from cynomolgus monkeys that were intravitreally administered different doses of CFI-AAV vector. FIG. 16B is a graph showing the maximum reaction rates (Vmax) for each sample as calculated using Graphpad Prism software based on the analysis of the nonlinear regression of the kinetic activity data from 500s to 1800s. The slopes were graphed as inverse RFU/second. “Neat cyno VH” corresponds to undiluted cynomolgus vitreous humor.

FIGS. 17A and 17B are graphs showing the percent relative fluorescence units (RFUs) normalized to 100% for levels of active CFI detected in vitreous humor samples obtained from cynomolgus monkeys that were intravitreally administered different doses of CFI-AAV vector. FIG. 17A is based on data obtained from testing vitreous humor samples from right (R) or (L) eyes of six different animals tested. FIG. 17B is based on data obtained from testing vitreous humor samples from right (R) or (L) eyes of two different animals tested. Amounts of vector administered to each animal eye is indicated in FIG. 13B. The kinetic plots were analyzed by assessment of the slopes. The reaction rates, i.e., the slopes of observed reduction in fluorescence at 472 nm (corresponding to C3b cleavage), were calculated for each sample, carried out in triplicate. The maximum reaction rates (Vmax) for each sample were calculated by Graphpad Prism software based on the analysis of the nonlinear regression of the kinetic activity data from 500s to 1800s. The slopes were graphed as inverse RFU/second and are shown in FIGS. 16B, 17C and 17D. Activity levels of different concentrations of CFI were tested in FIG. 16A were calculated for 16B; activity levels of CFI from the samples tested in FIG. 17A were calculated for 17C; and in FIG. 17D the relationship between the levels of CFI protein detected in the vitreous humor after dosing with AAV-CFI (as shown in FIGS. 13B-13E) and the Vmax of CFI activity in vitreous humor (FIG. 17C).

FIG. 18A shows the expression of GFP protein following administration of our AAV2-GFP construct in the eye of NHPs treated with the AAV2 by intravitreal administration. FIG. 18B shows the level of expression of CFI protein as determined by ELISA (as described above) in various levels of the retina from animals treated with AAV2-CFI. The retina was dissected into layers by standard methods, the tissue was homogenized and CFI protein detected by ELISA as described above.

FIG. 19 shows a vector map of a full vector genome construct for expression of CFI. “ITR” corresponds to inverted terminal repeats; “CBA” corresponds to the chicken p actin promoter: “CFI” corresponds to the gene encoding Complement Factor I (including alanine at the position corresponding to position 300 of SEQ ID NO: 35); “polyA” corresponds to the polyadenylation sequence; “KanR” corresponds to the kanamycin resistance cassette. “Ori” corresponds to the origin of replication. Various restriction enzyme sites are indicated in the vector map. The nucleotide sequence corresponding to the vector illustrated in FIG. 19 is SEQ ID NO: 33.

FIG. 20 shows gel images from a series of Western Blots. “Std” corresponds to the molecular weight standard. The arrow points to the mature form of CFI. Lane 3 contains conditioned medium from negative control cells that did not overexpress CFI constructs.

FIG. 21 shows a series of graphs from fluorescence cofactor assays. In each assay, increasing concentrations of wildtype CFI or G119R CFI protein were mixed with a different cofactor (CFH, MCP or CR1) and with ANS-labeled C3b, and relative fluorescent units (RFUs) were then measured over time and plotted against the concentration of CFI protein in ug/ml.

FIG. 22 shows a series of graphs from fluorescence cofactor assays. In each assay, increasing concentrations of wildtype CFI or A240G CFI protein were mixed with a different cofactor (CFH, MCP or CR1) and with ANS-labeled C3b, and relative fluorescent units (RFUs) were then measured over time and plotted against the concentration of CFI protein in ug/ml.

FIG. 23 shows a series of graphs from fluorescence cofactor assays. In each assay, increasing concentrations of wildtype CFI or P553S CFI protein were mixed with a different cofactor (CFH, MCP or CR1) and with ANS-labeled C3b, and relative fluorescent units (RFUs) were then measured over time and plotted against the concentration of CFI protein in ug/ml.

FIG. 24 shows a series of graphs from fluorescence cofactor assays. In each assay, increasing concentrations of wildtype CFI or A300T CFI protein were mixed with a different cofactor (CFH, MCP or CR1) and with ANS-labeled C3b, and relative fluorescent units (RFUs) were then measured over time and plotted against the concentration of CFI protein in ug/ml.

FIG. 25 shows a series of graphs from fluorescence cofactor assays. In each assay, increasing concentrations of CFH cofactor protein were mixed with wildtype CFI or a CFI mutant (G119R, A240G, A300T or P553S) and with ANS-labeled C3b, and relative fluorescent units (RFUs) were then measured over time and plotted against the concentration of CFI protein in ug/ml.

### DETAILED DESCRIPTION OF THE DISCLOSURE

The disclosure provides compositions and methods for treating, preventing, or inhibiting diseases of the eye. In one aspect, the disclosure provides recombinant adeno-associated virus (rAAV) vectors comprising a complement system gene (such as, but not limited to genes encoding complement factor I (CFI). In another aspect, the disclosure provides methods of treating, preventing, or inhibiting diseases of the eye by intraocularly (e.g., intravitreally) administering an effective amount of an rAAV vector of the disclosure to deliver and drive the expression of a complement factor gene.

A wide variety of diseases of the eye may be treated or prevented using the viral vectors and methods provided herein. Diseases of the eye that may be treated or prevented using the vectors and methods of the disclosure include but are not limited to, glaucoma, macular degeneration (e.g., age-related macular degeneration), diabetic retinopathies, inherited retinal degeneration such as retinitis pigmentosa, retinal detachment or injury and retinopathies (such as retinopathies that are inherited, induced by surgery, trauma, an underlying aetiology such as severe anemia, SLE, hypertension, blood dyscrasias, systemic infections, or underlying carotid disease, a toxic compound or agent, or photically).

General Techniques

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press: Oligonucleotide Synthesis (M. J. Gait, ed., 1984); Methods in Molecular Biology, Humana Press: Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press: Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987): PCR: The Polymerase Chain Reaction, (Mullis et al., eds., 1994): Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons. NY (2003); Short Protocols in Molecular Biology (Wiley and Sons, 1999).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well known and commonly used in the art. Standard techniques are used for chemical syntheses, and chemical analyses.

Throughout this specification and embodiments, the word “comprise,” or variations such as “comprises” or “comprising.” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10: that is, all subranges beginning with a minimum value of 1 or more, e.g., 1 to 6.1, and ending with a maximum value of 10 or less, e.g., 5.5 to 10. Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the disclosure.

Exemplary methods and materials are described herein, although methods and materials similar or equivalent to those described herein can also be used in the practice or testing of the present disclosure. The materials, methods, and examples are illustrative only and not intended to be limiting.

### Definitions

The following terms, unless otherwise indicated, shall be understood to have the following meanings:

As used herein, “residue” refers to a position in a protein and its associated amino acid identity.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” or “percent (%) identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, a “host cell” includes an individual cell or cell culture that can be or has been a recipient for vector(s) for incorporation of polynuclcotide inserts. The term host cell may refer to the packaging cell line in which the rAAV is produced from the plasmid. In the alternative, the term “host cell” may refer to the target cell in which expression of the transgene is desired.

As used herein, a “vector,” refers to a recombinant plasmid or virus that comprises a nucleic acid to be delivered into a host cell, either in vitro or in vivo. A “recombinant viral vector” refers to a recombinant polynucleotide vector comprising one or more heterologous sequences (i.e. a nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs.

A “recombinant AAV vector (rAAV vector)” refers to a polynucleotide vector based on an adeno-associated virus comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral particle, e.g., an AAV particle. An rAAV vector can be packaged into an AAV virus capsid to generate a “recombinant adeno-associated viral particle (rAAV particle)”.

An “rAAV virus” or “rAAV viral particle” refers to a viral particle composed of at least one AAV capsid protein and an encapsidated rAAV vector genome.

The term “transgene” refers to a polynuclcotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as miRNA, siRNA, or shRNA.

The term “vector genome (vg)” as used herein may refer to one or more polynucleotides comprising a set of the polynucleotide sequences of a vector, e.g., a viral vector. A vector genome may be encapsidated in a viral particle. Depending on the particular viral vector, a vector genome may comprise single-stranded DNA, double-stranded DNA, or single-stranded RNA, or double-stranded RNA. A vector genome may include endogenous sequences associated with a particular viral vector and/or any heterologous sequences inserted into a particular viral vector through recombinant techniques. For example, a recombinant AAV vector genome may include at least one ITR sequence flanking a promoter, a stuffer, a sequence of interest (e.g., an RNAi), and a polyadenylation sequence. A complete vector genome may include a complete set of the polynucleotide sequences of a vector. In some embodiments, the nucleic acid titer of a viral vector may be measured in terms of vg/mL. Methods suitable for measuring this titer are known in the art (e.g., quantitative PCR).

An “inverted terminal repeat” or “ITR” sequence is a term well understood in the art and refers to relatively short sequences found at the termini of viral genomes which are in opposite orientation.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C and D regions), allowing intrastrand base-pairing to occur within this portion of the ITR.

A “helper virus” for AAV refers to a virus that allows AAV (which is a defective parvovirus) to be replicated and packaged by a host cell. A number of such helper viruses are known in the art.

As used herein, “expression control sequence” means a nucleic acid sequence that directs transcription of a nucleic acid. An expression control sequence can be a promoter, such as a constitutive promoter, or an enhancer. The expression control sequence is operably linked to the nucleic acid sequence to be transcribed.

As used herein, “isolated molecule” (where the molecule is, for example, a polypeptide, a polynucleotide, or fragment thereof) is a molecule that by virtue of its origin or source of derivation (1) is not associated with one or more naturally associated components that accompany it in its native state. (2) is substantially free of one or more other molecules from the same species (3) is expressed by a cell from a different species, or (4) does not occur in nature.

As used herein, “purify,” and grammatical variations thereof, refers to the removal, whether completely or partially, of at least one impurity from a mixture containing the polypeptide and one or more impurities, which thereby improves the level of purity of the polypeptide in the composition (i.e., by decreasing the amount (ppm) of impurity(ies) in the composition).

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The terms “patient”, “subject”, or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In some embodiments, the subject is a human that is at least 40, 45, 50, 55, 60, 65, 70, 75, 80, 85, 90 or 95 years of age.

In one embodiment, the subject has, or is at risk of developing a disease of the eye. A disease of the eye, includes, without limitation, AMD, retinitis pigmentosa, rod-conc dystrophy, Leber's congenital amaurosis, Usher's syndrome, Bardet-Biedl Syndrome, Best disease, retinoschisis, Stargardt disease (autosomal dominant or autosomal recessive), untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, glaucoma, or retinal vein occlusion. In some embodiments, the subject has drusen deposits and/or geographic atrophy. In another embodiment, the subject has, or is at risk of developing glaucoma, Leber's hereditary optic neuropathy, lysosomal storage disorder, or peroxisomal disorder. In another embodiment, the subject is in need of optogenetic therapy. In another embodiment, the subject has shown clinical signs of a disease of the eye.

In some embodiments, the subject has, or is at risk of developing a renal disease or complication. In some embodiments, the renal disease or complication is associated with AMD or aHUS.

In some embodiments, the subject has, or is at risk of developing AMD or aHUS.

Clinical signs of a disease of the eye include, but are not limited to, decreased peripheral vision, decreased central (reading) vision, decreased night vision, loss of color perception, reduction in visual acuity, decreased photoreceptor function, and pigmentary changes. In one embodiment, the subject shows degeneration of the outer nuclear layer (ONL). In another embodiment, the subject has been diagnosed with a disease of the eye. In yet another embodiment, the subject has not yet shown clinical signs of a disease of the eye.

As used herein, the terms “prevent”, “preventing” and “prevention” refer to the prevention of the recurrence or onset of, or a reduction in one or more symptoms of a disease or condition (e.g., a disease of the eye) in a subject as result of the administration of a therapy (e.g., a prophylactic or therapeutic agent). For example, in the context of the administration of a therapy to a subject for an infection, “prevent”, “preventing” and “prevention” refer to the inhibition or a reduction in the development or onset of a disease or condition (e.g., a disease of the eye), or the prevention of the recurrence, onset, or development of one or more symptoms of a disease or condition (e.g., a disease of the eye), in a subject resulting from the administration of a therapy (e.g., a prophylactic or therapeutic agent), or the administration of a combination of therapies (e.g., a combination of prophylactic or therapeutic agents).

“Treating” a condition or patient refers to taking steps to obtain beneficial or desired results, including clinical results. With respect to a disease or condition (e.g., a disease of the eye), treatment refers to the reduction or amelioration of the progression, severity, and/or duration of an infection (e.g., a disease of the eye or symptoms associated therewith), or the amelioration of one or more symptoms resulting from the administration of one or more therapies (including, but not limited to, the administration of one or more prophylactic or therapeutic agents).

“Administering” or “administration of” a substance, a compound or an agent to a subject can be carried out using one of a variety of methods known to those skilled in the art. For example, a compound or an agent can be administered intravitreally or subretinally. In particular embodiments, the compound or agent is administered intravitreally. In some embodiments, administration may be local. In other embodiments, administration may be systemic. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

As used herein, the term “ocular cells” refers to any cell in, or associated with the function of, the eye. The term may refer to any one or more of photoreceptor cells, including rod, cone and photosensitive ganglion cells, retinal pigment epithelium (RPE) cells, glial cells, Muller cells, bipolar cells, horizontal cells, amacrine cells. In one embodiment, the ocular cells are bipolar cells. In another embodiment, the ocular cells are horizontal cells. In another embodiment, the ocular cells are ganglion cells. In particular embodiments, the cells are RPE cells.

Each embodiment described herein may be used individually or in combination with any other embodiment described herein.

Construction of rAAV Vectors

The disclosure provides recombinant AAV (rAAV) vectors comprising a complement system gene (e.g. CF) or a fragment thereof, under the control of a suitable promoter to direct the expression of the complement system gene, splice variant, or fragment thereof in the eye. The disclosure further provides a therapeutic composition comprising an rAAV vector comprising a complement system gene, a splice variant, or a fragment thereof (e.g. CF1) under the control of a suitable promoter. A variety of rAAV vectors may be used to deliver the desired complement system gene to the eye and to direct its expression. More than 30 naturally occurring serotypes of AAV from humans and non-human primates are known. Many natural variants of the AAV capsid exist, and an rAAV vector of the disclosure may be designed based on an AAV with properties specifically suited for ocular cells. In certain embodiments, the complement system gene is a splice variant.

In general, an rAAV vector is comprised of, in order, a 5′ adeno-associated virus inverted terminal repeat, a transgene or gene of interest encoding a complement system polypeptide (e.g. CFI) or a biologically active fragment thereof operably linked to a sequence which regulates its expression in a target cell, and a 3′ adeno-associated virus inverted terminal repeat. In addition, the rAAV vector may preferably have a polyadenylation sequence. Generally, rAAV vectors should have one copy of the AAV ITR at each end of the transgene or gene of interest, in order to allow replication, packaging, and efficient integration into cell chromosomes. Within preferred embodiments of the disclosure, the transgene sequence encoding a complement system polypeptide (e.g. CFI) or a biologically active fragment thereof will be of about 2 to 5 kb in length (or alternatively, the transgene may additionally contain a “stuffer” or “filler” sequence to bring the total size of the nucleic acid sequence between the two ITRs to between 2 and 5 kb). Alternatively, the transgene encoding a complement system polypeptide (e.g. CFI) or a biologically active fragment thereof may be composed of the same heterologous sequence several times (e.g., two nucleic acid molecules of a complement system gene separated by a ribosomal readthrough stop codon, or alternatively, by an Internal Ribosome Entry Site or “IRES”), or several different heterologous sequences (e.g., different complement system members such as CFI, separated by a ribosomal readthrough stop codon or an IRES).

Recombinant AAV vectors of the present disclosure may be generated from a variety of adeno-associated viruses. For example, ITRs from any AAV serotype are expected to have similar structures and functions with regard to replication, integration, excision and transcriptional mechanisms. Examples of AAV serotypes include AAV1, AAV2, AAV3, AAV4, AAV5, AAV6, AAV7, AAV8, AAV9, AAV10, AAV11 and AAV12. In some embodiments, the rAAV vector is generated from serotype AAV1, AAV2, AAV4, AAV5, or AAV8. These serotypes are known to target photoreceptor cells or the retinal pigment epithelium. In particular embodiments, the rAAV vector is generated from serotype AAV2. In certain embodiments, the AAV serotypes include AAVrh8, AAVrh8R or AAVrh10. It will also be understood that the rAAV vectors may be chimeras of two or more serotypes selected from serotypes AAV1 through AAV12. The tropism of the vector may be altered by packaging the recombinant genome of one serotype into capsids derived from another AAV serotype. In some embodiments, the ITRs of the rAAV virus may be based on the ITRs of any one of AAV1-12 and may be combined with an AAV capsid selected from any one of AAV1-12, AAV-DJ, AAV-DJ8, AAV-DJ9 or other modified serotypes. In certain embodiments, any AAV capsid serotype may be used with the vectors of the disclosure. Examples of AAV serotypes include AAV1, AAV2, AAV3, AAV4, AAV5, AAV6, AAV7, AAV8, AAV9, AAV10, AAV11, AAV12, AAV-DJ, AAV-DJ8, AAV-DJ9, AAVrh8, AAVrh8R or AAVrh10. In certain embodiments, the AAV capsid serotype is AAV2. In some embodiments, the AAV capsid serotype is AAV.7m8.

In some embodiments, the AAV capsid serotype is not AAV3. In some embodiments, the vector does not comprise any AAV3 components.

Desirable AAV fragments for assembly into vectors may include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments maybe used, alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the disclosure. In some embodiments, the AAV is AAV2/5. In another embodiment, the AAV is AAV2/8. When pseudotyping an AAV vector, the sequences encoding each of the essential rep proteins may be supplied by different AAV sources (e.g., AAV1, AAV2, AAV3, AAV4, AAV5, AAV6, AAV7, AAV8). For example, the rep78/68 sequences may be from AAV2, whereas the rep52/40 sequences may be from AAV8.

In one embodiment, the vectors of the disclosure contain, at a minimum, sequences encoding a selected AAV serotype capsid, e.g., an AAV2 capsid or a fragment thereof. In another embodiment, the vectors of the disclosure contain, at a minimum, sequences encoding a selected AAV serotype rep protein, e.g., AAV2 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV2 origin. In certain embodiments, the vectors may comprise rep sequences from an AAV serotype which differs from that which is providing the cap sequences. In some embodiments, the rep and cap sequences are expressed from separate sources (e.g., separate vectors, or a host cell and a vector). In some embodiments, these rep sequences are fused in frame to cap sequences of a different AAV serotype to form a chimeric AAV vector, such as AAV2/8 described in U.S. Pat. No. 7,282,199, which is incorporated by reference herein. Examples of AAV serotypes include AAV1, AAV2, AAV3, AAV4, AAV5, AAV6, AAV7, AAV.7m8, AAV8, AAV9, AAV10, AAV11, AAV12, AAV-DJ, AAV-DJ8, AAV-DJ9, AAVrh8, AAVrh8R or AAVrh10. In some embodiments, the cap is derived from AAV2.

In some embodiments, any of the vectors disclosed herein includes a spacer, i.e., a DNA sequence interposed between the promoter and the rep gene ATG start site. In some embodiments, the spacer may be a random sequence of nucleotides, or alternatively, it may encode a gene product, such as a marker gene. In some embodiments, the spacer may contain genes which typically incorporate start/stop and polyA sites. In some embodiments, the spacer may be a non-coding DNA sequence from a prokaryote or eukaryote, a repetitive non-coding sequence, a coding sequence without transcriptional controls or a coding sequence with transcriptional controls. In some embodiments, the spacer is a phage ladder sequences or a yeast ladder sequence. In some embodiments, the spacer is of a size sufficient to reduce expression of the rep78 and rep68 gene products, leaving the rep52, rep40 and cap gene products expressed at normal levels. In some embodiments, the length of the spacer may therefore range from about 10 bp to about 10.0 kbp, preferably in the range of about 100 bp to about 8.0 kbp. In some embodiments, the spacer is less than 2 kbp in length.

In certain embodiments, the capsid is modified to improve therapy. The capsid may be modified using conventional molecular biology techniques. In certain embodiments, the capsid is modified for minimized immunogenicity, better stability and particle lifetime, efficient degradation, and/or accurate delivery of the transgene encoding the complement system polypeptide (e.g. CFI) or biologically active fragment thereof to the nucleus. In some embodiments, the modification or mutation is an amino acid deletion, insertion, substitution, or any combination thereof in a capsid protein. A modified polypeptide may comprise 1, 2, 3, 4, 5, up to 10, or more amino acid substitutions and/or deletions and/or insertions. A “deletion” may comprise the deletion of individual amino acids, deletion of small groups of amino acids such as 2, 3, 4 or 5 amino acids, or deletion of larger amino acid regions, such as the deletion of specific amino acid domains or other features. An “insertion” may comprise the insertion of individual amino acids, insertion of small groups of amino acids such as 2, 3, 4 or 5 amino acids, or insertion of larger amino acid regions, such as the insertion of specific amino acid domains or other features. A “substitution” comprises replacing a wild type amino acid with another (e.g., a non-wild type amino acid). In some embodiments, the another (e.g., non-wild type) or inserted amino acid is Ala (A), His (H), Lys (K), Phe (F), Met (M), Thr (T), Gln (Q), Asp (D), or Glu (E). In some embodiments, the another (e.g., non-wild type) or inserted amino acid is A. In some embodiments, the another (e.g., non-wild type) amino acid is Arg (R), Asn (N), Cys (C), Gly (G), lie (I), Leu (L), Pro (P), Ser (S), Trp (W), Tyr (Y), or Val (V). Conventional or naturally occurring amino acids are divided into the following basic groups based on common side-chain properties: (1) non-polar: Norleucine, Met, Ala, Val, Leu, He; (2) polar without charge: Cys, Ser, Thr, Asn, Gin; (3) acidic (negatively charged): Asp, Glu; (4) basic (positively charged): Lys, Arg; and (5) residues that influence chain orientation: Gly, Pro; and (6) aromatic: Trp, Tyr, Phe, His. Conventional amino acids include L or D stereochemistry. In some embodiments, the another (e.g., non-wild type) amino acid is a member of a different group (e.g., an aromatic amino acid is substituted for a non-polar amino acid). Substantial modifications in the biological properties of the polypeptide are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties: (1) Non-polar: Norleucine, Met, Ala, Val, Leu, Ile; (2) Polar without charge: Cys, Ser, Thr, Asn, Gln; (3) Acidic (negatively charged): Asp, Glu; (4) Basic (positively charged): Lys, Arg; (5) Residues that influence chain orientation: Gly, Pro: and (6) Aromatic: Trp, Tyr, Phe, His. In some embodiments, the another (e.g., non-wild type) amino acid is a member of a different group (e.g., a hydrophobic amino acid for a hydrophilic amino acid, a charged amino acid for a neutral amino acid, an acidic amino acid for a basic amino acid, etc.). In some embodiments, the another (e.g., non-wild type) amino acid is a member of the same group (e.g., another basic amino acid, another acidic amino acid, another neutral amino acid, another charged amino acid, another hydrophilic amino acid, another hydrophobic amino acid, another polar amino acid, another aromatic amino acid or another aliphatic amino acid). In some embodiments, the another (e.g., non-wild type) amino acid is an unconventional amino acid. Unconventional amino acids are non-naturally occurring amino acids. Examples of an unconventional amino acid include, but are not limited to, aminoadipic acid, beta-alanine, beta-aminopropionic acid, aminobutyric acid, piperidinic acid, aminocaprioic acid, aminoheptanoic acid, aminoisobutyric acid, aminopimelic acid, citrulline, diaminobutyric acid, desmosine, diaminopimelic acid, diaminopropionic acid, N-ethylglycine. N-ethylaspargine, hyroxylysine, allo-hydroxylysine, hydroxyproline, isodesmosine, allo-isoleucine, N-methylglycine, sarcosine, N-methylisoleucine, N-methylvaline, norvaline, norleucine, orithine, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and amino acids (e.g., 4-hydroxyproline). In some embodiments, one or more amino acid substitutions are introduced into one or more of VP1, VP2 and VP3. In one aspect, a modified capsid protein comprises 1, 2, 3, 4, 5, 6, 7, 8, 9, 10, 11, 12, 13, 14, or 15 conservative or non-conservative substitutions relative to the wild-type polypeptide. In another aspect, the modified capsid polypeptide of the disclosure comprises modified sequences, wherein such modifications can include both conservative and non-conservative substitutions, deletions, and/or additions, and typically include peptides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the corresponding wild-type capsid protein.

In some embodiments, the recombinant AAV vector, rep sequences, cap sequences, and helper functions required for producing the rAAV of the disclosure may be delivered to the packaging host cell using any appropriate genetic element (vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors; a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a different capsid protein, are delivered to the packaging host cell. The selected genetic element may be delivered by any suitable method, including those described herein. The methods used to construct any embodiment of this disclosure are known to those with skill in nucleic acid manipulation and include genetic engineering, recombinant engineering, and synthetic techniques. See. e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). In some embodiments, vectors suitable for use with the present disclosure may be pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Cells may also be transfected with a vector (e.g., helper vector) which provides helper functions to the AAV. The vector providing helper functions may provide adenovirus functions, including, e.g., E1a, E1b, E2a, E40RF6. The sequences ofadenovirus gene providing these functions may be obtained from any known adenovirus serotype, such as serotypes 2, 3, 4, 7, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

An rAAV vector of the disclosure is generated by introducing a nucleic acid sequence encoding an AAV capsid protein, or fragment thereof; a functional rep gene or a fragment thereof; a minigene composed of, at a minimum, AAV inverted terminal repeats (ITRs) and a transgene encoding a complement system polypeptide (e.g. CFI) or a biologically active fragment thereof; and sufficient helper functions to permit packaging of the minigene into the AAV capsid, into a host cell. The components required for packaging an AAV minigene into an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

In some embodiments, such a stable host cell will contain the required component(s) under the control of an inducible promoter. Alternatively, the required component(s) may be under the control of a constitutive promoter. Examples of suitable inducible and constitutive promoters are provided herein, in the discussion below of regulator elements suitable for use with the transgene, i.e., a nucleic acid encoding a complement system polypeptide (e.g. CFI) or biologically active fragment thereof. In still another alternative, a selected stable host cell may contain selected components under the control of a constitutive promoter and other selected components under the control of one or more inducible promoters. For example, a stable host cell may be generated which is derived from cells which contain E1 helper functions under the control of a constitutive promoter, but which contains the rep and/or cap proteins under the control of inducible promoters. Still other stable host cells may be generated by one of skill in the art.

The minigene, rep sequences, cap sequences, and helper functions required for producing the rAAV of the disclosure may be delivered to the packaging host cell in the form of any genetic element which transfers the sequences. The selected genetic element may be delivered by any suitable method known in the art. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, 1993 J. Virol, 70:520-532 and U.S. Pat. No. 5,478,745, among others. These publications are incorporated by reference herein.

Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV.7m8, AAV8, AAV9, AAV10, AAV10, AAV11, AAV12, AAV-DJ, AAV-DJ8, AAV-DJ9, AAVrh8, AAVrh8R or AAVrh10 or other known and unknown AAV serotypes. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.).

Alternatively, the AAV sequences may be obtained through synthetic or other suitable means by reference to published sequences such as are available in the literature or in databases such as, e.g., GenBank, PubMed, or the like.

The minigene is composed of, at a minimum, a transgene encoding a complement system polypeptide (e.g. CFI) or a biologically active fragment thereof, as described above, and its regulatory sequences, and 5 and 3 AAV inverted terminal repeats (ITRs). In one desirable embodiment, the ITRs of AAV serotype 2 are used. However, ITRs from other suitable serotypes may be selected. The minigene is packaged into a capsid protein and delivered to a selected host cell.

In some embodiments, regulatory sequences are operably linked to the transgene encoding a complement system polypeptide (e.g. CFI) or a biologically active fragment thereof. The regulatory sequences may include conventional control elements which are operably linked to the complement system gene, splice variant, or a fragment thereof in a manner which permits its transcription, translation and/or expression in a cell transfected with the vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals: sequences that stabilize cytoplasmic mRNA; sequences that enhance translation efficiency (i.e., Kozak consensus sequence); sequences that enhance protein stability; and when desired, sequences that enhance secretion of the encoded product. Numerous expression control sequences, including promoters, are known in the art and may be utilized.

The regulatory sequences useful in the constructs of the present disclosure may also contain an intron, desirably located between the promoter/enhancer sequence and the gene. In some embodiments, the intron sequence is derived from SV-40, and is a 100 bp mini-intron splice donor/splice acceptor referred to as SD-SA. Another suitable sequence includes the woodchuck hepatitis virus post-transcriptional element. (See, e.g., L. Wang and I. Verma, 1999 Proc. Natl. Acad. Sci., USA, 96:3906-3910). PolyA signals may be derived from many suitable species, including, without limitation SV-40, human and bovine.

Another regulatory component of the rAAV useful in the method of the disclosure is an internal ribosome entry site (IRES). An IRES sequence, or other suitable systems, may be used to produce more than one polypeptide from a single gene transcript (for example, to produce more than one complement system polypeptides). An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the transgene in the rAAV vector.

In some embodiments, expression of the transgene encoding a complement system polypeptide (e.g. CFI) or a biologically active fragment thereof is driven by a separate promoter (e.g., a viral promoter). In certain embodiments, any promoters suitable for use in AAV vectors may be used with the vectors of the disclosure. The selection of the transgene promoter to be employed in the rAAV may be made from among a wide number of constitutive or inducible promoters that can express the selected transgene in the desired ocular cell. Examples of suitable promoters are described below.

Other regulatory sequences useful in the disclosure include enhancer sequences. Enhancer sequences useful in the disclosure include the IRBP enhancer (Nicoud 2007, cited above), immediate early cytomegalovirus enhancer, one derived from an immunoglobulin gene or SV40 enhancer, the cis-acting element identified in the mouse proximal promoter, etc.

Selection of these and other common vector and regulatory elements are well-known and many such sequences are available. See, e.g., Sambrook et al, and references cited therein at, for example, pages 3.18-3.26 and 16, 17-16.27 and Ausubel et al., Current Protocols in Molecular Biology, John Wiley & Sons, New York, 1989).

The rAAV vector may also contain additional sequences, for example from an adenovirus, which assist in effecting a desired function for the vector. Such sequences include, for example, those which assist in packaging the rAAV vector in adenovirus-associated virus particles.

The rAAV vector may also contain a reporter sequence for co-expression, such as but not limited to lacZ. GFP, CFP, YFP, RFP, mCherry, tdTomato, etc. In some embodiments, the rAAV vector may comprise a selectable marker. In some embodiments, the selectable marker is an antibiotic-resistance gene. In some embodiments, the antibiotic-resistance gene is an ampicillin-resistance gene. In some embodiments, the ampicillin-resistance gene is beta-lactamase.

In some embodiments, the rAAV particle is an ssAAV. In some embodiments, the rAAV particle is a self-complementary AAV (sc-AAV) (See, US 2012/0141422 which is incorporated herein by reference). Self-complementary vectors package an inverted repeat genome that can fold into dsDNA without the requirement for DNA synthesis or base-pairing between multiple vector genomes. Because scAAV have no need to convert the single-stranded DNA (ssDNA) genome into double-stranded DNA (dsDNA) prior to expression, they are more efficient vectors. However, the trade-off for this efficiency is the loss of half the coding capacity of the vector, ScAAV are useful for small protein-coding genes (up to −55 kd) and any currently available RNA-based therapy.

rAAV vectors useful in the methods of the disclosure are further described in PCT publication No. WO2015168666 and PCT publication no. WO2014011210, the contents of which are incorporated by reference herein.

In some embodiments, any of the vectors disclosed herein is capable of inducing at least 20%, 50%, 100%, 150%, 200%, 250%, 300%, 400%, 500%, 700%, 900%, 1000%, 1100%, 1500%, or 2000% expression of CFI in a target cell (e.g., an RPE or liver cell) as compared to the endogenous expression of CFI in the target cell. In some embodiments, expression of any of the vectors disclosed herein in a target cell (e.g., an RPE or liver cell) results in at least 20%, 50%, 100%, 150%, 200%, 250%, 300%, 400%, 500%, 700%, 900%, 1000%, 1100%, 1500%, or 2000% levels of CFI activity in the target cell as compared to endogenous levels of CFI activity in the target cell.

In some embodiments, the disclosure provides for a vector, wherein the vector is an AAV2 vector, wherein the vector comprises a CFI-encoding nucleotide sequence that is at least 70%, 75%, 80%, 85%, 90%, 91%, 92%, 93%, 94%, 95%, 96%, 97%, 98%, 99% or 100% identical to the sequence of SEQ ID NO: 34; wherein the vector further comprises a promoter nucleotide sequence that is at least 70%, 75%, 80%, 85%, 90%, 91%, 92%, 93%, 94%, 95%, 96%, 97%, 98%, 99% or 100% identical to the sequence of SEQ ID NO: 19; wherein the vector encodes a CFI protein comprising an A300T mutation as compared to the amino acid sequence of SEQ ID NO: 29; and wherein the CFI protein encoded by the vector is capable of cleaving C3b into iC3b. In some embodiments, the vector comprises one or more ITR sequences flanking the vector portion encoding CFI. In some embodiments, the vector comprises a polyadenylation sequence. In some embodiments, the vector comprises an SV40polyA nucleotide sequence. In some embodiments, the vector comprises a kanamycin-resistance gene. In some embodiments, the vector comprises a nucleotide sequence that is at least 70%, 75%, 80%, 85%, 90%, 91%, 92%, 93%, 94%, 95%, 96%, 97%, 98%, 99%, or 100% identical to the nucleotide sequence of SEQ ID NO: 33, or a functional fragment thereof. In some embodiments, the vector comprises the nucleotide sequence of SEQ ID NO: 33.

Complement System Genes

In the search for causative factors associated with age related macular degeneration, epidemiological and genetic studies have identified numerous common and rare alleles for AMD at or near several complement genes (CFH, C2/CFB, C3, CF1, and C9). Overall, studies have identified that variants near six complement genes (CFH, C2/CFB, C3, CFI, and C9) together accounts for nearly 60% of the AMD genetic risk (Fritsche L G et al. Annu Rev Genomics Hum Genet. 2014; 15:151-71).

Complement system genes (e.g. CFI), splice variants, or fragments thereof are provided as transgenes in the recombinant AAV (rAAV) vectors of the disclosure. The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a target cell (e.g. an ocular cell). The heterologous nucleic acid sequence (transgene) can be derived from any organism. In certain embodiments, the transgene is derived from a human. In certain embodiments, the transgene encodes a mature form of a complement protein. In some embodiments, the transgene encodes a polypeptide comprising an amino acid sequence that is at least 80%, 85%, 90%, 92%, 95%, 97%, 98%, 99%, or 100% identical to the amino acid sequence of SEQ ID NO: 29, or a biologically active fragment thereof. In some embodiments, the transgene encodes a polypeptide comprising an amino acid sequence that is at least 80%, 85%, 90%, 92%, 95%, 97%, 98%, 99%, or 100% identical to the amino acid sequence of SEQ ID NO: 35, or a biologically active fragment thereof. In certain embodiments, the rAAV vector may comprise one or more transgenes.

In some embodiments, the transgene comprises more than one complement system gene, splice variant, or fragments derived from more than one complement system gene. This may be accomplished using a single vector carrying two or more heterologous sequences, or using two or more rAAV vectors each carrying one or more heterologous sequences. In some embodiments, in addition to a complement system gene, splice variant, or fragment thereof, the rAAV vector may also encode additional proteins, peptides. RNA, enzymes, or catalytic RNAs. Desirable RNA molecules include shRNA, tRNA, dsRNA, ribosomal RNA, catalytic RNAs, and antisense RNAs. One example of a useful RNA sequence is a sequence which extinguishes expression of a targeted nucleic acid sequence in the treated subject. The additional proteins, peptides, RNA, enzymes, or catalytic RNAs and the complement factor may be encoded by a single vector carrying two or more heterologous sequences, or using two or more rAAV vectors each carrying one or more heterologous sequences.

In certain aspects, the disclosure provides a recombinant adeno-associated viral (rAAV) vector encoding a human Complement Factor I (CFI) protein or biologically active fragment thereof. In certain embodiments, the vector comprises a nucleotide sequence that is at least 80%, 85%, 90%, 92%, 94%, 95%, 97%, 99% or 100% identical to any of the sequences disclosed herein encoding a CFI protein, or biologically active fragments thereof. In certain embodiments, the vector comprises a nucleotide sequence that is at least 80%, 85%, 90%, 92%, 94%, 95%, 97%, 99% or 100% identical to any of SEQ ID Nos: 1-3, 5 or 34, or biologically active fragments thereof. In certain embodiments, the vector comprises a nucleotide sequence that is at least 80% identical to the nucleotide sequence of any one of SEQ ID NOs: 1-3, 5 or 34, or a fragment thereof. In certain embodiments, the nucleotide sequence is at least 90% identical to the nucleotide sequence of any one of SEQ ID NOs: 1-3, or 34, or a fragment thereof. In certain embodiments, the nucleotide sequence is at least 95% identical to the nucleotide sequence of any one of SEQ ID NOs: 1-3, 5 or 34, or a fragment thereof. In certain embodiments, the nucleotide sequence is the sequence of any one of SEQ ID NOs: 1-3, 5 or 34, or a fragment thereof. In certain embodiments, the vector encodes a CFI protein or biologically active fragment thereof comprising a heavy chain and a light chain. In certain embodiments, the vector encodes a CFI protein or biologically active fragment thereof comprising a FIMAC domain. In certain embodiments, the vector encodes a CF protein or biologically active fragment thereof comprising a Scavenger Receptor Cysteine Rich (SRCR) domain. In certain embodiments, the vector encodes a CFI protein or biologically active fragment thereof comprising at least one LDL receptor Class A domain. In certain embodiments, the vector encodes a CFI protein or biologically active fragment thereof comprising two LDL receptor Class A domains. In certain embodiments, the vector encodes a CFI protein or biologically active fragment thereof comprising a serine protease domain. In certain embodiments, the vector encodes a CFI protein or biologically active fragment thereof comprising a FIMAC domain, a Scavenger Receptor Cysteine Rich (SRCR) domain, and two LDL receptor Class A domains. In certain embodiments, the vector encodes a CFI protein or biologically active fragment thereof capable of cleaving C3b and C4b proteins. In certain embodiments, the vector encodes a CFI protein or biologically active fragment thereof capable of inhibiting the assembly of C3 and C5 convertase enzymes.

In certain embodiments, the vector comprises a nucleotide sequence that is at least 80%, 85%, 90%, 92%, 94%, 95%, 97%, 99% or 100% identical to any of SEQ ID Nos: 1, 7, 14, 16, 18, 20, 22, 24, 26 or 28, or biologically active fragments thereof. In some embodiments, the vector comprises a nucleotide sequence that is at least 80%, 85%, 90%, 92%, 94%, 95%, 97%, 99% or 100% identical to SEQ ID NO: 33.

Exemplary sequences of transgenes are set forth in SEQ ID NOs: 1-3, 5 or 34. In some embodiments, a transgene of the disclosure comprises the nucleic acid sequence set forth in SEQ ID NO: 1. In some embodiments, a transgene of the disclosure comprises the nucleic acid sequence set forth in SEQ ID NO: 2. In some embodiments, a transgene of the disclosure comprises the nucleic acid sequence set forth in SEQ ID NO: 3. In some embodiments, a transgene of the disclosure comprises the nucleic acid sequence set forth in SEQ ID NO: 5. In some embodiments, a transgene of the disclosure comprises a variant of these sequences, wherein such variants can include can include missense mutations, nonsense mutations, duplications, deletions, and/or additions, and typically include polynucleotides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the specific nucleic acid sequences set forth in any one of SEQ ID NOs: 1-3, 5 or 34. In some embodiments, a transgene of the disclosure comprises a variant of these sequences, wherein such variants can include can include missense mutations, nonsense mutations, duplications, deletions, and/or additions, and typically include polynucleotides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the specific nucleic acid sequences set forth in SEQ ID NO: 1. In some embodiments, a transgene of the disclosure comprises a variant of these sequences, wherein such variants can include can include missense mutations, nonsense mutations, duplications, deletions, and/or additions, and typically include polynucleotides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the specific nucleic acid sequences set forth in SEQ ID NO: 2. In some embodiments, a transgene of the disclosure comprises a variant of these sequences, wherein such variants can include can include missense mutations, nonsense mutations, duplications, deletions, and/or additions, and typically include polynucleotides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the specific nucleic acid sequences set forth in SEQ ID NO: 3. In some embodiments, a transgene of the disclosure comprises a variant of these sequences, wherein such variants can include can include missense mutations, nonsense mutations, duplications, deletions, and/or additions, and typically include polynucleotides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the specific nucleic acid sequences set forth in SEQ ID NO: 5. In some embodiments, a transgene of the disclosure comprises a variant of these sequences, wherein such variants can include can include missense mutations, nonsense mutations, duplications, deletions, and/or additions, and typically include polynucleotides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the specific nucleic acid sequences set forth in SEQ ID NO: 34. One of ordinary skill in the art will appreciate that nucleic acid sequences complementary to the nucleic acids, and variants of the nucleic acids are also within the scope of this disclosure. In further embodiments, the nucleic acid sequences of the disclosure can be isolated, recombinant, and/or fused with a heterologous nucleotide sequence. In some embodiments, any of the nucleotides disclosed herein (e.g., SEQ ID Nos: 1-3, 5 or 34) is codon-optimized (e.g., codon-optimized for human expression) In one aspect, a transgene encodes a complement system polypeptide with 1, 2, 3, 4, 5, 6, 7, 8, 9, 10, 11, 12, 13, 14, or 15 amino acid substitutions, deletions, and/or additions relative to the wild-type polypeptide. In some embodiments, a transgene encodes a complement system polypeptide with 1, 2, 3, 4, or 5 amino acid deletions relative to the wild-type polypeptide. In some embodiments, a transgene encodes a polypeptide with 1, 2, 3, 4, or 5 amino acid substitutions relative to the wild-type polypeptide. In some embodiments, a transgene encodes a polypeptide with 1, 2, 3, 4, or 5 amino acid insertions relative to the wild-type polypeptide. Polynucleotides complementary to any of the polynucleotide sequences disclosed herein are also encompassed by the present disclosure. Polynucleotides may be single-stranded (coding or antisense) or double-stranded, and may be DNA (genomic or synthetic), cDNA, or RNA molecules. RNA molecules include mRNA molecules. Additional coding or non-coding sequences may, but need not, be present within a polynucleotide of the present disclosure, and a polynucleotide may, but need not, be linked to other molecules and/or support materials.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene® suite of bioinformatics software (DNASTAR®, Inc., Madison, Wis.), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105: Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity. The transgenes or variants may also, or alternatively, be substantially homologous to a native gene, or a portion or complement thereof. Such polynucleotide variants are capable of hybridizing under moderately stringent conditions to a naturally occurring DNA sequence encoding a complement factor (or a complementary sequence). Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS. As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present disclosure. Further, alleles of the genes comprising the polynucleotide sequences provided herein are within the scope of the present disclosure.

Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The nucleic acids/polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence. In other embodiments, nucleic acids of the disclosure also include nucleotide sequences that hybridize under highly stringent conditions to the nucleotide sequences set forth in any one of SEQ ID NOs: 1-3, 5 or 34, or sequences complementary thereto. One of ordinary skill in the art will readily understand that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. One skilled in the art will appreciate that these variations in one or more nucleotides (up to about 3-5% of the nucleotides) of the nucleic acids encoding a particular protein may exist among members of a given species due to natural allelic variation. Any and all such nucleotide variations and resulting amino acid polymorphisms are within the scope of this disclosure.

The present disclosure further provides oligonucleotides that hybridize to a polynucleotide having the nucleotide sequence set forth in any one of SEQ ID NOs: 1-3, 5 or 34, or to a polynucleotide molecule having a nucleotide sequence which is the complement of a sequence listed above. Such oligonucleotides are at least about 10 nucleotides in length, and preferably from about 15 to about 30 nucleotides in length, and hybridize to one of the aforementioned polynucleotide molecules under highly stringent conditions, i.e., washing in 6×SSC/0.5% sodium pyrophosphate at about 37° C. for about 14-base oligos, at about 48° C. for about 17-base oligos, at about 55° C. for about 20-base oligos, and at about 60° C. for about 23-base oligos. In a preferred embodiment, the oligonucleotides are complementary to a portion of one of the aforementioned polynucleotide molecules. These oligonucleotides are useful for a variety of purposes including encoding or acting as antisense molecules useful in gene regulation, or as primers in amplification of complement system-encoding polynucleotide molecules.

In another embodiment, the transgenes useful herein include reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc. These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

The complement system gene or fragment thereof (e.g. a gene encoding CFI) may be used to correct or ameliorate gene deficiencies, which may include deficiencies in which normal complement system genes are expressed at less than normal levels or deficiencies in which the functional complement system gene product is not expressed. In some embodiments, the transgene sequence encodes a single complement system protein or biologically active fragment thereof. The disclosure further includes using multiple transgenes, e.g., transgenes encoding two or more complement system polypeptides or biologically active fragments thereof. In certain situations, a different transgene may be used to encode different complement proteins or biologically active fragments thereof (e.g. CFI). Alternatively, different complement proteins (e.g. CFI) or biologically active fragments thereof may be encoded by the same transgene. In this case, a single transgene includes the DNA encoding each of the complement proteins (e.g. CFI) or biologically active fragments thereof, with the DNA for each protein or functional fragment thereof separated by an internal ribozyme entry site (IRES). This is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., MX. Donnelly, et al, J. Gen. Virol, 78(Pt 1): 13-21 (January 1997); Furler, S., et al, Gene Ther., 8(11):864-873 (June 2001); Klump H., et al, Gene Ther., 8(10):811-817 (May 2001). This 2A peptide is significantly smaller than an IRES, making it well suited for use when space is a limiting factor.

The regulatory sequences include conventional control elements which are operably linked to the transgene encoding a complement system polypeptide (e.g. CFI) or biologically active fragment thereof in a manner which permits its transcription, translation and/or expression in a cell transfected with the vector or infected with the virus produced as described herein. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters, are known in the art and may be utilized.

The regulatory sequences useful in the constructs provided herein may also contain an intron, desirably located between the promoter/enhancer sequence and the gene. One desirable intron sequence is derived from SV-40, and is a 100 bp mini-intron splice donor/splice acceptor referred to as SD-SA. In some embodiments, the intron comprises the nucleotide sequence of SEQ ID NO: 10, or a codon-optimized or fragment thereof. Another suitable sequence includes the woodchuck hepatitis virus post-transcriptional element. (See, e.g., L. Wang and I. Verma, 1999 Proc. Natl. Acad. Sci., USA. 96:3906-3910). PolyA signals may be derived from many suitable species, including, without limitation SV-40, human and bovine.

Another regulatory component of the rAAV useful in the methods described herein is an internal ribosome entry site (IRES). An IRES sequence, or other suitable systems, may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the transgene in the rAAV vector.

In one embodiment, the AAV comprises a promoter (or a functional fragment of a promoter). The selection of the promoter to be employed in the rAAV may be made from among a wide number of promoters that can express the selected transgene in the desired target cell. In one embodiment, the target cell is an ocular cell. In some embodiments, the target cell is a neuronal cell (i.e., the vector targets neuronal cells). However, in particular embodiments, the target cell is a non-neuronal cell (i.e., the vector does not target neuronal cells). In some embodiments, the target cell is a glial cell, Muller cell, and/or retinal pigment epithelial (RPE) cell. The promoter may be derived from any species, including human. In one embodiment, the promoter is “cell specific”. The term “cell-specific” means that the particular promoter selected for the recombinant vector can direct expression of the selected transgene in a particular cell or ocular cell type. In one embodiment, the promoter is specific for expression of the transgene in photoreceptor cells. In another embodiment, the promoter is specific for expression in the rods and/or cones. In another embodiment, the promoter is specific for expression of the transgene in RPE cells. In another embodiment, the promoter is specific for expression of the transgene in ganglion cells. In another embodiment, the promoter is specific for expression of the transgene in Muller cells. In another embodiment, the promoter is specific for expression of the transgene in bipolar cells. In another embodiment, the promoter is specific for expression of the transgene in ON-bipolar cells. In one embodiment, the promoter is metabotropic glutamate receptor 6 (mGluR6) promoter (see, Vardi et al, mGluR6 Transcripts in Non-neuronal Tissues, J Histochem Cytochem. 2011 December; 59(12): 1076-1086, which is incorporated herein by reference). In another embodiment, the promoter is an enhancer-linked mGluR6 promoter. In another embodiment, the promoter is specific for expression of the transgene in OFF-bipolar cells. In another embodiment, the promoter is specific for expression of the transgene in horizontal cells. In another embodiment, the promoter is specific for expression of the transgene in amacrine cells. In another embodiment, the transgene is expressed in any of the above noted ocular cells. In another embodiment, the promoter is the human G-protein-coupled receptor protein kinase 1 (GRK1) promoter (Genbank Accession number AY327580), In another embodiment, the promoter is the human interphotoreceptor retinoid-binding protein proximal (IRBP) promoter.

In some embodiments, the promoter is of a small size, e.g., under 1000 bp, due to the size limitations of the AAV vector. In some embodiments, the promoter is less than 1000, 900, 800, 700, 600, 500, 400 or 300 bp in size. In particular embodiments, the promoter is under 400 bp. In some embodiments, the promoter is a promoter selected from the CRALBP, EF1a, HSP70, AAT1, ALB, PCK1, CAG, RPE65, or sCBA promoter. In some embodiments, the promoter comprises a nucleotide sequence that is at least 70%, 75%, 80%, 85%, 90%, 95%, 99%, or 100% identical to any one of SEQ ID NOs: 6, 8, 9, 11, 12, 13, 15, 17, 19, 21, 23, 25, 27, or 32 or codon-optimized and/or fragment thereof. In some embodiments, the promoter comprises a nucleotide sequence that is at least 70%, 75%, 80%, 85%, 90%, 95%, 99%, or 100% identical to SEQ ID NO: 6, or codon-optimized and/or fragment thereof. In some embodiments, the promoter comprises a nucleotide sequence that is at least 70%, 75%, 80%, 85%, 90%, 95%, 99%, or 100% identical to SEQ ID NO: 8, or codon-optimized and/or fragment thereof. In some embodiments, the promoter comprises a nucleotide sequence that is at least 70%, 75%, 80%, 85%, 90%, 95%, 99%, or 1001% identical to SEQ ID NO: 9, or codon-optimized and/or fragment thereof. In some embodiments, the promoter comprises a nucleotide sequence that is at least 70%, 75%, 80%, 85%, 90%, 95%, 99%, or 100% identical to SEQ ID NO: 11, or codon-optimized and/or fragment thereof. In some embodiments, the promoter comprises a nucleotide sequence that is at least 70%, 75%, 80%, 85%, 90%, 95%, 99%, or 100% identical to SEQ ID NO: 12, or codon-optimized and/or fragment thereof. In some embodiments, the promoter comprises a nucleotide sequence that is at least 70%, 75%, 800%, 85%, 90%, 95%, 99%, or 100% identical to SEQ ID NO: 13, or codon-optimized and/or fragment thereof. In some embodiments, the promoter comprises a nucleotide sequence that is at least 70%, 75%, 80%, 85%, 90%, 95%, 99%, or 100% identical to SEQ ID NO: 15, or codon-optimized and/or fragment thereof. In some embodiments, the promoter comprises a nucleotide sequence that is at least 70%, 75%, 80%, 85%, 90° %, 95%, 99%, or 100% identical to SEQ ID NO: 17, or codon-optimized and/or fragment thereof. In some embodiments, the promoter comprises a nucleotide sequence that is at least 70%, 75%, 80%, 85%, 90%, 95%, 99%, or 100% identical to SEQ ID NO: 19, or codon-optimized and/or fragment thereof. In some embodiments, the promoter comprises a nucleotide sequence that is at least 70%, 75%, 80%, 85%, 90%, 95%, 99%, or 100% identical to SEQ ID NO: 21, or codon-optimized and/or fragment thereof. In some embodiments, the promoter comprises a nucleotide sequence that is at least 70%, 75%, 80%, 85%, 90%, 95%, 99%, or 100% identical to SEQ ID NO: 23, or codon-optimized and/or fragment thereof. In some embodiments, the promoter comprises a nucleotide sequence that is at least 70%, 75%, 80%, 85%, 90%, 95%, 99%, or 100% identical to SEQ ID NO: 25, or codon-optimized and/or fragment thereof. In some embodiments, the promoter comprises a nucleotide sequence that is at least 70%, 75%, 80%, 85%, 90%, 95%, 99%, or 100% identical to SEQ ID NO: 27, or codon-optimized and/or fragment thereof. In some embodiments, the promoter comprises a nucleotide sequence that is at least 70%, 75%, 80%, 85%, 90%, 95%, 99%, or 100% identical to SEQ ID NO: 32, or codon-optimized and/or fragment thereof. In some embodiments, the promoter comprises the nucleotide sequence of any one of SEQ ID NOs: 6, 8, 9, 11, 12, 13, 15, 17, 19, 21, 23, 25, 27, or 32 or codon-optimized and/or fragment thereof. In some embodiments, the promoter is associated with strong expression in the liver. In some embodiments, the promoter is an AAT1, ALB or PCK1 promoter (e.g., a promoter having the nucleotide sequence of SEQ ID NO: 13, 15 or 27. In some embodiments, the promoter is greater than 1000 bp in size. In some embodiments, the promoter is greater than 1000, 1100, 1200, 1300, 1400, 1500, or 1600 bp in size. In some embodiments, the promoter is approximately 1600 bp in size (plus or minus 50 nucleotides). In some embodiments, the promoter is a 1.6 Kb CBA promoter (e.g., a promoter having the nucleotide sequence of SEQ ID NO: 6 or a codon-optimized and/or fragment thereof). In some embodiments, if the gene to be expressed in the AAV vector is CFI (e.g., a gene comprising the nucleotide sequence of any one of SEQ ID NOs: 1-3, 5 or 34, or a codon-optimized and/or fragment thereof), then the promoter is greater than 1000, 1100, 1200, 1300, 1400, 1500, or 1600 bp in size. In some embodiments, if the gene to be expressed in the AAV vector is CFI (e.g., a gene comprising the nucleotide sequence of any one of SEQ ID NOs: 1-3, 5 or 34, or a codon-optimized and/or fragment thereof), then the promoter is approximately 1600 bp in size (plus or minus 50 nucleotides). In some embodiments, if the gene to be expressed in the AAV vector is CFI (e.g., a gene comprising the nucleotide sequence of any one of SEQ ID NOs: 1-3, 5 or 34, or a codon-optimized and/or fragment thereof), then the promoter is a 1.6 Kb CBA promoter (e.g., a promoter having the nucleotide sequence of SEQ ID NO: 6 or a codon-optimized and/or fragment thereof).

In another embodiment, the promoter is the native promoter for the gene to be expressed. Useful promoters include, without limitation, the rod opsin promoter, the red-green opsin promoter, the blue opsin promoter, the cGMP-β-phosphodiesterase promoter, the mouse opsin promoter (Beltran et al 2010 cited above), the rhodopsin promoter (Mussolino et al, Gene Ther, July 2011, 18(7):637-45); the alpha-subunit of cone transducin (Morrissey et al, BMC Dev, Biol, January 2011, 11:3); beta phosphodiesterase (PDE) promoter; the retinitis pigmentosa (RP1) promoter (Nicoud et al, J. Gene Med, December 2007, 9(12): 1015-23); the NXNL2/NXNLI promoter (Lambard et al, PLoS One, October 2010, 5(10):e13025), the RPE65 promoter; the retinal degeneration slow/peripherin 2 (Rds/perph2) promoter (Cai et al, Exp Eye Res. 2010 August; 91(2): 186-94); and the VMD2 promoter (Kachi et al, Human Gene Therapy, 2009 (20:31-9)). Each of these documents is incorporated by reference herein. In one embodiment, the promoter is of a small size, under 1000 bp, due to the size limitations of the AAV vector. In another embodiment, the promoter is under 400 bp.

In certain embodiments, any promoters suitable for use in AAV vectors may be used with the vectors of the disclosure. Examples of suitable promoters include constitutive promoters such as a CMV promoter (optionally with the CMV enhancer), RSV promoter (optionally with the RSV enhancer), SV40 promoter, MoMLV promoter, CB promoter, the dihydrofolate reductase promoter, the chicken β-actin (CBA) promoter, CBA/CAG promoter, and the immediate early CMV enhancer coupled with the CBA promoter, or a EF1a promoter, etc. In some embodiments a cell- or tissue-specific promoter is utilized (e.g., a rod, cone, or ganglia derived promoter). In certain embodiments, the promoter is small enough to be compatible with the disclosed constructs, e.g., the CB promoter. Preferably, the promoter is a constitutive promoter. In another embodiment, the promoter is cell-specific. The term “cell-specific” means that the particular promoter selected for the recombinant vector can direct expression of the selected transgene in a particular ocular cell type. In one embodiment, the promoter is specific for expression of the transgene in photoreceptor cells. In another embodiment, the promoter is specific for expression in the rods and cones. In another embodiment, the promoter is specific for expression in the rods. In another embodiment, the promoter is specific for expression in the cones. In another embodiment, the promoter is specific for expression of the transgene in RPE cells. In another embodiment, the transgene is expressed in any of the above noted ocular cells.

Other useful promoters include transcription factor promoters including, without limitation, promoters for the neural retina leucine zipper (Nr1), photoreceptor-specific nuclear receptor Nr2e3, and basic-leucine zipper (bZIP). In one embodiment, the promoter is of a small size, under 1000 bp, due to the size limitations of the AAV vector. In another embodiment, the promoter is under 400 bp.

Other regulatory sequences useful herein include enhancer sequences. Enhancer sequences useful herein include the IRBP enhancer (Nicoud 2007, cited above), immediate early cytomegalovirus enhancer, one derived from an immunoglobulin gene or SV40 enhancer, the cis-acting element identified in the mouse proximal promoter, etc.

Selection of these and other common vector and regulatory elements are conventional and many such sequences are available. See, e.g., Sambrook et al, and references cited therein at, for example, pages 3.18-3.26 and 16.17-16.27 and Ausubel et al., Current Protocols in Molecular Biology, John Wiley & Sons, New York, 1989). It is understood that not all vectors and expression control sequences will function equally well to express all of the transgenes as described herein. However, one of skill in the art may make a selection among these, and other, expression control sequences to generate the rAAV vectors of the disclosure.

Production of rAAV Vectors

Numerous methods are known in the art for production of rAAV vectors, including transfection, stable cell line production, and infectious hybrid virus production systems which include adenovirus-AAV hybrids, herpesvirus-AAV hybrids (Conway, J E et al., (1997). Virology 71(11):8780-8789) and baculovirus-AAV hybrids, rAAV production cultures for the production of rAAV virus particles all require. 1) suitable host cells, including, for example, human-derived cell lines such as HcLa. A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a transgene (such as a transgene encoding a complement system polypeptide (e.g. CFI) or a biologically active fragment thereof) flanked by at least one AAV ITR sequence; and 5) suitable media and media components to support rAAV production. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors.

The rAAV particles can be produced using methods known in the art. See, e.g., U.S. Pat. Nos. 6,566,118; 6,989,264; and 6,995,006. In practicing the disclosure, host cells for producing rAAV particles include mammalian cells, insect cells, plant cells, microorganisms and yeast. Host cells can also be packaging cells in which the AAV rep and cap genes are stably maintained in the host cell or producer cells in which the AAV vector genome is stably maintained. Exemplary packaging and producer cells are derived from 293, A549 or HeLa cells. AAV vectors are purified and formulated using standard techniques known in the art.

Recombinant AAV particles are generated by transfecting producer cells with a plasmid (cis-plasmid) containing a rAAV genome comprising a transgene flanked by the 145 nucleotide-long AAV ITRs and a separate construct expressing the AAV rep and CAP genes in trans. In addition, adenovirus helper factors such as E1A, E1B, E2A, E40RF6 and VA RNAs, etc. may be provided by either adenovirus infection or by transfecting a third plasmid providing adenovirus helper genes into the producer cells. Packaging cell lines suitable for producing adeno-associated viral vectors may be readily accomplished given readily available techniques (see e.g., U.S. Pat. No. 5,872,005). The helper factors provided will vary depending on the producer cells used and whether the producer cells already carry some of these helper factors.

In some embodiments, rAAV particles may be produced by a triple transfection method, such as the exemplary triple transfection method provided infra. Briefly, a plasmid containing a rep gene and a capsid gene, along with a helper adenoviral plasmid, may be transfected (e.g., using the calcium phosphate method) into a cell line, and virus may be collected and optionally purified.

In some embodiments, rAAV particles may be produced by a producer cell line method, such as the exemplary producer cell line method provided infra (see also (referenced in Martin et al., (2013) Human Gene Therapy Methods 24:253-269). Briefly, a cell line (e.g., a HeLa cell line) may be stably transfected with a plasmid containing a rep gene, a capsid gene, and a promoter-transgene sequence. Cell lines may be screened to select a lead clone for rAAV production, which may then be expanded to a production bioreactor and infected with an adenovirus (e.g., a wild-type adenovirus) as helper to initiate rAAV production. Virus may subsequently be harvested, adenovirus may be inactivated (e.g., by heat) and/or removed, and the rAAV particles may be purified.

In some aspects, a method is provided for producing any rAAV particle as disclosed herein comprising (a) culturing a host cell under a condition that rAAV particles are produced, wherein the host cell comprises (i) one or more AAV package genes, wherein each said AAV packaging gene encodes an AAV replication and/or encapsidation protein; (ii) a rAAV pro-vector comprising a nucleic acid encoding a therapeutic polypeptide and/or nucleic acid as described herein flanked by at least one AAV ITR, and (iii) an AAV helper function; and (b) recovering the rAAV particles produced by the host cell. In some embodiments, said at least one AAV ITR is selected from the group consisting of AAV ITRs are AAV1, AAV2, AAV3, AAV4, AAV5, AAV6, AAV7, AAV.7m8, AAV8, AAVrh8, AAVrh8R, AAV9, AAV10, AAVrh10, AAV11, AAV12, AAV2R471A, AAV DJ, a goat AAV, bovine AAV, or mouse AAV or the like. In some embodiments, the encapsidation protein is an AAV2 encapsidation protein.

Suitable rAAV production culture media of the present disclosure may be supplemented with serum or serum-derived recombinant proteins at a level of 0.5-20 (v/v or w/v). Alternatively, as is known in the art, rAAV vectors may be produced in serum-free conditions which may also be referred to as media with no animal-derived products. One of ordinary skill in the art may appreciate that commercial or custom media designed to support production of rAAV vectors may also be supplemented with one or more cell culture components know in the art, including without limitation glucose, vitamins, amino acids, and or growth factors, in order to increase the titer of rAAV in production cultures.

rAAV production cultures can be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors, rAAV vector production cultures may also include suspension-adapted host cells such as HeLa, 293, and SF-9 cells which can be cultured in a variety of ways including, for example, spinner flasks, stirred tank bioreactors, and disposable systems such as the Wave bag system.

rAAV vector particles of the disclosure may be harvested from rAAV production cultures by lysis of the host cells of the production culture or by harvest of the spent media from the production culture, provided the cells are cultured under conditions known in the art to cause release of rAAV particles into the media from intact cells, as described more fully in U.S. Pat. No. 6,566,118). Suitable methods of lysing cells are also known in the art and include for example multiple freeze/thaw cycles, sonication, microfluidization, and treatment with chemicals, such as detergents and/or proteases.

In a further embodiment, the rAAV particles are purified. The term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

In some embodiments, the rAAV production culture harvest is clarified to remove host cell debris. In some embodiments, the production culture harvest is clarified by filtration through a series of depth filters including, for example, a grade DOHC Millipore Millistak+HC Pod Filter, a grade A1HC Millipore Millistak+HC Pod Filter, and a 0.2μιη Filter Opticap XL 10 Millipore Express SHC Hydrophilic Membrane filter. Clarification can also be achieved by a variety of other standard techniques known in the art, such as, centrifugation or filtration through any cellulose acetate filter of 0.2μιη or greater pore size known in the art.

In some embodiments, the rAAV production culture harvest is further treated with Benzonase® to digest any high molecular weight DNA present in the production culture. In some embodiments, the Benzonase® digestion is performed under standard conditions known in the art including, for example, a final concentration of 1-2.5 units/ml of Benzonase® at a temperature ranging from ambient to 37° C. for a period of 30 minutes to several hours.

rAAV particles may be isolated or purified using one or more of the following purification steps: equilibrium centrifugation; flow-through anionic exchange filtration; tangential flow filtration (TFF) for concentrating the rAAV particles; rAAV capture by apatite chromatography; heat inactivation of helper virus; rAAV capture by hydrophobic interaction chromatography; buffer exchange by size exclusion chromatography (SEC); nanofiltration; and rAAV capture by anionic exchange chromatography, cationic exchange chromatography, or affinity chromatography. These steps may be used alone, in various combinations, or in different orders. In some embodiments, the method comprises all the steps in the order as described below. Methods to purify rAAV particles are found, for example, in Xiao et al., (1998) Journal of Virology 72:2224-2232; U.S. Pat. Nos. 6,989,264 and 8,137,948; and WO 2010/148143.

Pharmaceutical Compositions

Also provided herein are pharmaceutical compositions comprising an rAAV particle comprising a transgene encoding a complement system polypeptide (e.g. CFI) or a biologically active fragment thereof and/or therapeutic nucleic acid, and a pharmaceutically acceptable carrier. The pharmaceutical compositions may be suitable for any mode of administration described herein; for example, by intravitreal administration.

In some embodiments, the composition comprises a polypeptide (or a nucleic acid encoding a polypeptide) that processes (e.g., cleaves) the complement system polypeptide encoded by the transgene in the rAAV. However, in particular embodiments, the composition does not comprise a polypeptide (or a nucleic acid encoding a polypeptide) that processes (e.g., cleaves) the complement system polypeptide encoded by the transgene in the rAAV. In particular embodiments, the composition does not comprise a polypeptide (or a nucleic acid encoding a polypeptide) that processes (e.g., cleaves) a CFI polypeptide encoded by the transgene in the rAAV. In some embodiments, the processing polypeptide is a protease. In some embodiments, the protease is furin.

In some embodiments, gene therapy protocols for retinal diseases, such as LCA, retinitis pigmentosa, and age-related macular degeneration may involve the localized delivery of the vector to the cells in the retina. The cells that will be the treatment target in these diseases are either the photoreceptor cells in the retina or the cells of the RPE underlying the neurosensory retina. Delivering gene therapy vectors to these cells may involve injection into the subretinal space between the retina and the RPE. In some embodiments, the disclosure provides methods to deliver rAAV gene therapy vectors encoding a complement system polypeptide (e.g. CFI) or a biologically active fragment thereof to cells of the retina.

In some embodiments, the pharmaceutical compositions comprising a rAAV described herein and a pharmaceutically acceptable carrier is suitable for administration to a human subject. Such carriers are well known in the art (see, e.g., Remington's Pharmaceutical Sciences, 15th Edition, pp. 1035-1038 and 1570-1580). In some embodiments, the pharmaceutical compositions comprising a rAAV described herein and a pharmaceutically acceptable carrier is suitable for ocular injection. In some embodiments, the pharmaceutical composition is suitable for intravitreal injection. In some embodiments, the pharmaceutical composition is suitable for subretinal delivery. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

In one embodiment, the recombinant AAV containing the desired transgene encoding a complement system polypeptide (e.g. CFI) or a biologically active fragment thereof and constitutive or tissue or cell-specific promoter for use in the target ocular cells as detailed above is formulated into a pharmaceutical composition intended for subretinal or intravitreal injection. Such formulation involves the use of a pharmaceutically and/or physiologically acceptable vehicle or carrier, particularly one suitable for administration to the eye, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20. In another embodiment, the pharmaceutically acceptable carrier comprises a surfactant, such as perfluorooctanc (Perfluoron liquid).

In certain embodiments of the methods described herein, the pharmaceutical composition described above is administered to the subject by subretinal injection. In other embodiments, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Routes of administration may be combined, if desired. In certain embodiments, the pharmaceutical compositions of the disclosure are administered after administration of an initial loading dose of the complement system protein.

In some embodiments, any of the vectors/pharmaceutical compositions disclosed herein are administered to a patient such that they target cells of any one or more layers or regions of the retina or macula. For example, the compositions disclosed herein target cells of any one or more layers of the retina, including the inner limiting membrane, the nerve fiber layer, the ganglion cell layer (GCL), the inner plexiform layer, the inner nuclear layer, the outer plexiform layer, the outer nuclear layer, the external limiting membrane, the layer of rods and cones, or the retinal pigment epithelium (RPE). In some embodiments, the compositions disclosed herein target glial cells of the GCL, Muller cells, and/or retinal pigment epithelial cells. In some embodiments, the compositions disclosed herein targets cells of any one or more regions of the macula including, for example, the umbo, the foveolar, the foveal avascular zone, the fovea, the parafovea, or the perifovea. In some embodiments, the route of administration does not specifically target neurons. In some embodiments, the route of administration is chosen such that it reduces the risk of retinal detachment in the patient (e.g., intravitreal rather than subretinal administration). In some embodiments, intravitreal administration is chosen if the vector/composition is to be administered to an elderly adult (e.g., at least 60 years of age). In particular embodiments, any of the vectors/pharmaceutical compositions disclosed herein are administered to a subject intravitreally. Procedures for intravitreal injection are known in the art (see, e.g., Peyman, G. A., et al. (2009) Retina 29(7):875-912 and Fagan, X. J. and Al-Qureshi, S. (2013) Clin. Experiment. Ophthalmol. 41(5):500-7). Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as Povidone-Iodine (BETADINE®). A similar solution may also be used to clean the eyelid, eyelashes, and any other nearby tissues {e.g., skin). Any suitable anesthetic may be used, such as lidocaine or proparacaine, at any suitable concentration. Anesthetic may be administered by any method known in the art, including without limitation topical drops, gels or jellies, and subconjuctival application of anesthetic. Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limus in pseudophakic or aphakic patients, and 3.5-4 mm from the limbus in phakic patients. The patient may look in a direction opposite the injection site. During injection, the needle may be inserted perpendicular to the sclera and pointed to the center of the eye. The needle may be inserted such that the tip ends in the vitreous, rather than the subretinal space. Any suitable volume known in the art for injection may be used. After injection, the eye may be treated with a sterilizing agent such as an antiobiotic. The eye may also be rinsed to remove excess sterilizing agent.

Furthermore, in certain embodiments it is desirable to perform non-invasive retinal imaging and functional studies to identify areas of specific ocular cells to be targeted for therapy. In these embodiments, clinical diagnostic tests are employed to determine the precise location(s) for one or more subretinal injection(s). These tests may include ophthalmoscopy, electroretinography (ERG) (particularly the b-wave measurement), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc.

These, and other desirable tests, are described in Intemational Patent Application No. PCT/US2013/022628. In view of the imaging and functional studies, in some embodiments, one or more injections are performed in the same eye in order to target different areas of retained bipolar cells. The volume and viral titer of each injection is determined individually, as further described below, and may be the same or different from other injections performed in the same, or contralateral, eye. In another embodiment, a single, larger volume injection is made in order to treat the entire eye. In one embodiment, the volume and concentration of the rAAV composition is selected so that only a specific region of ocular cells is impacted. In another embodiment, the volume and/or concentration of the rAAV composition is a greater amount, in order reach larger portions of the eye, including non-damaged ocular cells.

The composition may be delivered in a volume of from about 0.1 μL to about 1 mL, including all numbers within the range, depending on the size of the area to be treated, the viral titer used, the route of administration, and the desired effect of the method. In one embodiment, the volume is about 50 μL. In some embodiments, the volume is between 25-100 μL. In some embodiments, the volume is between 40-60 μL. In another embodiment, the volume is about 70 μL. In a preferred embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 L In another embodiment, the volume is about 300 L In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. An effective concentration of a recombinant adeno-associated virus carrying a nucleic acid sequence encoding the desired transgene under the control of the cell-specific promoter sequence desirably ranges from about 107 and 10¹³ vector genomes per milliliter (vg/mL) (also called genome copies/mL (GC/mL)). The rAAV infectious units are measured as described in S. K. McLaughlin et al, 1988 J. Virol., 62: 1963, which is incorporated herein by reference. Preferably, the concentration in the retina is from about 1.5×10⁹ vg/mL to about 1.5×10¹² vg/mL, and more preferably from about 1.5×10⁹ vg/mL to about 1.5×10¹¹ vg/mL. In certain preferred embodiments, the effective concentration is about 2.5×10¹⁰ vg to about 1.4×10¹¹. In one embodiment, the effective concentration is about 1.4×10⁸ vg/mL. In one embodiment, the effective concentration is about 3.5×10¹⁰ vg/mL. In another embodiment, the effective concentration is about 5.6×10¹¹ vg/mL. In another embodiment, the effective concentration is about 5.3×10¹² vg/mL. In yet another embodiment, the effective concentration is about 1.5×10¹² vg/mL. In another embodiment, the effective concentration is about 1.5×10¹³ vg/mL. In one embodiment, the effective dosage (total genome copies delivered) is from about 10⁷ to 10¹³ vector genomes. It is desirable that the lowest effective concentration of virus be utilized in order to reduce the risk of undesirable effects, such as toxicity, retinal dysplasia and detachment. Still other dosages and administration volumes in these ranges may be selected by the attending physician, taking into account the physical state of the subject, preferably human, being treated, the age of the subject, the particular ocular disorder and the degree to which the disorder, if progressive, has developed. For extra-ocular delivery, the dosage will be increased according to the scale-up from the retina. Intravenous delivery, for example may require doses on the order of 1.5×10¹³ vg/kg.

Pharmaceutical compositions useful in the methods of the disclosure are further described in PCT publication No. WO2015168666 and PCT publication no. WO2014011210, the contents of which are incorporated by reference herein.

Methods of Treatment Prophylaxis

Described herein are various methods of preventing, treating, arresting progression of or ameliorating the ocular disorders and retinal changes associated therewith. Generally, the methods include administering to a mammalian subject in need thereof, an effective amount of a composition comprising a recombinant adeno-associated virus (AAV) described above, carrying a transgene encoding a complement system polypeptide (e.g. CFI) or a biologically active fragment thereof under the control of regulatory sequences which express the product of the gene in the subject's ocular cells, and a pharmaceutically acceptable carrier. Any of the AAV described herein are useful in the methods described below.

In some embodiments, gene therapy protocols for retinal diseases, such as LCA, retinitis pigmentosa, and age-related macular degeneration may involve the localized delivery of the vector to the cells in the retina. The cells that will be the treatment target in these diseases are either the photoreceptor cells in the retina or the cells of the RPE underlying the neurosensory retina. Delivering gene therapy vectors to these cells may involve injection into the subretinal space between the retina and the RPE. In some embodiments, the disclosure provides methods to deliver rAAV gene therapy vectors comprising a complement system gene or a fragment thereof to cells of the retina.

In a certain aspect, the disclosure provides a method of treating a subject having age-related macular degeneration (AMD), comprising the step of administering to the subject any of the vectors of the disclosure. In some embodiments, the subject has drusen deposits and/or geographic atrophy. In certain embodiments, the vectors are administered at a dose between 2.5×10¹⁰ vg and 1.4×10¹³ vg/per eye in about 50 μl to about 100 μl. In certain embodiments, the vectors are administered at a dose between 1.0×10¹¹ vg and 1.5×10¹³ vg/per eye in about 50 μl to about 100 μl. In certain embodiments, the vectors are administered at a dose between 1.0×10¹¹ vg and 1.5×10¹² vg/per eye in about 50 μl to about 100 μl. In certain embodiments, the vectors are administered at a dose of about 1.4×10¹² vg/per eye in about 50 μl to about 100 μl. In certain embodiments, the vectors are administered at a dose of 1.4×10¹² vg/per eye in about 50 μl to about 100 μl. In certain embodiments, the pharmaceutical compositions of the disclosure comprise a pharmaceutically acceptable carrier. In certain embodiments, the pharmaceutical compositions of the disclosure comprise PBS. In certain embodiments, the pharmaceutical compositions of the disclosure comprise pluronic. In certain embodiments, the pharmaceutical compositions of the disclosure comprise PBS, NaCl and pluronic. In certain embodiments, the vectors are administered by intravitreal injection in a solution of PBS with additional NaCl and pluronic.

In some embodiments, any of the vectors of the present disclosure used according to the methods disclosed herein is capable of inducing at least 5%, 10%, 20%, 50%, 100%, 150%, 200%, 250%, 300%, 400%, 500%, 700%, 900%, 1000%, 1100%, 1500%, or 2000% expression of CFI in a target cell disclosed herein (e.g., an RPE or liver cell) as compared to the endogenous expression of CFI in the target cell. In some embodiments, expression of any of the vectors disclosed herein in a target cell disclosed herein (e.g., an RPE or liver cell) results in at least 5%, 10%, 20%, 50%, 100%, 150%, 200%, 250%, 300%, 400%, 500%, 700%, 900%, 1000%, 1100%, 1500%, or 2000% levels of CFI activity in the target cell as compared to endogenous levels of CFI activity in the target cell.

In some embodiments, any of the vectors disclosed herein is administered to cell(s) or tissue(s) in a test subject. In some embodiments, the cell(s) or tissue(s) in the test subject express less CFI, or less functional CFI, than expressed in the same cell type or tissue type in a reference control subject or population of reference control subjects. In some embodiments, the reference control subject is of the same age and/or sex as the test subject. In some embodiments, the reference control subject is a healthy subject, e.g., the subject does not have a disease or disorder of the eye. In some embodiments, the reference control subject does not have a disease or disorder of the eye associated with activation of the complement cascade. In some embodiments, the reference control subject does not have macular degeneration. In some embodiments, the reference control subject does not have drusen deposits or geographic atrophy. In some embodiments, the eye or a specific cell type of the eye (e.g., cells in the foveal region) in the test subject express at least 95%, 90%, 80%, 70%, 60%, 50%, 40%, 30%, 20%, 10%, 5%, or 1% less CF or functional CF as compared to the levels in the reference control subject or population of reference control subjects. In some embodiments, the eye or a specific cell type of the eye (e.g., cells in the foveal region) in the test subject express CFI protein having any of the CFI mutations disclosed herein. In some embodiments, the eye or a specific cell type of the eye (e.g., cells in the fovcal region) in the reference control subject do not express a CFI protein having any of the CFI mutations disclosed herein. In some embodiments, expression of any of the vectors disclosed herein in the cell(s) or tissue(s) of the test subject results in an increase in levels of CFI protein or functional CFI protein. In some embodiments, expression of any of the vectors disclosed herein in the cell(s) or tissue(s) of the test subject results in an increase in levels of CFI protein or functional CFI protein such that the increased levels are within 90%, 80%, 70%, 60%, 50%, 40%, 30%, 20%, 10%, 5%, or 1% of, or are the same as, the levels of CFI protein or functional CFI protein expressed by the same cell type or tissue type in the reference control subject or population of reference control subjects. In some embodiments, expression of any of the vectors disclosed herein in the cell(s) or tissue(s) of the test subject results in an increase in levels of CFI protein or functional CFI protein, but the increased levels of CFI protein or functional CFI protein do not exceed the levels of CFI protein or functional CFI protein expressed by the same cell type or tissue type in the reference control subject or population of reference control subjects. In some embodiments, expression of any of the vectors disclosed herein in the cell(s) or tissue(s) of the test subject results in an increase in levels of CFI protein or functional CFI protein, but the increased levels of CFI protein or functional CF protein exceed the levels of CFI protein or functional CFI protein by no more than 1%, 5%, 10%, 20%, 25%, 30%, 40%, 50%. 60%, 70%, 80%, 90% or 100% of the levels expressed by the same cell type or tissue type in the reference control subject or population of reference control subjects.

In some embodiments, any of the treatment and/or prophylactic methods disclosed herein are applied to a subject. In some embodiments, the subject is a mammal. In some embodiments, the subject is a human. In some embodiments, the human is an adult. In some embodiments, the human is an elderly adult. In some embodiments, the human is at least 40, 45, 50, 55, 60, 65, 70, 75, 80, 85, 90, or 95 years of age. In particular embodiments, the human is at least 60 or 65 years of age.

In some embodiments, any of the treatment and/or prophylactic methods disclosed herein is for use in treatment of a patient having one or more mutations that causes macular degeneration (AMD) or that increases the likelihood that a patient develops AMD. In some embodiments, any of the treatment and/or prophylactic methods disclosed herein is for use in treatment of a patient having one or more mutations that causes atypical hemolytic uremic syndrome (aHUS) or that increases the likelihood that a patient develops aHUS. In some embodiments, the one or more mutations are in the patient's CFI gene. In some embodiments, the one or more mutations are in the patient's CFH gene. In some embodiments, the one or more mutations are in both the patient's CFH and CFI genes. In some embodiments, the subject has a loss-of-function mutation in the subject's CFH gene. In some embodiments, the subject has a loss-of-function mutation in the subject's CFI gene.

In some embodiments, the disclosure provides a method for treating a subject having a disease or disorder, wherein the subject has one or more CFI mutations. A subject “has” a CFI mutation if DNA from a sample (e.g., a blood sample or a sample from the patient's eye) from the subject is determined to carry one or more CFI mutations. In some embodiments, any of the methods disclosed herein are for treating a subject in whom it has been determined has one or more CFI mutations. In some embodiments, the presence or absence of any of the CFI mutations disclosed herein is determined by genetic testing.

In some embodiments, any of the treatment and/or prophylactic methods disclosed herein is for use in treatment of a patient having one or more mutations in the patient's CFI gene. In some embodiments, the patient has a mutation in one or more of the FIMAC, CD5, L1, L1-Ca binding, L1-disulfid bond, L2, L2-Ca binding, serine protease, or serine protease active site domains. In some embodiments, the patient has one or more mutations in the disulphide bond sites in the CFI protein. In some embodiments, the mutation is one or more of the mutations selected from the group consisting of: E548Q, V412M, A431T, A431S, K441R, P553S, A240G, A258T, G119R, G261D, R202I, T300A, T203I, V152M, R317W, G287R, E554V, I340T, G162D, P50A, Y206N, D310E, H418L, p.(Tyr411Stop), p.(Arg187Stop), R474Q, Y459S, R187Q, R339Q, G263V, p.(Arg339Stop), D477H, p.(Ile357Met), P64L, E109A, G125R, N177I, F198L, S221Y, D224N, C229R, V230M, G248E, G280D, A356P, V20I, Y369S, W374C, R389H, W399R, C467R, G487C, I492L, G500R, R502C, W541*, V543A, Q580*, V355M, I578T, R474*, R406H, D44N, p.(Arg406Cys), D403N, I416L, G328R, G512S, p.(Gly542Ser), p.(Cys106Arg), V127A, p.(Ile55Phe), H40R, C54R, C54*, V184M, G362A, Q462H, N536K, R317Q, p.(His 183Arg), p.(IIe306Val), p.(Gly342Glu), p.(Asp429Glu), R448H, D519N, S493R, R448C, K338Q, G104R, C259R, G372S, A360V, E290A, V213F, F13V, Y514Ter, V396A, E303Q, H401Q, I306T, E479G, c.772+1G>T, F498L, Y411H, S24T, C255Y, R168S, Q228R, V469I, Q250K, Y241C, G232V, G248R, G110R, E109K, N422D, C550R, G242AfsTer9, R345G, N428MfsTer5, C550WfsTer17, V341E, N428S, H334P, W51R, A452S, T72S, T72S, V558I, E445G, C444Y, L351I, G261S, M138I, A563S, G263AfsTer37, K142E, c.658+2T>C, G205D, T197A, G188V, A378V, L376P, C365Y, M147V, Q161Ter, G439R, G269S, R201 S, P576S, Y65H, c.907+1G>Aâ€, Y22C, I407T, M204V, A384T, G516V, R336G, F139V, L4H, K117E, V489I, P402L, G547R, A346T, S326P, I126T, D283G, S298F, loss of Met1, Ter584QextTer24, C521Y, R168G, S457P, A423E, L34V, A452T, K442E, N245K, D173N, K267E, S146R, E302K, G295V, V299L, K111N, S113N, F17V, Q391E, H14L, T3941, c.659-2A>G, A511V, E303K, D398G, Ter584KextTer24, V583A, A163T, Hi18Q, A309S, T23I, G473R, V530I, E26Ter, K497N, S496C, S496T, L491R, V412E, F417S, S570G, D465G, E124K, D567V, G557D, E548G, W546G, V543I, N464K, P463A, N564S, K561E, E445D, C444G, D443H, E434KfsTer2, 1430T, I244S, I244V, c328+1G>A, R345Q, S175F, N331KfsTer46, C327R, K130I, Q260E, P96S, I140T, T137I, D135G, K69E, G57D, G371V, G367A, N279S, Y276C, G269C, E190D, T300A, G261D, N151S, R406H, V152M, G362A, E554V, S570T, I340T, K441R, T203I, Y206N, G328R, T107A, P553S, G287R, N70T, P50A, R406C, R187Q, G119R, 1429+1G>C, D477H, N177I, V129A, I55V, W399R, G500R, I492L, R339Ter, I357M, R474Q, D44N, D403N, R474Ter, R317W, G512S, R339Q, A356P, R187Ter, 1416L, R317L, R389H, I306V, D224Y, R317Q, A258T, Q580Tet, H418L, I578T, G542S, P64L, C106R, Y369S, Q462H, A240G, H183R, R502G, H40R or G162D. In particular embodiments, the mutation is any one of the mutations selected from the group consisting of: G119R, L131R, V152M, G162D, R187Y, R187T, T203I, A240G, A258T, G287R, A300T, R317W, R339Q, V412M, and P553S. In some embodiments, any of the CFI mutant amino acid positions described herein correspond to the wildtype amino acid CFI sequence of SEQ ID NO: 29.

In some embodiments, the patient has any one of the following mutations: P553S, K441R, R339Q, R339Ter, R317Q, R317W, A300T, G287R, G261D, A258T, A240G, T203I, R187Q, R187Ter, G162D, V152M or G119R. In some embodiment, the patient has a P553S mutation. In some embodiments, the patient has a K441R mutation. In some embodiments, the patient has an R339Q mutation. In some embodiments, the patient has an R339Ter mutation. In some embodiments, the patient has an R317Q mutation. In some embodiments, the patient has an R317W mutation. In some embodiments, the patient has an A300T mutation. In some embodiments, the patient has a G287R mutation. In some embodiments, the patient has a G261D mutation. In some embodiments, the patient has an A258T mutation. In some embodiments, the patient has an A240G mutation. In some embodiments, the patient has a T203I mutation. In some embodiments, the patient has an R187Q mutation. In some embodiments, the patient has an R187Ter mutation. In some embodiments, the patient has a G162D mutation. In some embodiments, the patient has a V152M mutation. In some embodiments, the patient has a G119R mutation.

Documents referencing some of the CFI mutations disclosed herein include: Saksens et al., 2016, JAMA Ophthalmol, 134(3):287-293; Nilsson et al., 2010, Eur. J. Immunol., 40:172-185; Nilsson et al., 2007, Molecular Immunol., 44:1835-1844; Kavanagh et al., 2015, Human Molecular Genetics, 24(13):3861-3870; Kavanagh et al., 2008, Molecular Immunology, 45:95-105; Geerlings et al., 2018, Clinical Genetics, 94:330-338; Geerlings et al., 2017, JAMA Ophthalmol, 135(1): 39-46; Fritsche et al., 2016, Nat. Genet., 48(2):134-143; Cayci et al., 2012, Pediatr Nephrol., 27:2327-2331; Caprioli et al., 2006, Blood, 108(4):1267-1279: Bienaime et al., 2010, Kidney International, 77:334-349: Alexander et al., 2014, Molecular Vision, 20:1253-57; Seddon et al., 2013, Nat. Genet., 45(11):1366-1370; and Van de Ven et al., 2013, Nat. Genet., 45(7):813-819.

In some embodiments, any of the CFI mutant amino acid positions described herein correspond to the wildtype amino acid CFI sequence of SEQ ID NO: 29.

In some embodiments, the patient is homozygous for any of the mutations disclosed herein. In some embodiments, the patient is heterozygous for any of the mutations disclosed herein. In particular embodiments, the patient expresses a mutant CFI protein, wherein the mutant CFI protein has reduced CFI activity as compared to a wildtype CFI protein (e.g., a CFI protein having the amino acid sequence of SEQ ID NO: 29). In some embodiments, the CFI activity is the ability to cleave C3b to iC3b. In some embodiments, if the mutant CFI protein were tested in a functional assay, the mutant CFI protein would display reduced CFI activity as compared to a wildtype CFI protein (e.g., a CFI protein having the amino acid sequence of SEQ ID NO: 29). In some embodiments, the functional assay tests the ability of CFI to cleave C3b to iC3b (see, e.g., Example 7 for a representative assay testing the ability of CFI to cleave C3b to iC3b). Examples of CFI mutants associated with reduced CFI activity (e.g., reduce ability to cleave C3b to iC3b) include G119R, A240G or P553S CFI mutants. See. e.g., Example 7.

In some embodiments, any of the treatment and/or prophylactic methods disclosed herein is for use in treatment of a patient having one or more mutations in the patient's CFH gene. In some embodiments, the patient has a mutation in one or more of the pre-SCR1 or any of the SCR1-SCR20 domains. In some embodiments, the patient has a mutation in one or more of the transition regions between SCRs. In some embodiments, the mutation is one or more of the mutations selected from the group consisting of: H402Y, G69E, D194N, W314C, A806T, Q950H, p.Ile184fsX, p.Lys204fsX, c.1697-17_-8del, A161S, A173G, R175Q, V62I, V1007L, S890I, S193L, I216T, A301Nfs*25, W379R, Q400K, Q950H, T956M, R1210C, N1050Y, E936D, Q408X, R1078S, c.350+6T->G, R567G, R53C, R53H, R2T, A892V, R567G, I221V, S159N, P562H, F960S, R303W, R303Q, K666N, G1194D, P258L, G650V, D130N, S58A, R166W, R232Q, R127H, K1202N, G397Stop, Stop450R, R830W, I622L, T732M, S884Y, L24V, Y235H, K527N, R582H, C973Y, V1089M, E123G, T291S, R567K, E625Stop, N802S, N1056K, R1203W, Q1076E, P26S, T46A, T91S, C129Y, R166Q, E167Q, R175P, C192F, W198*, V206M, G218*, M239T, Y277*, C325Y, R341H, R364L, P384R, C431S, D454A, A473V, P503A, N516K, I551T, H699R, F717L, W978R, P981S, A1010V, W1037*, P1051L, I1059T, Q1143E, R1206H, T1227I, L24V, H169R, R257H, K410E, V609I, D619N, A892V, G1002R, G278S, T30*, I32Stop, R78G, Q81P, V11E, W134R, P139S, M162V, E189Stop, K224Del, K224Del, A307A, H332Y, S411T, C448Y, L479Stop, R518T, T519A, C536R, C564P, C569Stop, L578Stop, P621T, C623S, C630W, E635D, K670T, Q672Q, C673Y, C673S, S714Stop, S722*, C733Y, V737V, E762Stop, N774Stop, R780I, G786*, M823T, V835L, E847V, E850K, C853R, C853T, C864S, C870R, H878H, I881L, E889Stop, H893R, Y899Stop, Y899D, C915S, C915Stop, W920R, Q925Stop, C926F, Y951H, C959Y, P968*, I970V, T987A, N997T, G1011*, T1017I, Y1021F, C1043R, T1046T, V1054I, V1060A, V1060L, C1077W, T1097W, T1097T, D119G, D119N, P1130L, V1134G, E1135R, E1137L, E1139Stop, Y1142D, Y1142C, C1152S, W1157R, P1161T, C1163T, P1166L, V1168E, V1168Stop, I1169L, E1172Stop, Y1177C, R1182S, W1183L, W1183R, W1183L, W1183Stop, W1183C, T1184R, T1184A, K1186H, K1188Del, L1189R, L1189F, S1191L, S1191W, E1195Stop, V1197A, E1198A, E1198Stop, F1199S, V1200L, G1204E, L1207R, S1211P, R1215Q, R1215G, T1216Del, C1218R, Y1225*, P1226S, L3V, H821Y, E954del, G255E, T1038R, V383A, V641A, P213A, I221V, E229K, R2T, R1072G, G967E, N819S, V579F, G19K, A18S, K834E, T504M, R662I, P668L, G133R, I184T, L697F, H1165Y, G1110A, pIle808_Gln809del, 1760L, T447R, I808M, I868M, L765F, N767S, R567G, K768N, S209L, Q628K, D214Y, N401D, I216K, Q464R, I777V, E229D, M823I, R232Ter, S266L, P260S, E23G, C80Y, R78T, R582H, N638D, N638S, P258L, L3F, R257H, G240R, G69R, D855N, M11I, K472N, Q840H, E850K, Y899H, T645M, M805V, K919T, E201G, V407A, I907L, T914K, H332R, V144M, S652G, D195N, C146S, P661R, E677Q, V482I, T34R, A421T, R281G, C509Y, K666N, P440S, C442G, N607D, A425V, G667E, P440L, I49V, R387G, E625K, E625Ter, T135S, P43S, K283E, I124V, T36V, I563T, G350E, D619G, T321I, T286A, P384L, T739N, M515L, V158A, G727R, T724K, F717L, M162V, C178R, G700R, A161T, F176L, R295S, F298Y, G297S, P300L, R1040K, V552L, T310I, T531A, G928D, Ter386RextTer 69â€, Q1143K, Y534C, P981L, K308N, D538E, R1215Ter, E105V, T1017I, N1050I, P935S, Y951H, T1097M, D947H, E961D, G962S, G964E, I970V, R1072T, P1114L, S1122T, F960C, R1074C, R1182T, R1074L, S884Y, S890T, V8371, V941F, V158I, D748V, I216T, H371N, L750F, P418T, M432V, D693N, A746E, VI 11E, c.2237-2A>G, P982S, V579A, E591D, V579I, V65I, P418S, Y1067C, D772N, V72L, E189K, A1027P, D798N, N61D, P384S, N521S, P1068S, E395K, N774S, H577R, E833K, K6E, H337R, R444C, L741F, Y42F, D288E, S705F, R1040G, D214H, N757D, I861M, G848E, P923S, E201K, E902A, R303Q, G366E, D538H, K82R, E721K, Y1008H, R1074P, A806S, Q807R, C389Y, H764Y, K867N, P392T, L394M, E456K, F459L, Y398C, E570K, D214N, I574V, I574T, G631C, T880I, V865F, V576A, N776S, P633S, N22D, P634A, N822I, R885S, R232L, E635D, R778K, L827V, C267R, Y779C, R582C, L77S, R257C, Y327H, N75K, L74F, S836T, Y243H, c.1519+5_1519+8delGT . . . , K507Q, A892S, I15T, P924L, A14V, N842K, G894R, G894E, Y271C, C9W, T504R, V683M, L385Pheâ€, S898R, Q408H, G409S, T34K, E648G, I412V, E338D, P799S, G480E, D798E, D195Y, R341C, D485H, D485G, K598Q, Y420H, P599T, N434H, R441T, C431G, V149A, V349I, T679A, P43T, G45D, R662G, T519I, L121P, P364L, P621A, H373Y, D538MfsTer14, H371P, T544A, T131A, R166G, V177I, V177A, R729S, F717V, N718S, S991G, L98I, Y1016Ter, T1217del, M1001T, K1004E, A1010T, G1011D, T1017A, T1031A, L1125F, R1203G, L1214M, W1096DfsTer20, H939N F960L, D966H, M1064I, E1071K, N1095K, T1106A, G1107E, C1109W, P1111S, V1197I, Y1075F, S1079N, P1080S, E1082G, or Sto1232, In particular embodiments, the mutation is one or more of the mutations selected from the group consisting of: R2T, L3V, R53C, R53H, S58A, G69E, D90G, R175Q, S193L, I216T, I221V, R303W, H402Y, Q408X, P503A, G650V, R1078S, and R1210C. In some embodiments, any of the CFH mutant amino acid positions described herein correspond to the wildtype amino acid CFH sequence of SEQ ID NO: 30.

In some embodiments, the subject is a subject in whom it has been determined has any one or more of any of the CFI mutations disclosed herein.

In some embodiments, any of the vectors disclosed herein are for use in treating a renal disease or complication. In some embodiments, the renal disease or complication is associated with AMD in the patient. In some embodiments, the renal disease or complication is associated with aHUS in the patient. In some embodiments, the vector administered for treating a renal disease or complication comprises a promoter that is associated with strong expression in the liver. In some embodiments, the promoter is an AAT1 (SERPINEA1), ALB or PCK1 promoter (e.g., a promoter comprising the nucleotide sequence of any one of SEQ ID Nos: 13, 15 or 27, respectively).

The retinal diseases described above are associated with various retinal changes. These may include a loss of photoreceptor structure or function; thinning or thickening of the outer nuclear layer (ONL); thinning or thickening of the outer plexiform layer (OPL); disorganization followed by loss of rod and cone outer segments; shortening of the rod and cone inner segments; retraction of bipolar cell dendrites; thinning or thickening of the inner retinal layers including inner nuclear layer, inner plexiform layer, ganglion cell layer and nerve fiber layer; opsin mislocalization; overexpression of neurofilaments; thinning of specific portions of the retina (such as the fovea or macula); loss of ERG function; loss of visual acuity and contrast sensitivity; loss of optokinetic reflexes; loss of the pupillary light reflex; and loss of visually guided behavior. In one embodiment, a method of preventing, arresting progression of or ameliorating any of the retinal changes associated with these retinal diseases is provided. As a result, the subject's vision is improved, or vision loss is arrested and/or ameliorated.

In a particular embodiment, a method of preventing, arresting progression of or ameliorating vision loss associated with an ocular disorder in the subject is provided. Vision loss associated with an ocular disorder refers to any decrease in peripheral vision, central (reading) vision, night vision, day vision, loss of color perception, loss of contrast sensitivity, or reduction in visual acuity.

In another embodiment, a method of targeting one or more type(s) of ocular cells for gene augmentation therapy in a subject in need thereof is provided. In another embodiment, a method of targeting one or more type of ocular cells for gene suppression therapy in a subject in need thereof is provided. In yet another embodiment, a method of targeting one or more type of ocular cells for gene knockdown/augmentation therapy in a subject in need thereof is provided. In another embodiment, a method of targeting one or more type of ocular cells for gene correction therapy in a subject in need thereof is provided. In still another embodiment, a method of targeting one or more type of ocular cells for neurotropic factor gene therapy in a subject in need thereof is provided.

In any of the methods described herein, the targeted cell may be an ocular cell. In one embodiment, the targeted cell is a glial cell. In one embodiment, the targeted cell is an RPE cell. In another embodiment, the targeted cell is a photoreceptor. In another embodiment, the photoreceptor is a cone cell. In another embodiment, the targeted cell is a Muller cell. In another embodiment, the targeted cell is a bipolar cell. In yet another embodiment, the targeted cell is a horizontal cell. In another embodiment, the targeted cell is an amacrine cell. In still another embodiment, the targeted cell is a ganglion cell. In still another embodiment, the gene may be expressed and delivered to an intracellular organelle, such as a mitochondrion or a lysosome.

As used herein “photoreceptor function loss” means a decrease in photoreceptor function as compared to a normal, non-diseased eye or the same eye at an earlier time point. As used herein, “increase photoreceptor function” means to improve the function of the photoreceptors or increase the number or percentage of functional photoreceptors as compared to a diseased eye (having the same ocular disease), the same eye at an earlier time point, a non-treated portion of the same eye, or the contralateral eye of the same patient. Photoreceptor function may be assessed using the functional studies described above and in the examples below, e.g., ERG or perimetry, which are conventional in the art.

For each of the described methods, the treatment may be used to prevent the occurrence of retinal damage or to rescue eyes having mild or advanced disease. As used herein, the term “rescue” means to prevent progression of the disease to total blindness, prevent spread of damage to uninjured ocular cells, improve damage in injured ocular cells, or to provide enhanced vision. In one embodiment, the composition is administered before the disease becomes symptomatic or prior to photoreceptor loss. By symptomatic is meant onset of any of the various retinal changes described above or vision loss. In another embodiment, the composition is administered after disease becomes symptomatic. In yet another embodiment, the composition is administered after initiation of photoreceptor loss. In another embodiment, the composition is administered after outer nuclear layer (ONL) degeneration begins. In some embodiments, it is desirable that the composition is administered while bipolar cell circuitry to ganglion cells and optic nerve remains intact.

In another embodiment, the composition is administered after initiation of photoreceptor loss. In yet another embodiment, the composition is administered when less than 90% of the photoreceptors are functioning or remaining, as compared to a non-diseased eye. In another embodiment, the composition is administered when less than 80% of the photoreceptors are functioning or remaining. In another embodiment, the composition is administered when less than 70% of the photoreceptors are functioning or remaining. In another embodiment, the composition is administered when less than 60% of the photoreceptors are functioning or remaining. In another embodiment, the composition is administered when less than 50% of the photoreceptors are functioning or remaining. In another embodiment, the composition is administered when less than 40% of the photoreceptors are functioning or remaining. In another embodiment, the composition is administered when less than 30% of the photoreceptors are functioning or remaining. In another embodiment, the composition is administered when less than 20% of the photoreceptors are functioning or remaining. In another embodiment, the composition is administered when less than 10% of the photoreceptors are functioning or remaining. In one embodiment, the composition is administered only to one or more regions of the eye. In another embodiment, the composition is administered to the entire eye.

In another embodiment, the method includes performing functional and imaging studies to determine the efficacy of the treatment. These studies include ERG and in vivo retinal imaging, as described in the examples below. In addition visual field studies, perimetry and microperimetry, pupillometry, mobility testing, visual acuity, contrast sensitivity, color vision testing may be performed.

In yet another embodiment, any of the above described methods is performed in combination with another, or secondary, therapy. The therapy may be any now known, or as yet unknown, therapy which helps prevent, arrest or ameliorate any of the described retinal changes and/or vision loss. In one embodiment, the secondary therapy is encapsulated cell therapy (such as that delivering Ciliary Neurotrophic Factor (CNTF)). See, Sieving, P. A. et al, 2006. Proc Natl Acad Sci USA, 103(10):3896-3901, which is hereby incorporated by reference. In another embodiment, the secondary therapy is a neurotrophic factor therapy (such as pigment epithelium-derived factor, PEDF; ciliary neurotrophic factor 3; rod-derived cone viability factor (RdCVF) or glial-derived neurotrophic factor). In another embodiment, the secondary therapy is anti-apoptosis therapy (such as that delivering X-linked inhibitor of apoptosis, XIAP). In yet another embodiment, the secondary therapy is rod derived cone viability factor 2. The secondary therapy can be administered before, concurrent with, or after administration of the rAAV described above.

In some embodiments, any of the vectors or compositions disclosed herein is administered to a subject in combination with any of the other vectors or compositions disclosed herein. In some embodiments, any of the vectors or compositions disclosed herein is administered to a subject in combination with another therapeutic agent or therapeutic procedure. In some embodiments, the additional therapeutic agent is an anti-VEGF therapeutic agent (e.g., such as an anti-VEGF antibody or fragment thereof such as ranibizumab, bevacizumab or aflibercept), a vitamin or mineral (e.g., vitamin C, vitamin E, lutein, zeaxanthin, zinc or copper), omega-3 fatty acids, and/or Visudyne™. In some embodiments, the other therapeutic procedure is a diet having reduced omega-6 fatty acids, laser surgery, laser photocoagulation, submacular surgery, retinal translocation, and/or photodynamic therapy.

In some embodiments, any of the vectors disclosed herein is administered to a subject in combination with an additional agent needed for processing and/or improving the function of the protein encoded by the vector/composition. For example, if the vector comprises a CFI gene, the vector may be administered to a patient in combination with an antibody (or a vector encoding that antibody) that potentiates the activity of an endogenous CFH protein. Examples of such antibodies are found in WO2016/028150, which is incorporated herein in its entirety. In some embodiments, the vector is administered in combination with an additional polypeptide (or a vector encoding that additional polypeptide), wherein the additional polypeptide is capable of processing the protein encoded by the vector, e.g., processing an encoded precursor protein into its mature form. In some embodiments, the processing protein is a protease (e.g., a furin protease). For example, if the vector encoded a precursor CFI protein, in some embodiments, it may be advantageous to administer that vector in combination with a protease (e.g., a furin protease), or a vector encoding that protease, in combination with the CFI-encoding vector. However, in alternative embodiments, any of the vectors disclosed herein is not administered with any additional vector encoding a processing polypeptide (or a vector encoding that processing polypeptide). For example, in some embodiments, the disclosure contemplates methods of administering a vector encoding a CFI protein, wherein the vector is not administered in combination with a processing polypeptide (e.g., a furin) or a vector encoding a processing polypeptide (e.g., a furin). In some embodiments, the disclosure contemplates a composition comprising any of the vectors disclosed herein, wherein that composition does not include any additional processing polypeptide (e.g., furin) or vector encoding a processing polypeptide (e.g., furin). In some embodiments, the disclosure contemplates administering a vector encoding a CFI protein to a patient, wherein the method contemplates the patient utilizing endogenous sources of a processing polypeptide (e.g., furin) to process the CFI protein to its mature form. That is, in some embodiments, the compositions disclosed herein are capable of being processed to active CFI. In some embodiments, the compositions of the present disclosure, used according to the methods disclosed herein, are capable of being processed to active CFI.

Kits

In some embodiments, any of the vectors disclosed herein is assembled into a pharmaceutical or diagnostic or research kit to facilitate their use in therapeutic, diagnostic or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

### EXAMPLES

The disclosure now being generally described, it will be more readily understood by reference to the following examples, which are included merely for purposes of illustration of certain embodiments and embodiments of the present disclosure, and are not intended to limit the disclosure.

### Example 1: Construction of AAV Vectors

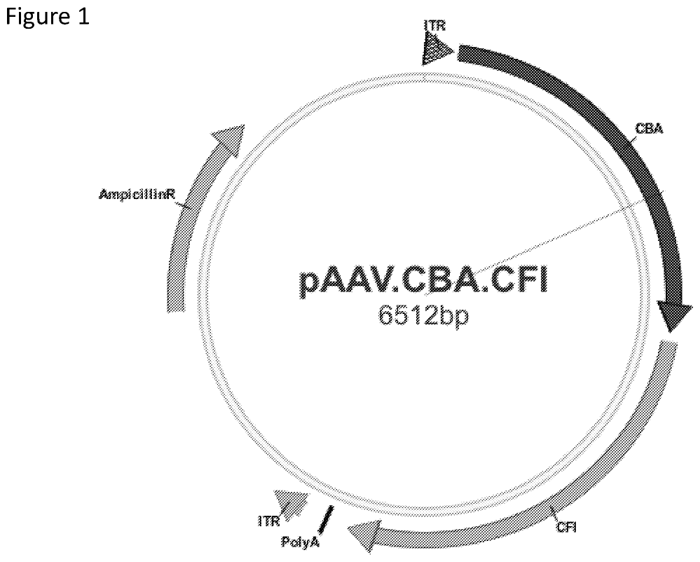

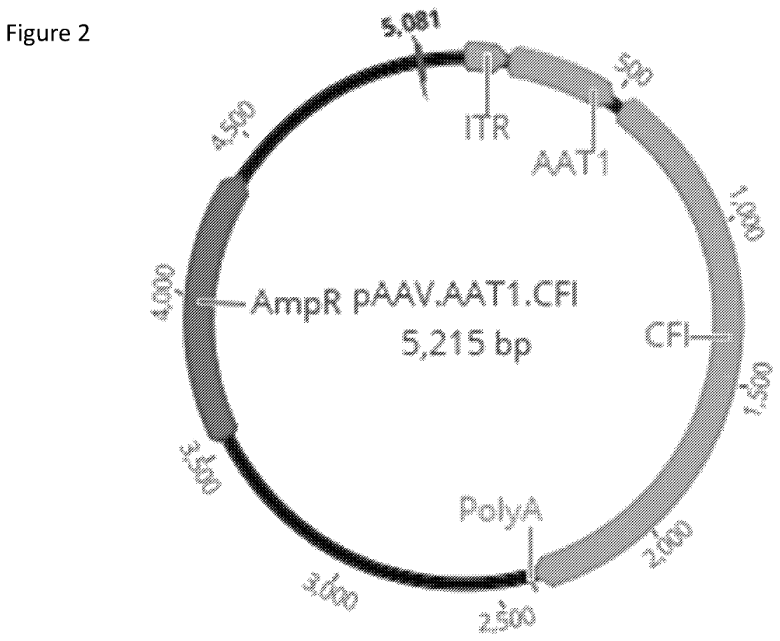

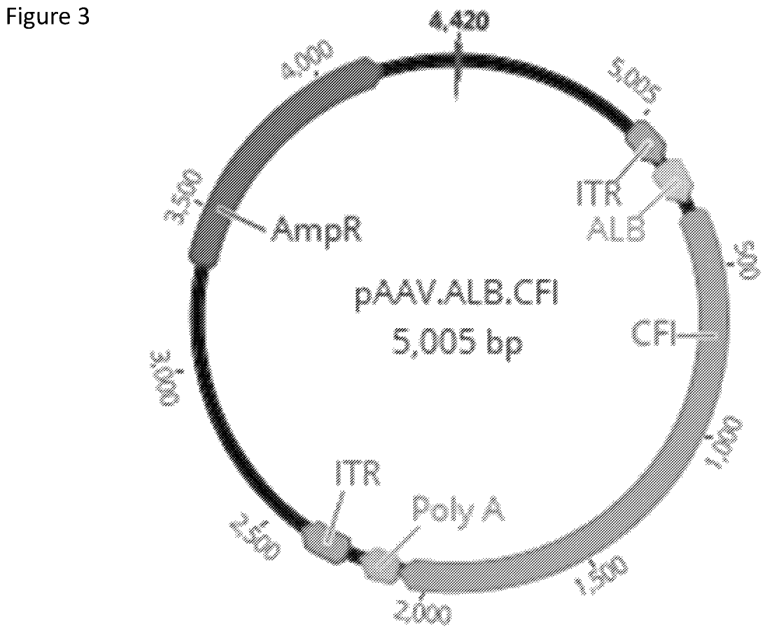

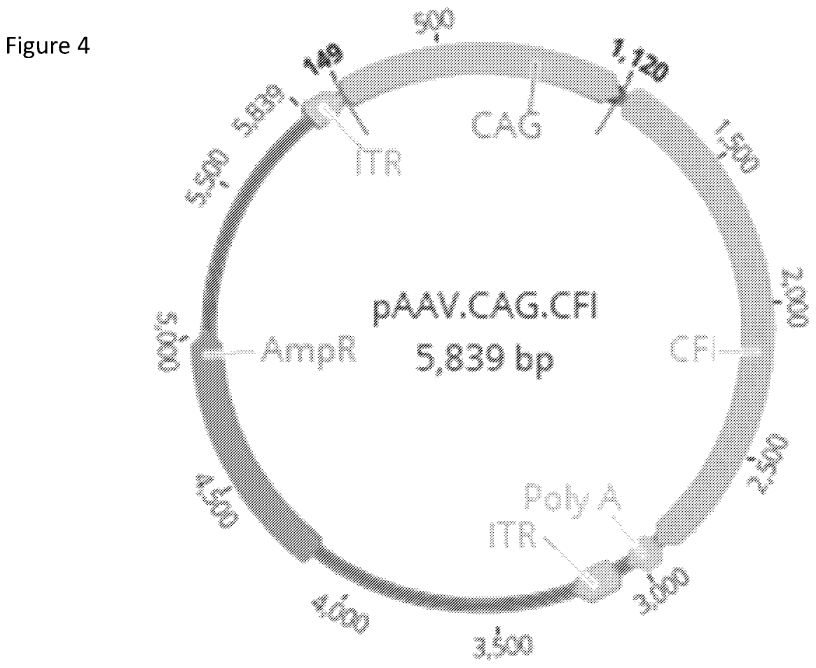

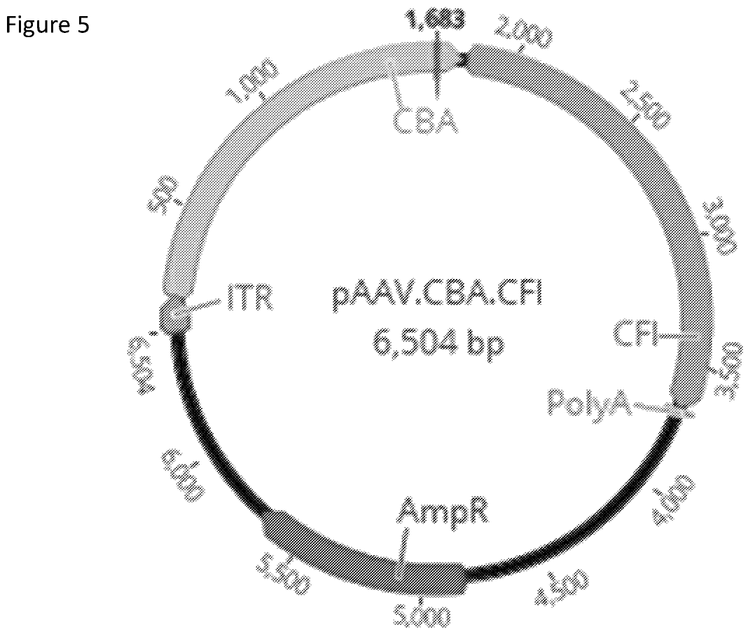

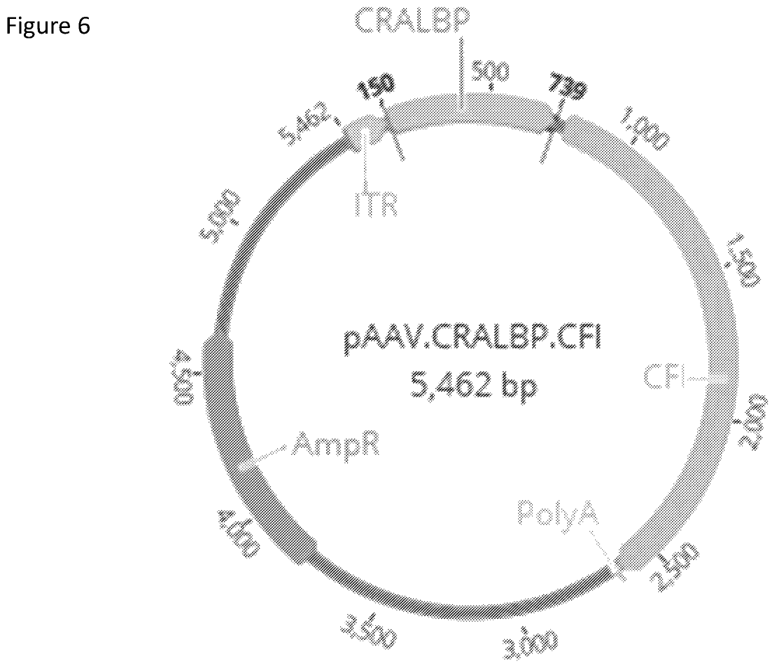

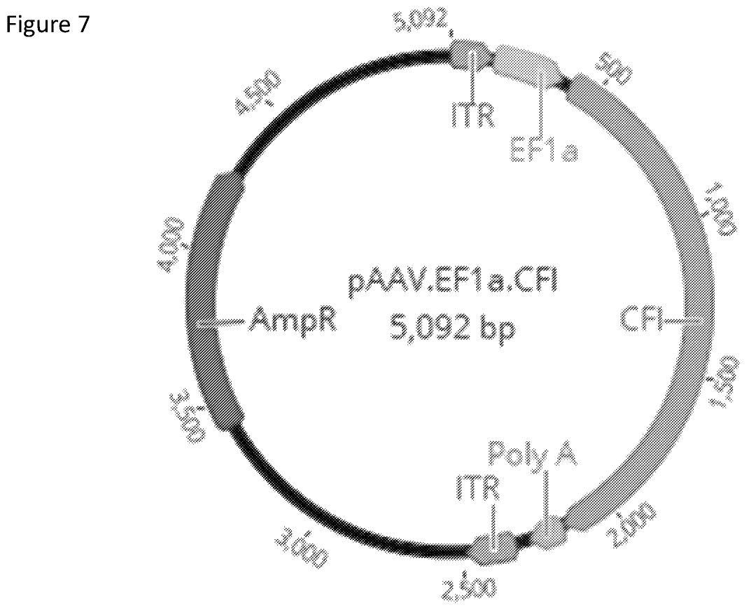

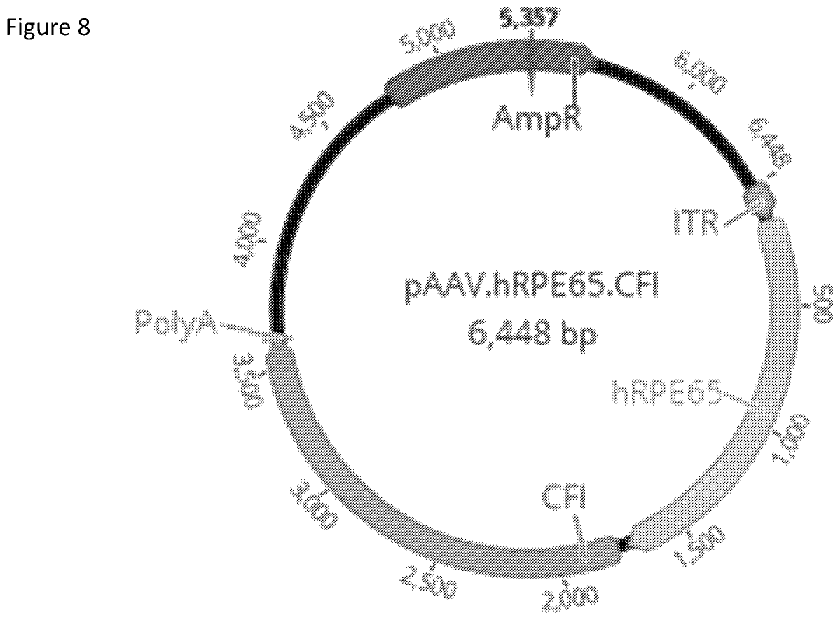

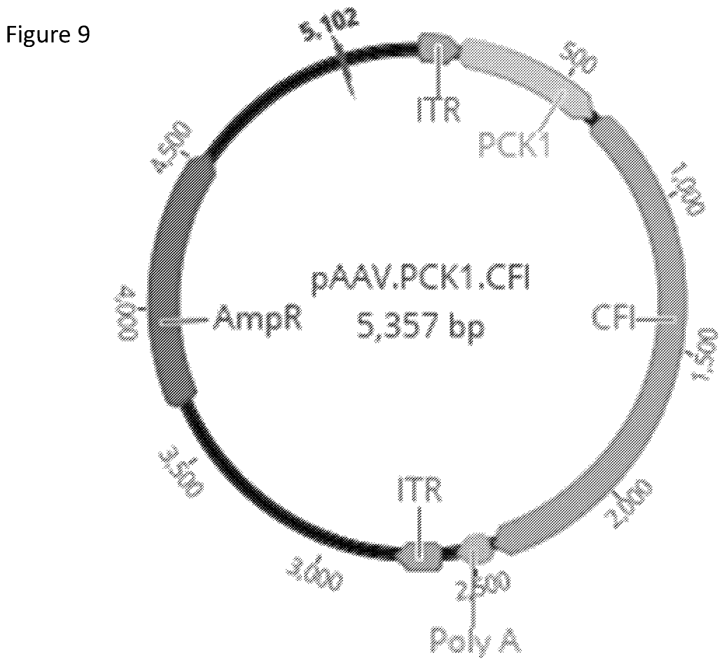

AAV2 vectors were designed comprising either codon-optimized or non-codon-optimized CFI or CF sequences in combination with a variety of different promoters and, in some cases, SV40 introns. FIGS. 1-9 and 19 show vector maps of the different vectors generated. A table is provided below outlining the gene included in the cassette, the promoter included, the Figure laying out the construct map, and the sequence associated with the vector.

| | | | | Construct |
|---|---|---|---|---|
| | | | | Sequence |
| Construct Name | Transgene | Promoter | FIG. | (SEQ ID NO) |
| pAAV.CBA.CFI | CFI | CBA | 1 | 7 |
| pAAV-AAT1-CFI | CFI | AAT1 | 2 | 14 |
| pAAV-ALB-CFI | CFI | ALB | 3 | 16 |
| pAAV-CAG-CFI | CFI | CAG | 4 | 18 |
| pAAV-CBA-CFI | CFI | CBA | 5 | 20 |
| pAAV-CRALBP-CFI | CFI | CRALBP | 6 | 22 |
| pAAV-EF1a-CFI | CFI | EF1a | 7 | 24 |
| pAAV-RPE65-CFI | CFI | RPE65 | 8 | 26 |
| pAAV-PCK1-CFI | CFI | PCK1 | 9 | 28 |
| pAAV-CBA-CFI | CFI | CBA | 19 | 33 |

### Ability of AAV.CFI Vectors to Transduce Cells and Regulate Complement Activity:

Any of the CFI vectors disclosed above will be first tested in vitro in ARPE19 cells via transfection and evaluated for expression of the human CFI protein in both cell pellets and in the supernatant. Techniques like Western blot will be used for protein detection and quantification. Quantitative Real time PCR will be used for determining mRNA expression levels. To determine the proper processing of CFI, western blots will be performed to discern both the light and heavy chains of the protein. A co-factor assay will be run to ensure the functionality of the processed protein. Regulation of complement activity will be tested in a cell culture model of blue light irradiation of A2E-laden retinal pigment epithelial cells as described in van der Burght et al, Acta Ophthalmol, 2013. Briefly, ARPE-19 cell line is grown to confluence and cultured in standard media plus or minus 10 uM A2E for 4 weeks. RPE are irradiated with blue light. Media is replaced with PBS plus calcium, magnesium and 5.5 mM glucose and cells are irradiated with blue light (430+/−30 nm) for 0, 5 or 10 minutes. RPE cells are incubated with appropriately-complement depleted human serum+/− and transfected with the AAV.CFI vectors. Immunoreactivity of RPE with cell surface markers, CD46, CD55 and CD59 and C3 and MAC deposition will be assessed by fluorescent microscopy or western blot. Levels of iC3b (cleavage product of C3) will be measured by Western Blot or ELISA.

After evaluation in ARPE19 cells, the AAV.CFI vectors will be tested in mice models of light induced retinal degeneration and laser induced choroidal neovascularization via intravitreal injections. Amount of protein produced and its biodistribution in the retina will be tested via Western blot and immunohistochemistry. Rescue of photoreceptor thinning and RPE cell death will be assessed via optical coherence tomography, fundus photography and histological analyses. Immunoreactivity of RPE with cell surface markers, CD46, CD55 and CD59 and C3 and MAC deposition will be assessed by fluorescent microscopy or western blot. Levels of iC3b will be measured by Western Blot or ELISA.

Appropriate dose for non-human primates will be determined based on mice studies. Non-human primate studies will be conducted in cynomologus monkeys via intravitreal injections. Therapeutic benefits will be evaluated based on levels of CFI proteins produced and secreted in the retina. Amount of secreted CFI protein will be measured in the retina and the choroid compared to uninjected or sham injected cohorts. Increased levels of CFI in the retina and choroid is expected to normalize complement and provide therapeutic benefits in the AMD population with rare mutations that lead to the loss or decreased amount of these protein. The non-human primate dose finding studies will enable us to establish a safe starting dose for human studies.

### Example 2: Expression of CFI in HEK Cells

An AAV2 vector comprising the CFI gene under the control of the chicken beta actin promoter (CBA) and having the nucleotide sequence of SEQ ID NO: 7 was transfected into suspension HEK293T cells in triplicate using 1 mg/L plasmid DNA. Cells were transfected with PEI at a 1:1 DNA:PEI ratio. Cells were cultured for 120 hr and sampled for analysis.

Supernatant and harvested cell samples were collected from transfected cells and exposed to either reducing (beta-mercaptoethanol) or non-reducing conditions and subjected to Western blot analysis. Western blots were probed with Quidel A313 Goat Antiserato CFI 1:1000, O/N 4° C. with rocking and then probed with Rabbit anti-Goat-HRP 1:5000, 1 h at room temperature with rocking and then visualized with chemiluminescent reagents. Robust levels of the unprocessed CFI protein (88 kDa) were observed in supernatant samples under non-reducing conditions, while very little if any CFI protein was detected in pellet samples under non-reducing conditions. High levels of unprocessed CFI were observed in the supernatant and pellet samples under reducing conditions, but processed forms of CFI (50 and 38 kDa) were also observed in the supernatant samples exposed to reducing conditions. By comparison, no detectable levels of CFI were expressed following cell transfection with a CFI-AAV3 vector.

### Example 3: Expression of CFI in Cynomolgus Monkey Eyes

Cynomolgus monkeys were dosed with AAV2 vectors having the nucleotide sequence of SEQ ID NO: 7 and containing the CBA 1.6 kb long form promoter and the CF coding sequence at 1.14e12 vg/eye in 100 μl dosing volume. After 30 days, eye samples were collected and subjected to further analysis.

Immunochemistry was performed on eye samples to detect the presence of CFI protein. Expression was observed throughout the retina. Widespread staining of ganglion cells in the ganglion layer was detected.

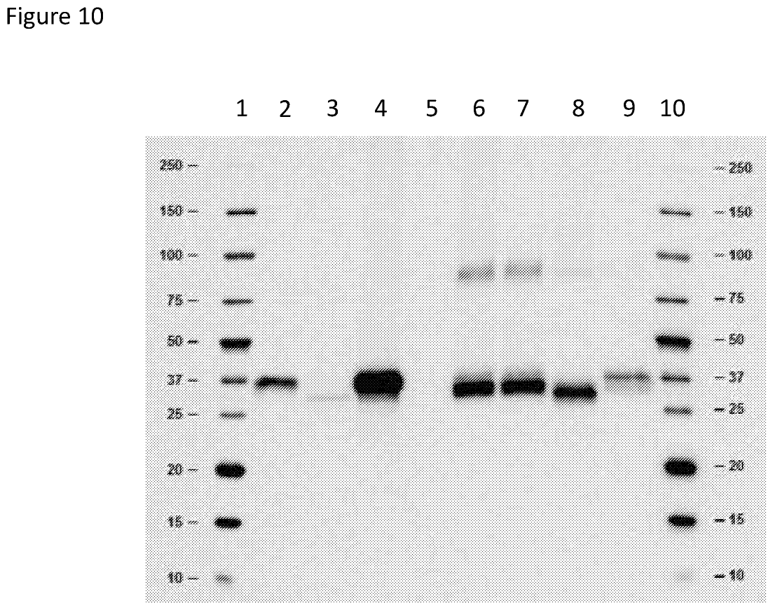

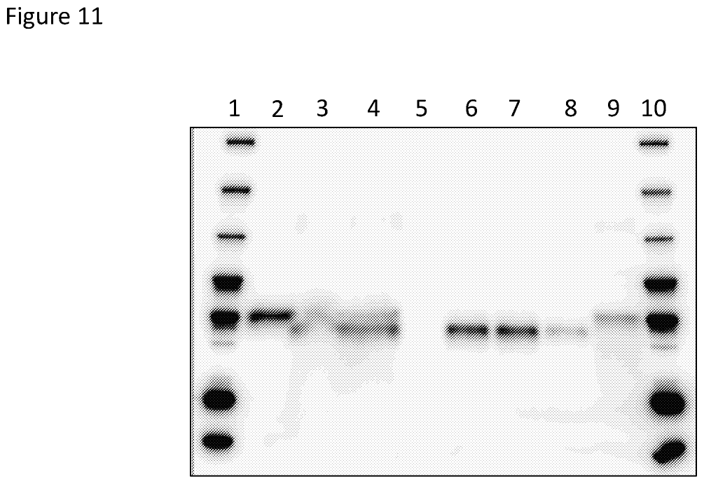

Eye samples were subjected to Western Blot analysis using reducing conditions and CFI levels were detected using a mouse anti-human CFI protein (7C9) and a secondary antibody Novus NBP-46264. Robust levels of human CFI protein were detected in the vitreous humor and in the RPE-macular region from eyes of treated animals. Western Blot levels from vitreous humor experiments are shown in FIG. 10, and Western Blot levels from RPE/choroid experiments are shown in FIG. 11.

### Example 4: CFI Cofactor Assay

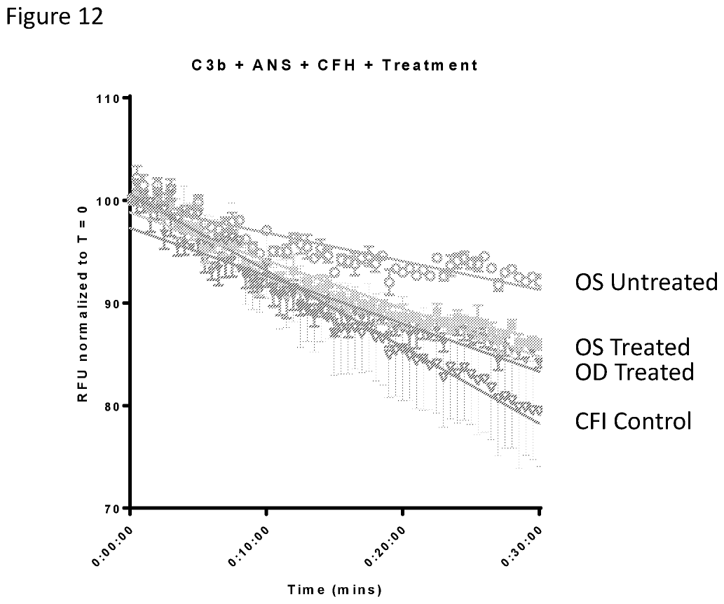

Ten micro-liters of vitreous samples taken from cynomolgus monkeys treated as described in Example 3 above diluted in PBS were mixed with C3b (Comptech cat. A114), the fluorometric substrate ANS, and CFH (Comptech cat. A137) to yield a final concentration of 0.4 mg/ml C3b, 100 μM ANS, 0.0005-0.06 mg/ml CFH and vitreous humor (diluted either at 1:10 or 1:100). As a positive control, C3b, ANS, CFH were mixed at the same concentrations as above, but with purified CFI (Comptech cat. A138). Samples were read on a SpectraMax m3 from Molecular Devices every 30 seconds for 30 minutes at 30° C. on fluorescent kinetic mode with excitation 386 nm and emission 472 nm. Results are shown in FIG. 12. The data reveals that additional CFI appears to be present in the vitreous of treated animals compared to control treated animals.

### Example 5: Distribution of CFI

The RNAscope® Assay is an advanced RNA in situ hybridization (ISH) approach with a unique RNA probe design strategy that allows simultaneous signal amplification and background suppression to achieve single-molecule visualization while preserving tissue morphology. To evaluate the pattern of AAV vector CBA promoter, GFP, and codon-optimized CFI transgene RNA in the AAV injected non-human primate (NHP) eye samples as described in Example 3 above, RNAscope® 2.5 LS Duplex ISH was performed on automation platform using the RNAscope® 2.5 LS Duplex Reagent Kit (Advanced Cell Diagnostics, Inc., Newark, Calif.). For each sample, marker expression was assessed in the optic nerve, macula, peripheral region, and ciliary bodies. Briefly, 5 μm formalin fixed, paraffin embedded (FFPE) tissue sections were pretreated with heat and protease prior to hybridization with the target oligo probes. The probes used were: Hs-CFI-O1 (ACD Cat. No. 537328), V-CBpromoter-C2 (ACD Cat. No. 423748-C2) and positive control probes Mfa-PPIB-C1/Mfa-POLR2A-C2 (ACD custom reagent) and dapB-C1/dapB-C2 (ACD Cat. No. 320758). Preamplifier, amplifier and HRP/AP-labeled oligos were then hybridized sequentially, followed by chromogenic precipitate development. Each sample was quality controlled for RNA integrity with a RNAscope® probe specific to PPIB and POLR2A RNA and for background with a probe specific to bacterial dapB RNA. Specific RNA staining signal was identified as green, punctate dots or red, punctate dots. Samples were counterstained with Gill's Hematoxylin. Images were then acquired using an Aperio AT2 digital slide scanner equipped with a 40× objective. Strong staining for both the promoter and for the CFI coding sequence were detected in the optic nerve, macula and ciliary bodies indicating the presence of the transduced AAV in those tissues.

### Example 6: Expression of CFI in Cynomolgus Monkey Eyes

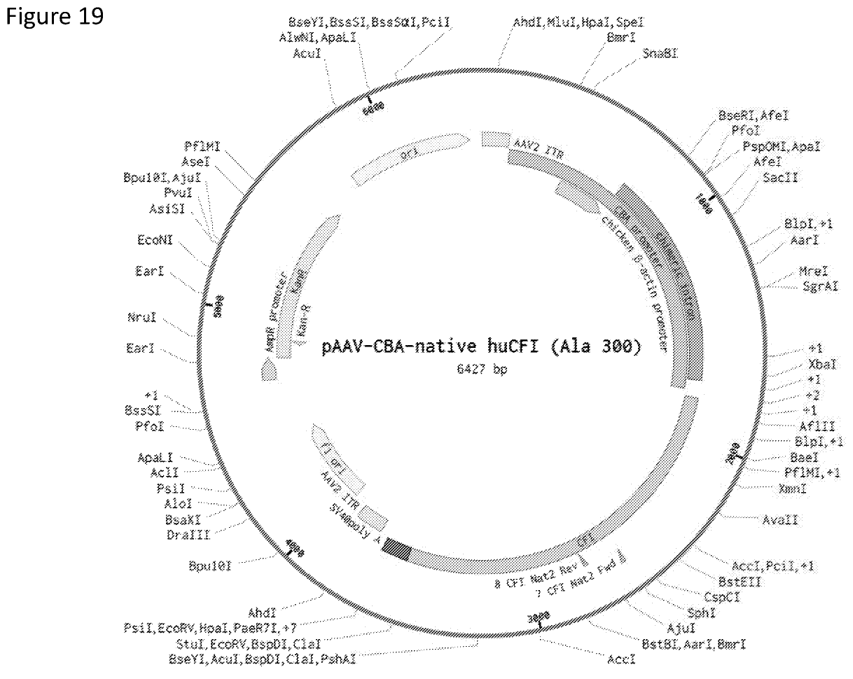

Cynomolgus monkeys were dosed intravitreally on day 1 with 100 μL of AAV2-GP2031 (see. SEQ ID NO: 33 and FIG. 19) at 5e+11 vg/eye or with 100 μL of vehicle. Animals were sacrificed and vitreous humor was collected from the eyes (both left eye and right eye) on study day 29.

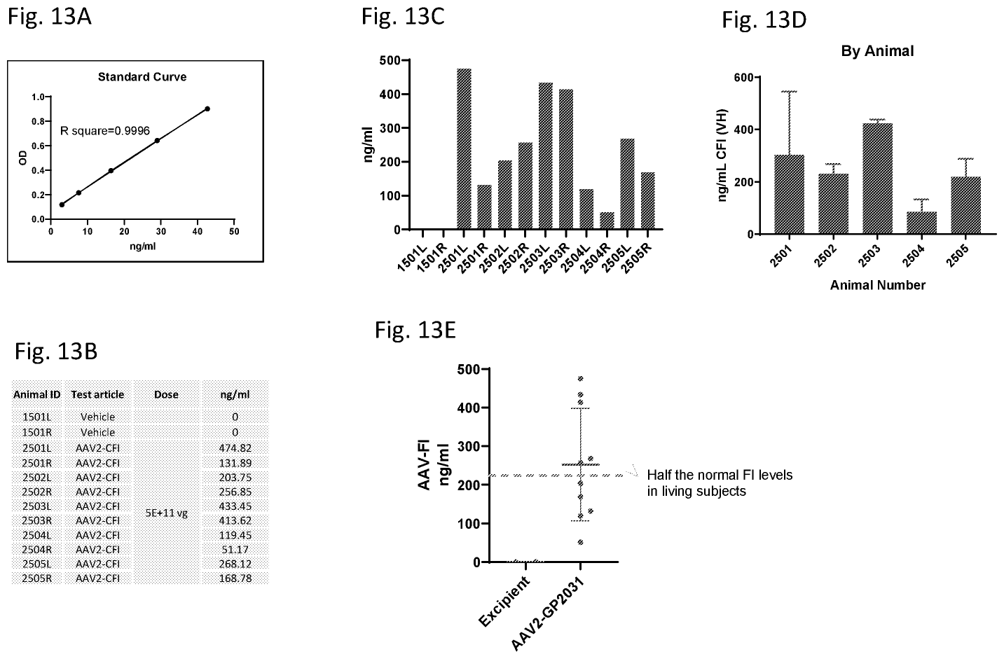

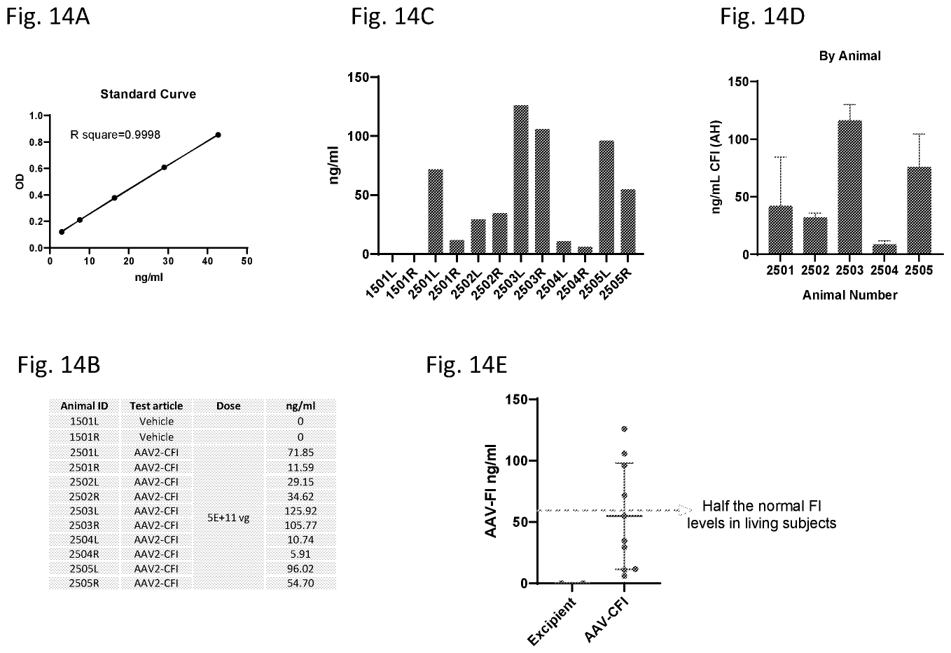

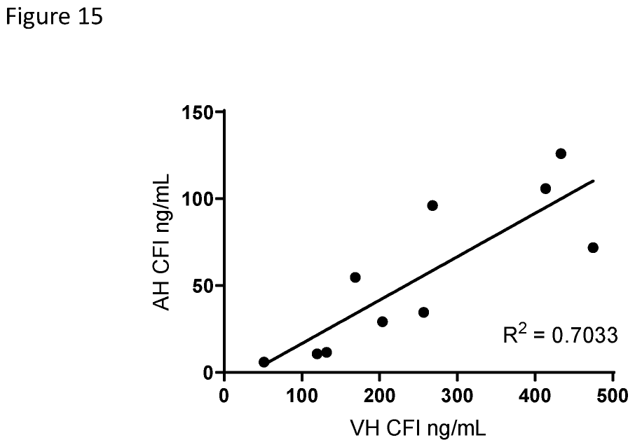

Factor I (FI) ELISA was performed using the human specific FI Microvue kit (A041, Quidel Corporation) as per the manufacturer's instructions. Vitreous humor, aqueous humor and protein extracted from eye tissue samples were diluted in sample diluent buffer provided with the kit. F1 protein was quantified using the standard curve generated with the kit standards by linear regression using Graphpad Prism software. As shown in FIGS. 13A-13E, CFI was successfully expressed in vitreous humor of both left and right eyes of cynomolgus monkeys intravitreally administered the AAV2-GP2031 construct. Moreover, expression of CFI increased in a dose-dependent manner. Similarly, FIGS. 14A-14E show that CFI was successfully expressed in aqueous humor of both left and right eyes of cynomolgus monkeys intravitreally administered the AAV2-GP2031 construct. Moreover, expression of CFI increased in a dose-dependent manner. FIG. 15 shows the correlation between CFI levels detected at different concentrations in aqueous humor and vitreous humor samples obtained from treated animals.

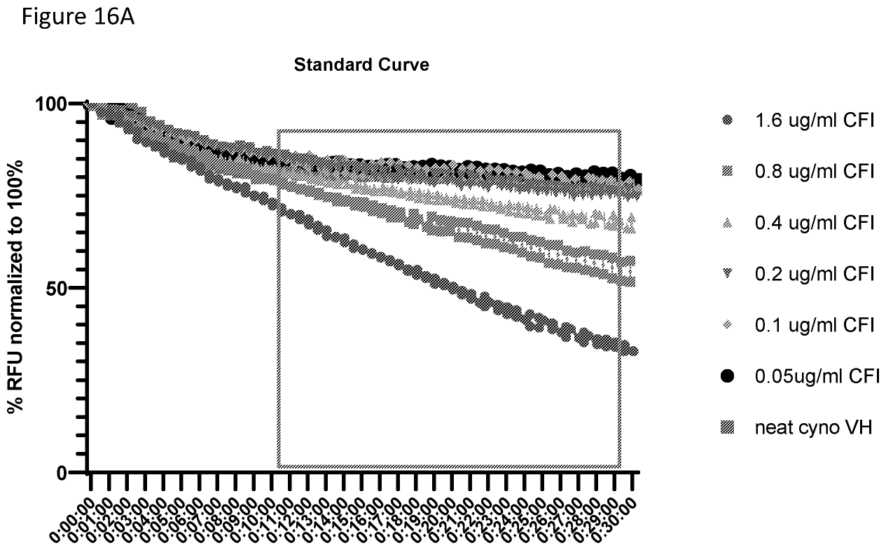

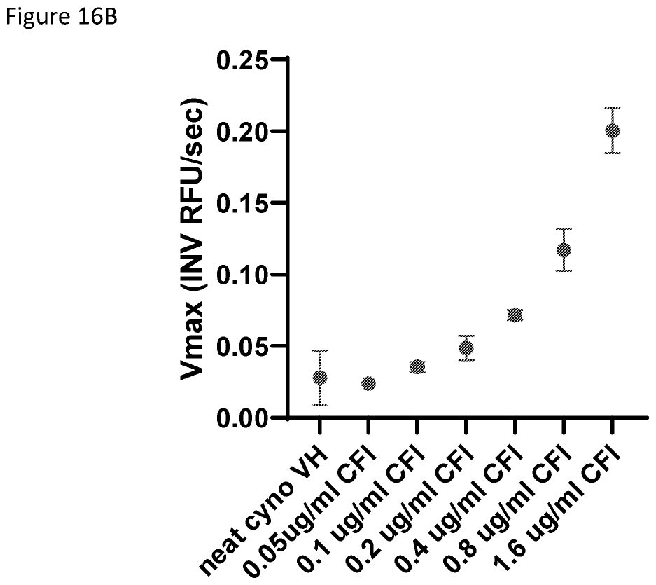

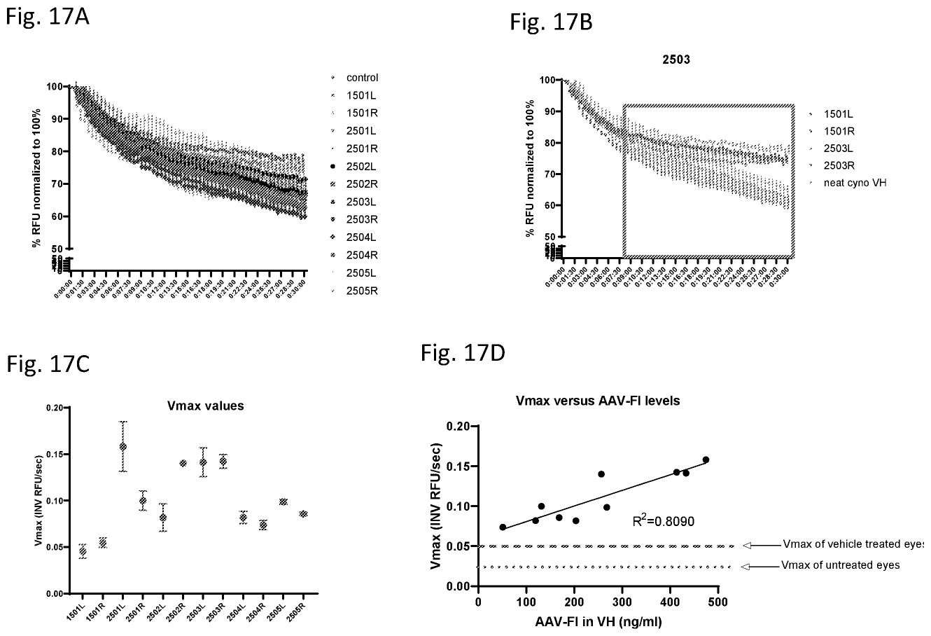

In a separate experiment, the activity of the expressed CFI protein was tested. Assay components were added to opaque half-area black polystyrene plates in the following order in a 50 μL final reaction volume: 0.02 mg purified human C3b, 5 μM ANS, I0 μL cynomolgus monkey vitreous humor and 5 μg of CFH. Reactions were mixed briefly by shaking at 4000 rpm and read over 30 minutes at 30 second intervals at 30° C. Fluorescence readings were recorded in kinetic mode with excitation set to 386 nm and emission set to 472 nm. Positive control samples included naive cynomolgus vitreous with recombinant CFI spiked in at increasing concentrations (0.05 to 1.6 ug/ml). Negative controls for reaction rate included naive cynomolgus vitreous without rCFI and samples prepared with no C3b, no CFI or no CFH. Percentage fluorescence was graphed after normalizing to time 0. As shown in FIGS. 16A and 17A-17B, CFI expressed in eyes of cynomolgus monkeys was capable of cleaving C3b in a dose-dependent manner. The kinetic plots were analyzed by assessment of the slopes. The reaction rates, i.e., the slopes of observed reduction in fluorescence at 472 nm (corresponding to C3b cleavage), were calculated for each sample, carried out in triplicate. The maximum reaction rates (Vmax) for each sample were calculated by Graphpad Prism software based on the analysis of the nonlinear regression of the kinetic activity data from 500s to 1800s. The slopes were graphed as inverse RFU/second and are shown in FIGS. 16B, 17C and 17D. As shown in FIGS. 16A-17D, CFI expressed in eyes of cynomolgus monkeys was associated with C3b cleavage in a dose-dependent manner.

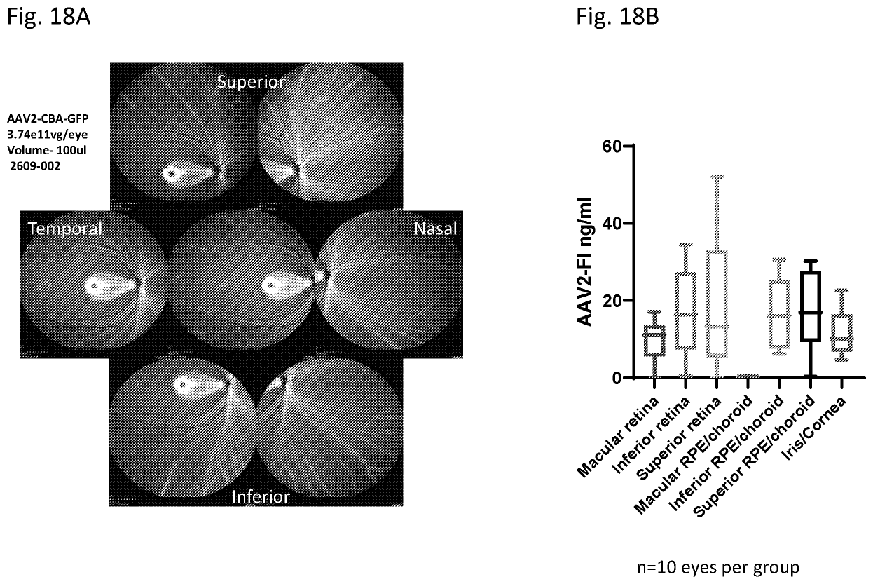

FIG. 18A is a fundus autofluorescence image of a cynomolgus eye one month post injection of AAV2-CBA-GFP and shows the biodistribution of AAV2 by GFP fluorescence. The dose injected was 3.74e+11vg and volume injected was 100 μl. FIG. 18B shows the quantification of CFI protein from tissue punches taken from different areas (macula, inferior and superior) and tissue layers of the eye. These ocular tissues (from 10 eyes) were isolated one month post-injection with AAV2-CBA-CFI at a dose of 5e+11vg in a volume of 100 μl. CFI protein was quantified using the standard curve generated using a human specific FI Microvue kit (A041, Quidel Corporation).

### Example 7: CFI Mutant Analysis

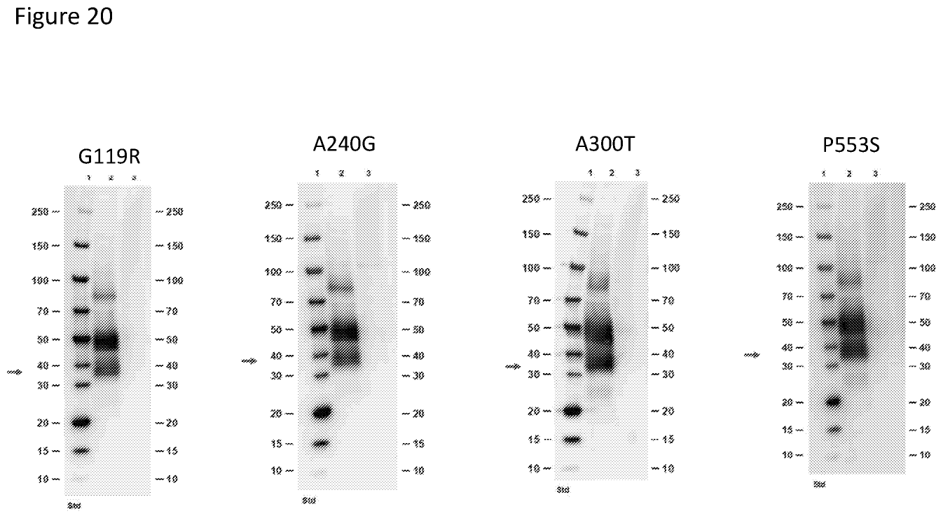

Several known, but previously uncharacterized, CFI mutant variants were produced and characterized in a functional assay. Specifically, G119R, A240G, P553S, and A300T variants were expressed in cells that were co-transfected with a gene encoding furin, and the expressed CFI protein was purified using an affinity column. As shown in FIG. 20, mature mutant CFI was produced.

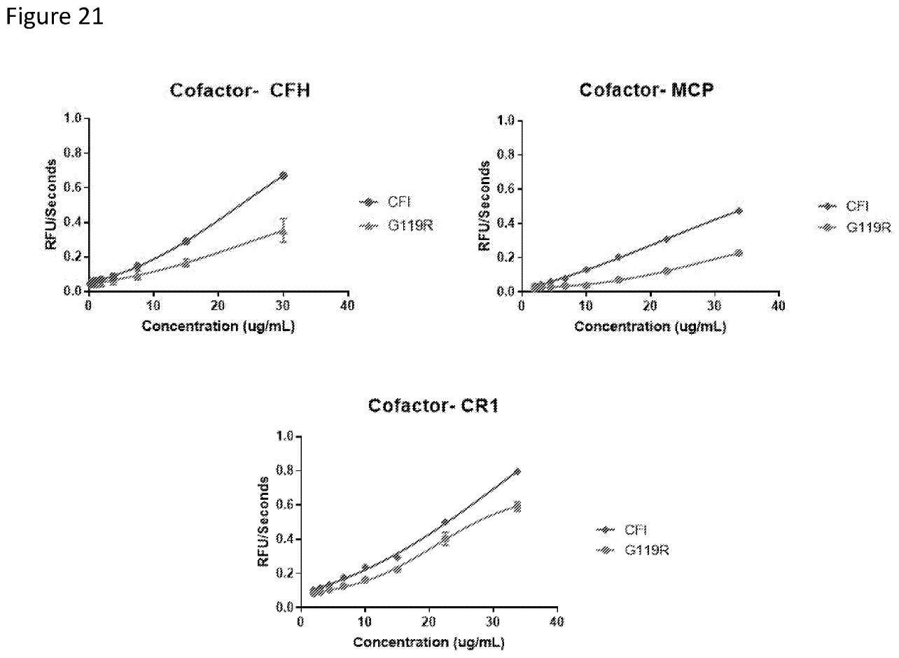

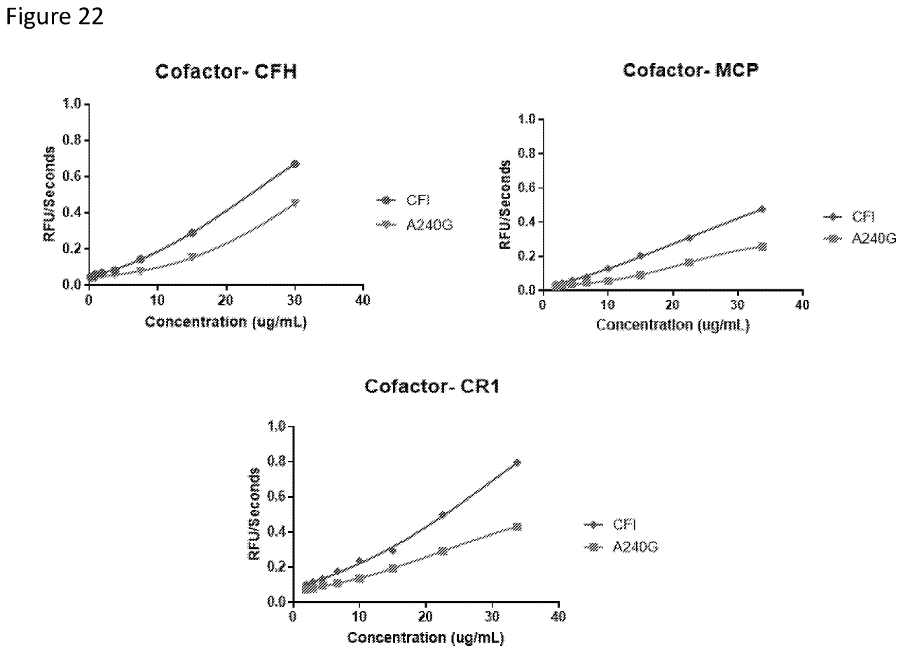

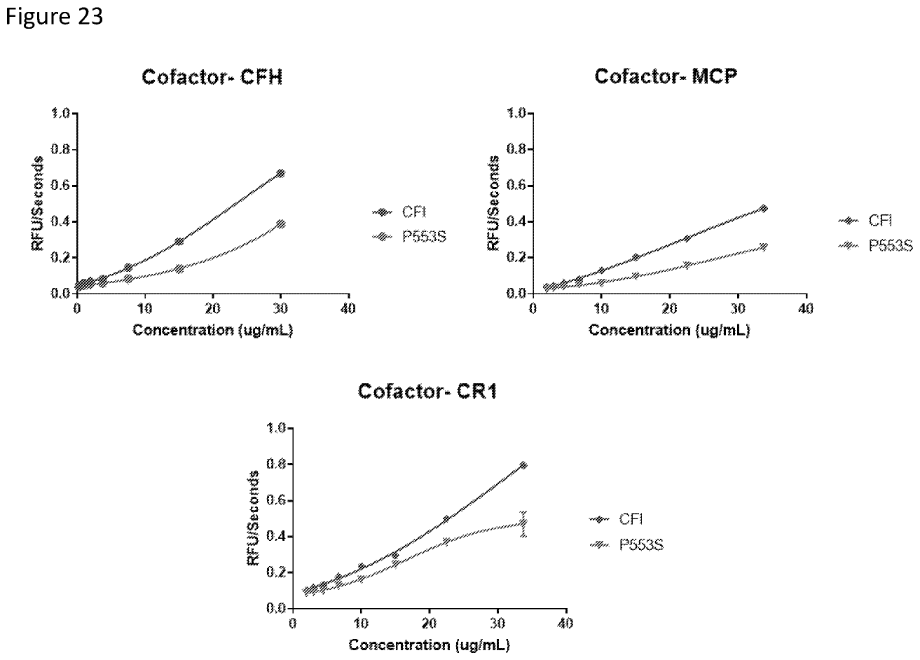

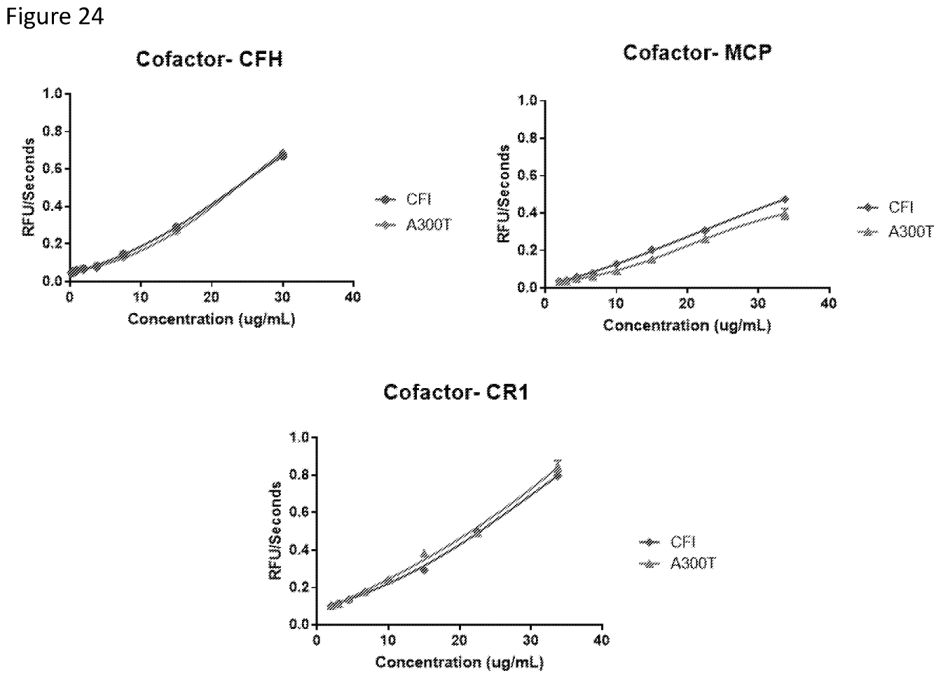

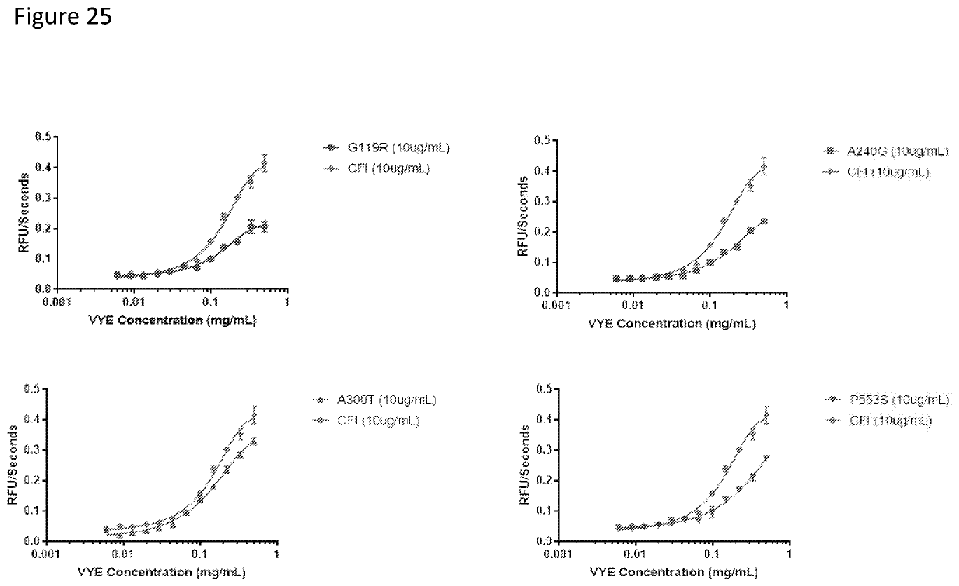

While the G119R, A24G, P553S and A300T mutants had previously been detected in AMD patients, these mutations are only a few of many, many CFI mutations that have been identified in AMD patients. Moreover, it is unclear whether any of these mutations have any impact on CFI function. We speculated that G119R, A240G, P553S, and A300T mutant proteins may be associated with reduced CFI activity. Activity of the G119R, A240G, P553S, and A300T mutant CFI proteins was tested in a fluorescence cofactor assay. Briefly, C3b was labeled with ANS, which provides a fluorescent signal. The ANS-labeled C3b was then mixed with one of three different cofactors: CFH, CR1 or MCP. These cofactors bind to CFH-, CR1- or MCP-binding domains of C3b. Increasing concentrations CFI variants (G119R, A240G, P553S, or A300T) or wildtype CFI was then added to each cofactor/ANS-C3b mixture to initiate cleavage of C3b to iC3b. Cleavage of C3b was reflected by the change in relative fluorescent units (RFUs) over time. Results from the fluorescence cofactor assays are shown in FIGS. 21-24. In a separate experiment, increasing concentrations of CFH were added to a mixture of a fixed concentration of ANS-C3b and wildtype CFI or CF variant (G119R, A240G, P553S, and A300T). Results from this experiment are shown in FIG. 25. Surprisingly, it was found that certain CFI mutations (G119R, A240G, and P553S) were associated with greatly reduced CFI protein function (FIGS. 21-23). By comparison, the A300T mutation appeared to have relatively little impact on CFI function (FIG. 24). These data suggest that patients (e.g., an AMD patient) harboring mutations (e.g., G119R, A240G, or P553S) that reduce CFI activity may be more amenable to treatment with any of the vectors disclosed herein than, for example, a patient (e.g., an AMD patient) lacking these mutations or a patient having a CFI mutation that does not have a significant impact on CFI activity (e.g., A300T).

CFI Activity Assay Protocol

Concentrated stock of ANS (ARCOS Organics #401210051) was prepared by weighing out ANS into 1 ml DMSO in a glass amber vial. 1 mL of ANS working stock (500 uM) was prepared by diluting 0.5 ul of concentrated stock with 1×TBS in a polypropylene “Eppendorf” type tube and stored at room temperature until use. 1 mL of dilute CFH (Complement Technology, Inc. Cat #A137) was prepared for each 96 well plate. CompTech plasma derived CFH material was diluted 1:5 in 1×TBS from 1.0 mg/ml to 0.2 mg/ml. The materials were then stored in an ice water bath until use. CFI standard curve samples were prepared in 1×TBS in duplicate. The test/unknown samples were diluted as appropriate in 1×TBS and then stored in ice water bath until use. The standard controls included “no C3b”, “no CFI” and “no CFH”. The plate reader was warmed to 30° C., and the 96 well plate was placed on ice or cold pack. 20 ul of C3b (Complement Technology, Cat #A114) was then plated at 1 mg/ml per well (except no C3b control wells), and 10 ul of ANS working stock per well. 10 ul of CFH was added to appropriate wells. The well contents were mixed briefly (less than 1 min) on a plate shaker at 4000 rpm. The plate was placed in a plate reader to warm to 30° C. The plate was removed and 10 ul of CFI standands and samples were added per well (except no CFI was added for control wells). The plate was read for 30 minutes every fifteen seconds at 30° C. on a SpectraMax M3 plate reader in kinetic mode with excitation set at 386 nm and emission set at 472 nm. Reactions were stopped by adding reducing Laemmli buffer and run on a gel to visualize C3b cleavage using Coomasie stain. The slope of kinetic reaction (measured between 300 and 900 seconds) was plotted versus concentration of FI standard curve and unknowns were interpolated.

### Example 8: Treatment of Patients with AMD with AAV Vectors

This study will evaluate the efficacy of the vectors of Example 1 for treating patients with AMD. Patients with AMD will be treated with any of the CFI AAV2 vectors, or a control. The vectors will be administered at varying doses between 2.5×10⁸ vg to 1.4×10¹¹ vg/per eye in about 100 μl. The vectors will be administered by intravitreal injection in a solution of PBS with additional NaCl and pluronic. Patients will be monitored for improvements in AMD symptoms.

It is expected that the CF AAV2 vector treatments will improve the AMD symptoms.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the disclosure should be determined by reference to the claims, along with their full scope of equivalents, and the specification, along with such variations.

SEQUENCE LISTING

SEQ ID NO: 1-Codon Optimized Human Complement Factor I + Kozak Sequence
GTCCAGGCGGCCGCCACCATGAAGCTTCTTCATGTTTTCCTGTTATTTCTGTGCTT
CCACTTAAGGTTTTGCAAGGTCACTTATACATCTCAAGAGGATCTGGTGGAGAAA
AAGTGCTTAGCAAAAAAATATACTCACCTCTCCTGCGATAAAGTCTTCTGCCAGC
CATGGCAGAGATGCATTGAGGGCACCTGTGTTTGTAAACTACCGTATCAGTGCCC
AAAGAATGGCACTGCAGTGTGTGCAACTAACAGGAGAAGCTTCCCAACATACTG
TCAACAAAAGAGTTTGGAATGTCTTCATCCAGGGACAAAGTTTTTAAATAACGGA
ACATGCACAGCCGAAGGAAAGTTTAGTGTTTCCTTGAAGCATGGAAATACAGAT
TCAGAGGGAATAGTTGAAGTAAAACTTGTGGACCAAGATAAGACAATGTTCATA
TGCAAAAGCAGCTGGAGCATGAGGGAAGCCAACGTGGCCTGCCTTGACCTTGGG
TTTCAACAAGGTGCTGATACTCAAAGAAGGTTTAAGTTGTCTGATCTCTCTATAA
ATTCCACTGAATGTCTACATGTCCATTGCCGAGGATTAGAGACCAGTTTGGCTGA
ATGTACTTTTACTAAGAGAAGAACTATGGGTTACCAGGATTTCGCTGATGTGGTT
TGTTATACACAGAAAGCAGATTCTCCAATGGATGACTTCTTTCAGTGTGTGAATG
GGAAATACATTTCTCAGATGAAAGCCTGTGATGGTATCAATGATTGTGGAGACCA
AAGTGATGAACTGTGTTGTAAAGCATGCCAAGGCAAAGGCTTCCATTGCAAATC
GGGTGTTTGCATTCCAAGCCAGTATCAATGCAATGGTGAGGTGGACTGCATTACA
GGGGAAGATGAAGTTGGCTGTGCAGCAGCTAGACATCCTACAATTCAAGGCTTT
GCATCTGTGGCTCAAGAAGAAACAGAAATTTTGACTGCTGACATGGATGCAGAA
AGAAGACGGATAAAATCATTATTACCTAAACTATCTTGTGGAGTTAAAAACAGA
ATGCACATTCGAAGGAAACGAATTGTGGGAGGAAAGCGAGCACAACTGGGAGA
CCTCCCATGGCAGGTGGCAATTAAGGATGCCAGTGGAATCACCTGTGGGGGAAT
TTATATTGGTGGCTGTTGGATTCTGACTGCTGCACATTGTCTCAGAGCCAGTAAA
ACTCATCGTTACCAAATATGGACAACAGTAGTAGACTGGATACACCCCGACCTTA
AACGTATAGTAATTGAATACGTGGATAGAATTATTTTCCATGAAAACTACAATGC
AGGCACTTACCAAAATGACATCGCTTTGATTGAAATGAAAAAAGACGGAAACAA
AAAAGATTGTGAGCTGCCTCGTTCCATCCCTGCCTGTGTCCCCTGGTCTCCTTACC
TATTCCAACCTAATGATACATGCATCGTTTCTGGCTGGGGACGAGAAAAAGATA
ACGAAAGAGTCTTTTCACTTCAGTGGGGTGAAGTTAAACTAATAAGCAACTGCTC
TAAGTTTTACGGAAATCGTTTCTATGAAAAAGAAATGGAATGTGCAGGTACATAT
GATGGTTCCATCGATGCCTGTAAAGGGGACTCTGGAGGCCCCTTAGTCTGTATGG
ATGCCAACAATGTGACTTATGTCTGGGGTGTTGTGAGTTGGGGGGAAAACTGTGG
ATGCCAACAATGTGACTTATGTCTGGGGTGTTGTGAGTTGGGGGGAAAACTGTGG
AAAACCAGAGTTCCCAGGTGTTTACACCAAAGTGGCCAATTATTTTGACTGGATT
AGCTACCATGTAGGAAGGCCTTTTATTTCTCAGTACAATGTATAATAAGCTTGGA
TCCAGATCTAATCAACCTC
SEQ ID NO: 2-Codon Optimized Human Complement Factor I
GCGGCCGCCACCATGAAACTGCTGCATGTCTTTCTGCTGTTTCTGTGCTTCCATCT
GCGCTCTGCAAGGTCACTTACACTTCTCAGGAGGATCTGGTCGAGAAGAAGTGT
CTGGCCAAGAAGTACACACACCTGAGCTGCGACAAGGTGTTCTGTCACCCTTGG
CAGAGATGCATCGAGGGCACCTGCGTGTGCAAGCTGCCTTACCAGTGCCCAAAG
AACGGAACCGCCGTGTGCGCAACAAATCGGCGGAGCTTTCCAACATATTGCCAG
CAGAAGAGCCTGGAGTGTCTGCACCCCGGCACCAAGTTCCTGAACAATGGCACC
TGCACAGCCGAGGGCAAGTTTTCTGTGAGCCTGAAGCACGGCAACACAGATAGC
GAGGGCATCGTGGAGGTGAAGCTGGTGGACCAGGATAAGACCATGTTCATCTGT
AAGAGCTCCTGGTCCATGAGGGAGGCAAACGTGGCATGCCTGGATCTGGGATTC
CAGCAGGGAGCAGACACACAGAGGCGCTTTAAGCTGTCCGACCTGTCTATCAAT
AGCACCGAGTGCCTGCACGTGCACTGTAGGGGCCTGGAGACATCCCTGGCAGAG
TGCACCTTCACAAAGCGGAGAACCATGGGCTACCAGGACTTTGCCGACGTGGTG
TGCTATACCCAGAAGGCCGATAGCCCAATGGACGATTTCTTTCAGTGCGTGAACG
GCAAGTATATCTCCCAGATGAAGGCCTGCGACGGCATCAATGACTGTGGCGATC
AGTCTGACGAGCTGTGCTGTAAGGCCTGTCAGGGCAAGGGCTTCCACTGCAAGA
GCGGCGTGTGCATCCCTTCCCAGTACCAGTGCAACGGCGAGGTGGATTGTATCAC
AGGAGAGGACGAAGTGGGATGCGCTGCCGCCAGACACCCAACCATCCAGGGCTT
TGCCTCTGTGGCCCAGGAGGAGACAGAGATCCTGACAGCCGACATGGATGCCGA
GAGGCGCCGGATCAAGTCTCTGCTGCCCAAGCTGAGCTGCGGCGTGAAGAATAG
GATGCACATCAGAAGGAAGCGCATCGTGGGAGGCAAGAGGGCACAGCTGGGCG
ATCTGCCTTGGCAGGTGGCCATCAAGGACGCCTCTGGCATCACCTGCGGCGGCAT
CTACATCGGAGGATGTTGGATCCTGACCGCAGCACACTGCCTGAGAGCAAGCAA
GACACACAGGTATCAGATTTGGACCACAGTGTGGATTGGATCCACCCAGACCT
GAAGAGAATCGTGATCGAGTACGTGGATAGGATCATCTTCCACGAGAACTACAA
TGCCGGCACATATCAGAACGACATCGCCCTGATCGAGATGAAGAAGGATGGCAA
TAAGAAGGACTGTGAGCTGCCACGCTCCATCCCTGCATGCGTGCCCTGGAGCCCC
TATCTGTTCCAGCCCAACGATACCTGTATCGTGTCCGGCTGGGGCCGCGAGAAGG
ACAATGAGCGGGTGTTTTCTCTGCAGTGGGGCGAGGTGAAGCTGATCTCCAACTG
TTCTAAGTTCTACGGCAATCGGTTTTATGAGAAGGAGATGGAGTGCGCCGGCACC
TACGATGGCAGCATCGACGCCTGTAAGGGCGATTCCGGAGGACCACTGGTGTGC
ATGGACGCAAACAATGTGACATACGTGTGGGGCGTGGTGTCCTGGGGCGAGAAT
TGCGGCAAGCCAGAGTTTCCCGGCGTGTATACCAAGGTGGCCAACTATTTTGATT
GGATTTCCTACCATGTCGGGAGACCATTCATTTCACAGTATAACGTGTAATAAGC
TTGGATCCAGATCT
SEQ ID NO: 3-Non-Codon Optimized Human Complement Factor I
GCGGCCGCCACCATGAAGCTTCTTCATGTTTTCCTGTTATTTCTGTGCTTCCACTT
AAGGTTTTGCAAGGTCACTTATACATCTCAAGAGGATCTGGTGGAGAAAAAGTG
CTTAGCAAAAAAATATACTCACCTCTCCTGCGATAAAGTCTTCTGCCAGCCATGG
CAGAGATGCATTGAGGGCACCTGTGTTTGTAAACTACCGTATCAGTGCCCAAAG
AATGGCACTGCAGTGTGTGCAACTAACAGGAGAAGCTTCCCAACATACTGTCAA
CAAAAGAGTTTGGAATGTCTCCATCCAGGGACAAAGTTTTTAAATAACGGAACAT
CCACAGCCGAAGGAAAGTTTAGTGTTTCCTTGAAGCATGGAAATACAGATTCAG
AGGGAATAGTTGAAGTAAAACTTGTGGACCAAGATAAGACAATGTTCATATGCA
AAAGCAGCTGGAGCATGAGGGAAGCCAACGTGGCCTGCCTTGACCTTGGGTTTC
AACAAGGTGCTGATACTCAAAGAAGGTTTAAGTTGTCTGATCTCTCTATAAATTC
CACTGAATGTCTACATCTGCATTGCCGAGGATTAGAGACCAGTTTGGCTGAATGT
ACTTTTACTAAGAGAAGAACTATGGGTTACCAGGATTTCGCTGATGTGGTTTGTT
ATACACAGAAAGCAGATTCTCCAATGGATGACTTCTTTCAGTGTGTGAATGGGAA
ATACATTTCTCAGATGAAAGCCTGTGATGGTATCAATGATTGTGGAGACCAAAGT
GATGAACTGTGTTGTAAAGCATGCCAAGGCAAAGGCTTCCATTGCAAATCGGGT
GTTTGCATTCCAAGCCAGTATCAATGCAATGGTGAGGTGGACTGCATTACAGGG
GAAGATGAAGTTGGCTGTGCAGCAGCTAGACATCCTACAATTCAAGGCTTTGCAT
CTGTGGCTCAAGAAGAAACAGAAATTTTGACTGCTGACATGGATGCAGAAAGAA
GACGGATAAAATCATTATTACCTAAACTATCTTGTGGAGTTAAAAACAGAATGCA
CATTCGAAGGAAACGAATTGTGGGAGGAAAGCGAGCACAACTGGGAGACCTCCC
ATGGCAGGTGGCAATTAAGGATGCCAGTGGAATCACCTGTGGGGGAATTTATAT
TGGTGGCTGTTGGATTCTGACTGCTGCACATTGTCTCAGAGCCAGTAAAACTCAT
CGTTACCAAATATGGACAACAGTAGTAGACTGGATACACCCCGACCTTAAACGT
ATAGTAATTGAATACGTGGATAGAATTATTTTCCATGAAAACTACAATGCAGGCA
CTTACCAAAATGACATCGCTTTGATTGAAATGAAAAAAGACGGAAACAAAAAAG
ATTGTGAGCTGCCTCGTTCCATCCCTGCCTGTGTCCCCTGGTCTCCTTACCTATTC
CAACCTAATGATACATGCATCGTTTCTGGCTGGGGACGAGAAAAAGATAACGAA
AGAGTCTTTTCACTTCAGTGGGGTGAAGTTAAACTAATAAGCAACTGCTCTAAGT
TTTACGGAAATCGTTTCTATGAAAAAGAAATGGAATGTGCAGGTACATATGATG
GTTCCATCGATGCCTGTAAAGGGGACTCTGGAGGCCCCTTAGTCTGTATGGATGC
CAACAATGTGACTTATGTCTGGGGTGTTGTGAGTTGGGGGGAAAACTGTGGAAA
ACCAGAGTTCCCAGGTGTTTACACCAAAGTGGCCAATTATTTTGACTGGATTAGC
TACCATGTAGGAAGGCCTTTTATTTCTCAGTACAATGTATAATAAGCTTGGATCC
AGATCT
SEQ ID NO: 4: SFTL Sequence
SFTL
SEQ ID NO: 5: CFI Nucleotide Sequence
ATGAAGCTTCTTCATGTTTTCCTGTTATTTCTGTGCTTCCACTTAAGGTTTTGCAA
GGTCACTTATACATCTCAAGAGGATCTGGTGGAGAAAAAGTGCTTAGCAAAAAA
ATATACTCACCTCTCCTGCGATAAAGTCTTCTGCCAGCCATGGCAGAGATGCATT
GAGGGCACCTGTGTTTGTAAACTACCGTATCAGTGCCCAAAGAATGGCACTGCA
GTGTGTGCAACTAACAGGAGAAGCTTCCCAACATACTGTCAACAAAAGAGTTTG
GAATGTCTTCATCCAGGGACAAAGTTTTTAAATAACGGAACATGCACAGCCGAA
GGAAAGTTTAGTGTTTCCTTGAAGCATGGAAATACAGATTCAGAGGGAATAGTT
GAAGTAAAACTTGTGGACCAAGATAAGACAATGTTCATATGCAAAAGCAGCTGG
AGCATGAGGGAAGCCAACGTGGCCTGCCTTGACCTTGGGTTTCAACAAGGTGCT
GATACTCAAAGAAGGTTTAAGTTGTCTGATCTCTCTATAAATTCCACTGAATGTC
TACATGTGCATTGCCGAGGATTAGAGACCAGTTTGGCTGAATGTACTTTTACTAA
GAGAAGAACTATGGGTTACCAGGATTTCGCTGATGTGGTTTGTTATACACAGAAA
GCAGATTCTCCAATGGATGACTTCTTTCAGTGTGTGAATGGGAAATACATTTCTC
AGATGAAAGCCTGTGATGGTATCAATGATTGTGGAGACCAAAGTGATGAACTGT
GTTGTAAAGCATGCCAAGGCAAAGGCTTCCATTGCAAATCGGGTGTTTGCATTCC
AAGCCAGTATCAATGCAATGGTGAGGTGGACTGCATTACAGGGGAAGATGAAGT
TGGCTGTGCAGGCTTTGCATCTGTGACTCAAGAAGAAACAGAAATTTTGACTGCT
GACATGGATGCAGAAAGAAGACGGATAAAATCATTATTACCTAAACTATCTTGT
GGAGTTAAAAACAGAATGCACATTCGAAGGAAACGAATTGTGGGAGGAAAGCG
AGCACAACTGGGAGACCTCCCATGGCAGGTGGCAATTAAGGATGCCAGTGGAAT
CACCTGTGGGGGAATTTATATTGGTGGCTGTTGGATTCTGACTGCTGCACATTGT
CTCAGAGCCAGTAAAACTCATCGTTACCAAATATGGACAACAGTAGTAGACTGG
ATACACCCCGACCTTAAACGTATAGTAATTGAATACGTGGATAGAATTATTTTCC
ATGAAAACTACAATGCAGGCACTTACCAAAATGACATCGCTTTGATTGAAATGA
AAAAAGACGGAAACAAAAAAGATTGTGAGCTGCCTCGTTCCATCCCTGCCTGTG
TCCCCTGGTCTCCTTACCTATTCCAACCTAATGATACATGCATCGTTTCTGGCTGG
GGACGAGAAAAAGATAACGAAAGAGTCTTTTCACTTCAGTGGGGTGAAGTTAAA
CTAATAAGCAACTGCTCTAAGTTTTACGGAAATCGTTTCTATGAAAAAGAAATGG
AATGTGCAGGTACATATGATGGTTCCATCGATGCCTGTAAAGGGGACTCTGGAG
GCCCCTTAGTCTGTATGGATGCCAACAATGTGACTTATGTCTGGGGTGTTGTGAG
TTGGGGGGAAAACTGTGGAAAACCAGAGTTCCCAGGTGTTTACACCAAAGTGGC
CAATTATTTTGACTGGATTAGCTACCATGTAGGAAGGCCTTTTATTTCTCAGTACA
ATGTATAA
SEQ ID NO: 6-1.6 KB CBA Promoter
ACGCGTGITAACTAGTGGCCCGCCTGGCTGACCGCCCAACGACCCCCGCCCATTG
ACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTA
TCATATGCCAAGTACGCCCCCTATTGACGTCAATGACGGTAAATGGCCCGCCTGG
CATTATGCCCAGTACATGACCTTATGGGACTTTCCTACTTGGCAGTACATCTACG
TATTAGTCATCGCTATTACCATGGTCGAGGTGAGCCCCACGTTCTGCTTCACTCTC
CCCATCTCCCCCCCCTCCCCACCCCCAATTTTGTATTTATTTATTTTTTAATTATTT
TGTGCAGCGATGGGGGCGGGGGGGGGGGGGGGGCGCGCGCCAGGCGGGGCGGG
GCGGGGCGAGGGGCGGGGCGGGGCGAGGCGGAGAGGTGCGGCGGCAGCCAATC
AGAGCGGCGCGGTCCGAAAGTTTCCTTTTATGGCGAGGCGGCGGCGGCGGCGGC
CCTATAAAAAGCGAAGCGCGCGGCGGGCGGGAGTCGCTGCGACGCTGCCTTCGC
CCCGTGCCCCGCTCCGCCGCCGCCTCGCGCCGCCCGCCCCGGCTCTGACTGACCG
CGTTACTCCCACAGGTGAGCGGGCGGGACGGCCCTTCTCCTCCGGGCTGTAATTA
GCGCTTGGTTTAATGACGGCTTGTTTCTTTTCTGTGGCTGCGTGAAAGCCTTGAGG
GGCTCCGGGAGGGCCCTTTGTGCGGGGGGGAGCGGCTCGGGGGGTGCGTGCGTG
TGTGTGTGCGTGGGGAGCGCCGCGTGCGGCCCGCGCTGCCCGGCGGCTGTGAGC
GCTGCGGGGGCGGCGCGGGGCTTTGTGCGCTCCGCAGTGTGCGCGAGGGGAGCG
CGGCCGGGGGCGGTGCCCCGCGGTGCGGGGGGGGCTGCGAGGGGAACAAAGGC
TGCGTGCGGGGTGTGTGCGTGGGGGGGTGAGCAGGGGGTGTGGGCGCGGCGGTC
GGGCTGTAACCCCCCCCTGCACCCCCCTCCCCGAGTTGCTGAGCACGGCCCGGCT
TCGGGTGCGGGGCTCCGTACGGGGCGTGGCGCGGGGCTCGCCGTGCCGGGCGGG
GGGTGGCGGCAGGTGGGGGTGCCGGGCGGGGCGGGGCCGCCTCGGGCCGGGGA
GGGCTCGGGGGAGGGGCGCGGCGGCCCCCGGAGCGCCGGCGGCTGTCGAGGCG
CGGCGAGCCGCAGCCATTGCCTTTTATGGTAATCGTGCGAGAGGGCGCAGGGAC
TTCCTTTGTCCCAAATCTGTGCGGAGCCGAAATCTGGGAGGCGCCGCCGCACCCC
CTCTAGCGGGCGCGGGGCGAAGCGGTGCGGCGCCGGCAGGAAGGAAATGGGCG
GGGAGGGCCTTCGTGCGTCGCCGCGCCGCCGTCCCCTTCTCCCTCTCCAGCCTCG
GGGCTGTCCGCGGGGGGACGGCTGCCTTCGGGGGGGACGGGGCAGGGCGGGGTT
CGGCTTCTGGCGTGTGACCGGCGGCTCTAGAGCCTCTGCTAACCATGTTCATGCC
TTCTTCTTTTTCCTACAGCTCCTGGGCAACGTGCTGGTTATTGTGCTGTCTCATCA
TTTTGGCAAA
SEQ ID NO: 7-Representative CFI AAV vector with CBA promoter
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTGTTAACT
AGTGGCCCGCCTGGCTGACCGCCCAACGACCCCCGCCCATTGACGTCAATAATG
ACGTATGTTCCCATAGTAACGCCAATAGGGACTTTCCATTGACGTCAATGGGTGG
ACTATTTACGGTAAACTGCCCACTTGGCAGTACATCAAGTGTATCATATGCCAAG
TACGCCCCCTATTGACGTGAATGACGGTAAATGGCCCGCCTGGCATTATGCCCAG
TACATGACCTTATGGGACTTTCCTACTTGGCAGTACATCTACGTATTAGTCATCGC
TATTACCATGGTCGAGGTGAGCCCCACGTTCTGCTTCACTCTCCCCATCTCCCCCC
CCTCCCCACCCCCAATTTTGTATTTATTTATTTTTTAATTATTTTGTGCAGCGATGG
GGGCGGGGGGGGGGGGGGGGCGCGCGCCAGGCGGGGCGGGGCGGGGCGAGGG
GCGGGGCGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGC
GAAGCGCGCGGCGGGCGGGAGTCGCTGCGACGCTGCCTTCGCCCCGTGCCCCGC
TCCGCCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGTTTA
ATGACGGCTTGTTTCTTTTCTGTGGCTGCGTGAAAGCCTTGAGGGGCTCCGGGAG
GGCCCTTTGTGCGGGGGGGAGCGGCTCGGGGGGTGGGTGCGTGTGTGTGTGCGT
GGGGAGCGCCGCGTGCGGCCCGCGCTGCCCGGCGGCTGTGAGCGCTGCGGGCGC
GGCGCGGGGCTTTGTGCGCTCCGCAGTGTGCGCGAGGGGAGCGCGGCCGGGGGC
GGTGCCCCGCGGTGCGGGGGGGGCTGCGAGGGGAACAAAGGCTGCGTGCGGGG
TGTGTGCGTGGGGGGGTGAGCAGGGGGTGTGGGCGCGGCGGTCGGGCTGTAACC
CCCCCCTGCACCCCCCTCCCCGAGTTGCTGAGCACGGCCCGGCTTCGGGTGCGGG
GCTCCGTACGGGGCGTGGCGCGGGGCTCGCCGTGCCGGGCGGGGGGTGGCGGCA
GGTGGGGGTGCCGGGCGGGGCGGGGCCGCCTCGGGCCGGGGAGGGCTCGGGGG
AGGGGCGCGGCGGCCCCCGGAGCGCCGGCGGCTGTCGAGGCGCGGCGAGCCGC
AGCCATTGCCTTTTATGGTAATCGTGCGAGAGGGCGCAGGGACTTCCTTTGTCCC
AAATCTGTGCGGAGCCGAAATCTGGGAGGCGCCGCCGCACCCCCTCTAGGGGGC
GCGGGGCGAAGCGGTGCGGCGCCGGCAGGAAGGAAATGGGCGGGGAGGGCCTT
CGTGCGTCGCCGCGCCGCCGTCCCCTTCTCCCTCTCCAGCCTCGGGGCTGTCCGC
GGGGGGACGGCTGCCTTCGGGGGGGACGGGGCAGGGCGGGGTTCGGCTTCTGGC
GTGTGACCGGCGGCTCTAGAGCCTCTGCTAACCATGTTCATGCCTTCTTCTTTTTC
CTACAGCTCCTGGGCAACGTGCTGGTTATTGTGCTGTCTCATCATTTTGGCAAAA
CCGGTCTCGAAGGCCTGCAGGCGGCCGCCGCCACCATGAAGCTTCTTCATGTTTT
CCTGTTATTTCTGTGCTTCCACTTAAGGTTTTGCAAGGTCACTTATACATCTCAAG
AGGATCTGGTGGAGAAAAAGTGCTTAGCAAAAAAATATACTCACCTCTCCTGCG
ATAAAGTCTTCTGCCAGCCATGGCAGAGATGCATTGAGGGCACCTGTGTTTGTAA
ACTACCGTATCAGTGCCCAAAGAATGGCACTGCAGTGTGTGCAACTAACAGGAG
AAGCTTCCCAACATACTGTCAACAAAAGAGTTTGGAATGTCTTCATCCAGGGACA
AAGTTTTTAAATAACGGAACATGCACAGCCGAAGGAAAGTTTAGTGTTTCCTTGA
AGCATGGAAATACAGATTCAGAGGGAATAGTTGAAGTAAAACTTGTGGACCAAG
ATAAGACAATGTTCATATGCAAAAGCAGCTGGAGCATGAGGGAAGCCAACGTGG
CCTGCCTTGACCTTGGGTTTCAACAAGGTGCTGATACTCAAAGAAGGTTTAAGTT
GTCTGATCTCTCTATAAATTCCACTGAATGTCTACATGTGCATTGCCGAGGATTA
GAGACCAGTTTGGCTGAATGTACTTTTACTAAGAGAAGAACTATGGGTTACCAGG
ATTTCGCTGATGTGGTTTGTTATACACAGAAAGCAGATTCTCCAATGGATGACTT
CTTTCAGTGTGTGAATGGGAAATACATTTCTCAGATGAAAGCCTGTGATGGTATC
AATGATTGTGGAGACCAAAGTGATGAACTGTGTTGTAAAGCATGCCAAGGCAAA
GGCTTCCATTGCAAATCGGGTGTTTGCATTCCAAGCCAGTATCAATGCAATGGTG
AGGTGGACTGCATTACAGGGGAAGATGAAGTTGGCTGTGCAGGCTTTGCATCTGT
GACTCAAGAAGAAACAGAAATTTTGACTGCTGACATGGATGCAGAAAGAAGACG
GATAAAATCATTATTACCTAAACTATCTTGTGGAGTTAAAAACAGAATGCACATT
CGAAGGAAACGAATTGTGGGAGGAAAGCGAGCACAACTGGGAGACCTCCCATG
GCAGGTGGCAATTAAGGATGCCAGTGGAATCACCTGTGGGGGAATTTATATTGG
TGGCTGTTGGATTCTGACTGCTGCACATTGTCTCAGAGCCAGTAAAACTCATCGT
TACCAAATATGGACAACAGTAGTAGACTGGATACACCCCGACCTTAAACGTATA
GTAATTGAATACGTGGATAGAATTATTTTCCATGAAAACTACAATGCAGGCACTT
ACCAAAATGACATCGCTTTGATTGAAATGAAAAAAGACGGAAACAAAAAAGATT
GTGAGCTGCCTCGTTCCATCCCTGCCTGTGTCCCCTGGTCTCCTTACCTATTCCAA
CCTAATGATACATGCATCGTTTCTGGCTGGGGACGAGAAAAAGATAACGAAAGA
GTCTTTTCACTTCAGTGGGGTGAAGTTAAACTAATAAGCAACTGCTCTAAGTTTT
ACGGAAATCGTTTCTATGAAAAAGAAATGGAATGTGCAGGTACATATGATGGTT
CCATCGATGCCTGTAAAGGGGACTCTGGAGGCCCCTTAGTCTGTATGGATGCCAA
CAATGTGACTTATGTCTGGGGTGTTGTGAGTTGGGGGGAAAACTGTGGAAAACC
AGAGTTCCCAGGTGTTTACACCAAAGTGGCCAATTATTTTGACTGGATTAGCTAC
CATGTAGGAAGGCCTTTTATTTCTCAGTACAATGTATAATAAGATATCGATACAT
TGATGAGTTTGGACAAACCACAACTAGAATGCAGTGAAAAAAATGCTTTATTTGT
GAAATTTGTGATGCTATTGCTTTATTTGTAACCATTATAAGCTGCAATAAACAAG
ATATCGTTAACTCGAGGGATCCCACGTGCTGATTTTGTAGGTAACCACGTGCGGA
CCGAGCGGCCGCAGGAACCCCTAGTTGATGGAGTTGGCCACTCCCTCTCTGCGCGC
TCGCTCGCTCACTGAGGCCGGGCGACCAAAGGTCGCCCGACGCCCGGGCTTTGC
CCGGGCGGCCTCAGTGAGCGAGCGAGCGCGCAGCTGCCTGCAGGGGCGCCTGAT
GCGGTATTTTCTCCTTACGCATCTGTGCGGTATTTCACACCGCATACGTCAAAGC
AACCATAGTACGCGCCCTGTAGCGGCGCATTAAGCGCGGCGGGTGTGGTGGTTA
CGCGCAGCGTGACCGCTACACTTGCCAGCGCCCTAGCGCCCGCTCCTTTCGCTTT
CTTCCCTTCCTTTCTCGCCACGTTCGCCGGCTTTCCCCGTCAAGCTCTAAATCGGG
GGCTCCCTTTAGGGTTCCGATTTAGTGCTTTACGGCACCTCGACCCCAAAAAACT
TGATTTGGGTGATGGTTCACGTAGTGGGCCATCGCCCTGATAGACGGTTTTTCGC
CCTTTGACGTTGGAGTCCACGTTCTTTAATAGTGGACTCTTGTTCCAAACTGGAA
CAACACTCAACCCTATCTCGGGCTATTCTTTTGATTTATAAGGGATTTTGCCGATT
TCGGCCTATTGGTTAAAAAATGAGCTGATTTAACAAAAATTTAACGCGAATTTTA
ACAAAATATTAACGTTTACAATTTTATGGTGCACTCTCAGTACAATCTGCTCTGAT
GCCGCATAGTTAAGCCAGCCCCGACACCCGCCAACACCCGCTGACGCGCCCTGA
CGGGCTTGTCTGCTCCCGGCATCCGCTTACAGACAAGCTGTGACCGTCTCCGGGA
GCTGCATGTGTCAGAGGTTTTCACCGTCATCACCGAAACGCGCGAGACGAAAGG
GCCTCGTGATACGCCTATTTTTATAGGTTAATGTCATGATAATAATGGTTTCTTAG
ACGTCAGGTGGCACTTTTCGGGGAAATGTGCGCGGAACCCCTATTTGTTTATTTTT
CTAAATACATTCAAATATGTATCCGCTCATGAGACAATAACCCTGATAAATGCTT
CAATAATATTGAAAAAGGAAGAGTATGAGTATTCAACATTTCCGTGTCGCCCTTA
TTCCCTTTTTTGCGGCATTTTGCCTTCCTGTTTTTGCTCACCCAGAAACGCTGGTG
AAAGTAAAAGATGCTGAAGATCAGTTGGGTGCACGAGTGGGTTACATCGAACTG
GATCTCAACAGCGGTAAGATCCITGAGAGTTTTCGCCCCGAAGAACGTTTTCCAA
TGATGAGCACTTTTAAAGTTCTGCTATGTGGCGCGGTATTATCCCGTATTGACGC
CGGGCAAGAGCAACTCGGTCGCCGCATACACTATTCTCAGAATGACTTGGTTGA
GTACTCACCAGTCACAGAAAAGCATCTTACGGATGGCATGACAGTAAGAGAATT
ATGCAGTGCTGCCATAACCATGAGTGATAACACTGCGGCCAACTTACTTCTGACA
ACGATCGGAGGACCGAAGGAGCTAACCGCTTTTTTGCACAACATGGGGGATCAT
GTAACTCGCCTTGATCCTTGGGAACCGGAGCTGAATGAAGCCATACCAAACGAC
GAGCGTGACACCACGATGCCTGTAGCAATGGCAACAACGTTGCGCAAACTATTA
ACTGGCGAACTACTTACTCTAGCTTCCCGGCAACAATTAATAGACTGGATGGAGG
CGGATAAAGTTGCAGGACCACTTCTGCGCTCGGCCCTTCCGGCTGGCTGGTTTAT
TGCTGATAAATCTGGAGCCGGTGAGCGTGGGTCTCGCGGTATCATTGCAGCACTG
GGGCCAGATGGTAAGCCCTCCCGTATCGTAGTTATCTACACGACGGGGAGTCAG
GCAACTATGGATGAACGAAATAGACAGATCGCTGAGATAGGTGCCTCACTGATT
AAGCATTGGTAACTGTCAGACCAAGTTTACTCATATATACTTTAGATTGATTTAA
AACTTCATTTTTAATTTAAAAGGATCTAGGTGAAGATCCTTTTTGATAATCTCATG
ACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAA
ACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCC
TTCTAGTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACACC
GAACTGAGATACCTACAGCGTGAGCTATGAGAAAGCGCCACGCTTCCCGAAGGG
AGAAAGGCGGACAGGTATCCGGTAAGCGGCAGGGTCGGAACAGGAGAGCGCAC
GAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTAT
GGAAAAACGCCAGCAACGCGGCCTTTTTACGGTTCCTGGCCTTTTGCTGGCCTTT
TGCTCACATGT
SEQ ID NO: 8 CRALBP Promoter
ACGCGTTAACTAGTACCCTGGTGGTGGTGGTGGGGGGGGGGGGGTGCTCTCTCA
GCAACCCCACCCCGGGATCTTGAGGAGAAAGAGGGCAGAGAAAAGAGGGAATG
GGACTGGCCCAGATCCCAGCCCCACAGCCGGGCTTCCACATGGCCGAGCAGGAA
CTCCAGAGCAGGAGCACACAAAGGAGGGCTTTGATGCGCCTCCAGCCAGGCCCA
GGCCTCTCCCCTCTCCCCTTTCTCTCTGGGTCTTCCTTTGCCCCACTGAGGGCCTC
CTGTGAGCCCGATTTAACGGAAACTGTGGGCGGTGAGAAGTTCCTTATGACACA
CTAATCCCAACCTGCTGACCGGACCACGCCTCCAGCGGAGGGAACCTCTAGAGC
TCCAGGACATTCAGGTACCAGGTAGCCCCAAGGAGGAGCTGCCGACCATCGAT
SEQ ID NO: 9 EF1a Promoter
ACGCGTTAACTAGTGGGCAGAGCGCACATCGCCCACAGTCCCCGAGAAGTTGGG
GGGAGGGGTCGGCAATTGAACCGGTGCCTAGAGAAGGTGGCGCGGGGTAAACT
GGGAAAGTGATGTCGTGTACTGGCTCCGCCTTTTTCCCGAGGGTGGGGGAGAAC
CGTATATAAGTGCAGTAGTCGCCGTGAACGTTCTTTTTCGCAACGGGTTTGCCGC
CAGAACACAG
SEQ ID NO: 10-SV40i Intron
GTAAGTTTAGTCTTTTTGTCTTTTATTTCAGGTCCCGGATCCGGTGGTGGTGCAAA
TCAAAGAACTGCTCCTCAGTGGATGTTGCCTTTACTTCTAGGCCTGTACGGAAGT
GTTACTTCTGCTCTAAAAGCTGCGGAATTTGTACCCGCGG
SEQ ID NO: 11-HSP70 Promoter
ACTAGTCCTGCAGGGCCGCCCACTCCCCCTTCCTCTCAGGGTCCCTGTCCCCTCCA
GTGAATCCCAGAAGACTCTGGAGAGTTCTGAGCAGGGGGCGGCACTCTGGCCTC
TGATTGGTCCAAGGAAGGCTGGGGGGCAGGACGGGAGGCGAAAACCCTGGAAT
ATTCCCGACCTGGCAGCCTCATCGAGCTCGGTGATTGGCTCAGAAGGGAAAAGG
CGGGTCTCCGTGACGACTTATAAAAGCCCAGGGGCAAGCGGTCCGGATAACGGC
TAGCCTGAGGAGCTGCTGCGACAGTCCACTACCTTTTTCGAGAGTGACTCCCGTT
GTCCCAAGGCTTCCCAGAGCGAACCTGTGCGGCTGCAGGCACCGGCGCGTCGAG
TTTCCGGCGTCCGGAAGGACCGAGCTCTTCTCGCGGATCCAGTGTTCCGTTTCCA
GCCCCCAATCTCAGAGCGGAGCCGACAGAGAGCAGGGAACC
SEQ ID NO: 12-sCBA Promoter
ACTAGTCCCCACGTTCTGCTTCACTCTCCCCATCTCCCCCCCCTCCCCACCCCCAA
TTTTGTATTTATTTATTTTTTAATTATTTTGTGCAGCGATGGGGGCGGGGGGGGGG
GGGGGGCGCGCGCCAGGCGGGGCGGGGCGGGGCGAGGGGCGGGGCGGGGCGA
GGCGGAGAGGTGCGGCGGCAGCCAATCAGAGCGGCGCGCTCCGAAAGTTTCCTT
TTATGGCGAGGCGGCGGCGGCGGCGGCCCTATAAAAAGCGAAGCGCGCGGCGG
G
SEQ ID NO: 13-alpha1 antitrypsin, SERPINA1 Promoter
GTTAACGGCTGCCCACTGGGCATTTCATAGGTGGCTCAGTCCTCTTCCCTCTGCA
GCTGGCCCCAGAAACCTGCCAGTTATTGGTGCCAGGTCTGTGCCAGGAGGGCGA
GGCCTGTCATTTCTAGTAATCCTCTGGGCAGTGTGACTGTACCTCTTGCGGCAAC
TCAAAGGGAGAGGGTGACTTGTCCCGGGTCACAGAGCTGAAAGGGCAGGTACAA
CAGGTGACATGCCGGGCTGTCTGAGTTTATGAGGGCCCAGTCTTGTGTCTGCCGG
GCAATGAGCAAGGCTCCTTCCTGTCCAAGCTCCCCGCCCCTCCCCAGCCTACTGC
CTCCACCCGAAGTCTACTTCCTGGG
SEQ ID NO: 14-Representative CFI AAV Vector (with alpha1 antitrypsin, SERPINA1
Promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTGTTAACG
GCTGCCCACTGGGCATTTCATAGGTGGCTCAGTCCTCTTCCCTCTGCAGCTGGCC
CCAGAAACCTGCCAGTTATTGGTGCCAGGTCTGTGCCAGGAGGGCGAGGCCTGT
CATTTCTAGTAATCCTCTGGGCAGTGTGACTGTACCTCTTGCGGCAACTCAAAGG
GAGAGGGTGACTTGTCCCGGGTCACAGAGCTGAAAGGGCAGGTACAACAGGTGA
CATGCCGGGCTGTCTGAGTTTATGAGGGCCCAGTCTTGTGTCTGCCGGGCAATGA
GCAAGGCTCCTTCCTGTCCAAGCTCCCCGCCCCTCCCCAGCCTACTGCCTCCACC
CGAAGTCTACTTCCTGGGACCGGTCTCGAAGGCCTGCAGGCGGCCGCCGCCACC
ATGAAGCTTCTTCATGTTTTCCTGTTATTTCTGTGCTTCCACTTAAGGTTTTGCAA
GGTCACTTATACATCTCAAGAGGATCTGGTGGAGAAAAAGTGCTTAGCAAAAAA
ATATACTCACCTCTCCTGCGATAAAGTCTTCTGCCAGCCATGGCAGAGATGCATT
GAGGGCACCTGTGTTTGTAAACTACCGTATCAGTGCCCAAAGAATGGCACTGCA
GTGTGTGCAACTAACAGGAGAAGCTTCCCAACATACTGTCAACAAAAGAGTTTG
GAATGTCTTCATCCAGGGACAAAGTTTTTAAATAACGGAACATGCACAGCCGAA
GGAAAGTTTAGTGTTTCCTTGAAGCATGGAAATACAGATTCAGAGGGAATAGTT
GAAGTAAAACTTGTGGACCAAGATAAGACAATGTTCATATGCAAAAGCAGCTGG
AGCATGAGGGAAGCCAACGTGGCCTGCCTTGACCTTGGGTTTCAACAAGGTGCT
GATACTCAAAGAAGGTTTAAGTTGTCTGATCTCTCTATAAATTCCACTGAATGTC
TACATGTGCATTGCCGAGGATTAGAGACCAGTTTGGCTGAATGTACTTTTACTAA
GAGAAGAACTATGGGTTACCAGGATTTCGCTGATGTGGTTTGTTATACACAGAAA
GCAGATTCTCCAATGGATGACTTCTTTCAGTGTGTGAATGGGAAATACATTTCTC
AGATGAAAGCCTGTGATGGTATCAATGATTGTGGAGACCAAAGTGATGAACTGT
GTTGTAAAGCATGCCAAGGCAAAGGCTTCCATTGCAAATCGGGTGTTTGCATTCC
AAGCCAGTATCAATGCAATGGTGAGGTGGACTGCATTACAGGGGAAGATGAAGT
TGGCTGTGCAGGCTTTGCATCTGTGACTCAAGAAGAAACAGAAATTTTGACTGCT
GACATGGATGCAGAAAGAAGACGGATAAAATCATTATTACCTAAACTATCTTGT
GGAGTTAAAAACAGAATGCACATTCGAAGGAAACGAATTGTGGGAGGAAAGCG
AGCACAACTGGGAGACCTCCCATGGCAGGTGGCAATTAAGGATGCCAGTGGAAT
CACCTGTGGGGGAATTTATATTGGTGGCTGTTGGATTCTGACTGCTGCACATTGT
CTCAGAGCCAGTAAAACTCATCGTTACCAAATATGGACAACAGTAGTAGACTCG
ATACACCCCGACCTTAAACGTATAGTAATTGAATACGTGGATAGAATTATTTTCC
ATGAAAACTACAATGCAGGCACTTACCAAAATGACATCGCTTTGATTGAAATGA
AAAAAGACGGAAACAAAAAAGATTGTGAGCTGCCTCGTTCCATCCCTGCCTGTG
TCCCCTGGTCTCCTTACCTATTCCAACCTAATGATACATGCATCGTTTCTGGCTGG
GGACGAGAAAAAGATAACGAAAGAGTCTTTTCACTTCAGTGGGGTGAAGTTAAA
CTAATAAGCAACTGCTCTAAGTTTTACGGAAATCGTTTCTATGAAAAAGAAATGG
AATGTGCAGGTACATATGATGGTTCCATCGATGCCTGTAAAGGGGACTCTGGAG
GCCCCTTAGTCTGTATGGATGCCAACAATGTGACTTATGTCTGGGGTGTTGTGAG
TTGGGGGGAAAACTGTGGAAAACCAGAGTTCCCAGGTGTTTACACCAAAGTGGC
CAATTATTTTGACTGGATTAGCTACCATGTAGGAAGGCCTTTTATTTCTCAGTACA
ATGTATAATAAGATATCGATACATTGATGAGTTTGGACAAACCACAACTAGAAT
GCAGTGAAAAAAATGCTTTATTTGTGAAATTTGTGATGCTATTGCTTTATTTGTAA
CCATTATAAGCTGCAATAAACAAGATATCGTTAACTCGAGGGATCCCACGTGCTG
ATTTTGTAGGTAACCACGTGCGGACCGAGCGGCCGCAGGAACCCCTAGTGATGG
AGTTGGCCACTCCCTCTCTGCGCGCTCGCTCGCTCACTGAGGCCGGGCGACCAAA
GGTCGCCCGACGCCCGGGCTTTGCCCGGGCGGCCTCAGTGAGCGAGCGAGCGCG
CAGCTGCCTGCAGGGGCGCCTGATGCGGTATTTTCTCCTTACGCATCTGTGCGGT
ATTTCACACCGCATACGTCAAAGCAACCATAGTACGCGCCCTGTAGCGGCGCATT
AAGCGCGGCGGGTGTGGTGGTTACGCGCAGCGTGACCGCTACACTTGCCAGCGC
CCTAGCGCCCGCTCCTTTCGCTTTCTTCCCTTCCTTTCTCGCCACGTTCGCCGGCTT
TCCCCGTCAAGCTCTAAATCGGGGGCTCCCTTTAGGGTTCCGATTTAGTGCTTTAC
GGCACCTCGACCCCAAAAAACTTGATTTGGGTGATGGTTCACGTAGTGGGCCATC
GCCCTGATAGACGGTTTTTCGCCCTTTGACGTTGGAGTCCACGTTCTTTAATAGTG
GACTCTTGTTCCAAACTGGAACAACACTCAACCCTATCTCGGGCTATTCTnTGAT
TTATAAGGGATTTTGCCGATTTCGGCCTATTGGTTAAAAAATGAGCTGATTTAAC
AAAAATTTAACGCGAATTTTAACAAAATATTAACGTTTACAATTTTATGGTGCAC
TCTCAGTACAATCTGCTCTGATGCCGCATAGTTAAGCCAGCCCCGACACCCGCCA
ACACCCGCTGACGCGCCCTGACGGGCTTGTCTGCTCCCGGCATCCGCTTACAGAC
AAGCTGTGACCGTCTCCGGGAGCTGCATGTGTCAGAGGTTTTCACCGTCATCACC
GAAACGCGCGAGACGAAAGGGCCTCGTGATACGCCTATTTTTATAGGTTAATGTC
ATGATAATAATGGTTTCTTAGACGTCAGGTGGCACTTTTCGGGGAAATGTGCGCG
GAACCCCTATTTGTTTATTTTTCTAAATACATTCAAATATGTATCCGCTCATGAGA
CAATAACCCTGATAAATGCTTCAATAATATTGAAAAAGGAAGAGTATGAGTATT
CAACATTTCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTC
GCCCCGAAGAACGTTTTCCAATGATGAGCACTTTTAAAGTTCTGCTATGTGGCGC
GGTATTATCCCGTATTGACGCCGGGCAAGAGCAACTCGGTCGCCGCATACACTAT
TCTCAGAATGACTTGGTTGAGTACTCACCAGTCACAGAAAAGCATCTTACGGATG
GCATGACAGTAAGAGAATTATGCAGTGCTGCCATAACCATGAGTGATAACACTG
CGGCCAACTTACTTCTGACAACGATCGGAGGACCGAAGGAGCTAACCGCTTTTTT
GCACAACATGGGGGATCATGTAACTCGCCTTGATCGTTGGGAACCGGAGCTGAA
TGAAGCCATACCAAACGACGAGCGTGACACCACGATGCCTGTAGCAATGGCAAC
AACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCC
CTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTAT
CTACACGACGGGGAGTCAGGCAACTATGGATGAACGAAATAGACAGATCGCTGA
GATAGGTGCCTCACTGATTAAGCATTGGTAACTGTCAGACCAAGTTTACTCATAT
ATACTTTAGATTGATTTAAAACTTCATTTTTAATTTAAAAGGATCTAGGTGAAGA
TCCTTTTTGATAATCTCATGACCAAAATCCCTTAACGTGAGTTTTCGTTCCACTGA
GCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTT
GCCGGATCAAGAGCTACCAACTCTTTTTCCGAAGGTAACTGGCTTCAGCAGAGCG
CAGATACCAAATACTGTCCTTCTAGTGTAGCCGTAGTTAGGCCACCACTTCAAGA
ACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCG
GATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGC
GCCACGCTTCCCGAAGGGAGAAAGGCGGACAGGTATCCGGTAAGCGGCAGGGTC
GGAACAGGAGAGCGCACGAGGGAGCTTCCAGGGGGAAACGCCTGGTATCTTTAT
AGTCCTGTCGGGTTTCGCCACCTCTGACTTGAGCGTCGATTTTGTGATGCTCGTC
AGGGGGGCGGAGCCTATGGAAAAACGCCAGCAACGCGGCCTTTTTACGGTTCCT
GGCCTTTTGCTGGCCTTTTGCTCACATGT
SEQ ID NO: 15-ALB Promoter
GTTAACCAGTTCCAGATGGTAAATATACACAAGGGATTTAGTCAAACAATTTTTT
GGCAAGAATATTATGAATTTTGTAATCGGTTGGCAGCCAATGAAATACAAAGAT
GAGTCTAGTTAATAATCTACAATTATTGGTTAAAG
SEQ ID NO: 16-Representative CFI AAV Vector (with ALB Promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTGTTAACC
AGTTCCAGATGGTAAATATACACAAGGGATTTAGTCAAACAATTTTTTGGCAAGA
ATATTATGAATTTTGTAATCGGTTGGCAGCCAATGAAATACAAAGATGAGTCTAG
TTAATAATCTACAATTATTGGTTAAAGACCGGTCTCGAAGGCCTGCAGGCGGCCG
CCGCCACCATGAAGCTTCTTCATGTTTTCCTGTTATTTCTGTGCTTCCACTTAAGG
TTTTGCAAGGTCACTTATACATCTCAAGAGGATCTGGTGGAGAAAAAGTGCTTAG
CAAAAAAATATACTCACCTCTCCTGCGATAAAGTCTTCTGCCAGCCATGGCAGAG
ATGCATTGAGGGCACCTGTGTTTGTAAACTACCGTATCAGTGCCCAAAGAATGGC
ACTGCAGTGTGTGCAACTAACAGGAGAAGCTTCCCAACATACTGTCAACAAAAG
AGTTTGGAATGTCTTCATCCAGGGACAAAGTTTTTAAATAACGGAACATGCACAG
CCGAAGGAAAGTTTAGTGTTTCCTTGAAGCATGGAAATACAGATTCAGAGGGAA
TAGTTGAAGTAAAACTTGTGGACCAAGATAAGACAATGTTCATATGCAAAAGCA
GCTGGAGCATGAGGGAAGCCAACGTGGCCTGCCTTGACCTTGGGTTTCAACAAG
GTGCTGATACTCAAAGAAGGTTTAAGTTGTCTGATCTCTCTATAAATTCCACTGA
ATGTCTACATGTGCATTGCCGAGGATTAGAGACCAGTTTGGCTGAATGTACTTTT
ACTAAGAGAAGAACTATGGGTTACCAGGATTTCGCTGATGTGGTTTGTTATACAC
AGAAAGCAGATTCTCCAATGGATGACTTCTTTCAGTGTGTCAATGGGAAATACAT
TTCTCAGATGAAAGCCTGTGATGGTATCAATGATTGTGGAGACCAAAGTGATGA
ACTGTGTTGTAAAGCATGCCAAGGCAAAGGCTTCCATTGCAAATCGGGTGTTTGC
ATTCCAAGCCAGTATCAATGCAATGGTGAGGTGGACTGCATTACAGGGGAAGAT
GAAGTTGGCTGTGCAGGCTTTGCATCTGTGACTCAAGAAGAAACAGAAATTTTGA
CTGCTGACATGGATGCAGAAAGAAGACGGATAAAATCATTATTACCTAAACTAT
CTTGTGGAGTTAAAAACAGAATGCACATTCGAAGGAAACGAATTGTGGGAGGAA
AGCGAGCACAACTGGGAGACCTCCCATGGCAGGTGGCAATTAAGGATGCCAGTG
GAATCACCTGTGGGGGAATTTATATTGGTGGCTGTTGGATTCTCACTGCTGCAGA
TTGTCTCAGAGCCAGTAAAACTCATCGTTACCAAATATGGACAACAGTAGTAGA
CTGGATACACCCCGACCTTAAACGTATAGTAATTGAATACGTGGATAGAATTATT
TTCCATGAAAACTACAATGCAGGCACTTACCAAAATGACATCGCTTTGATTGAAA
TGAAAAAAGACGGAAACAAAAAAGATTGTGAGCTGCCTCGTTCCATCCCTGCCT
GTGTCCCCTGGTCTCCTTACCTATTCCAACCTAATGATACATGCATCGTTTCTGGC
TGGGGACGAGAAAAAGATAACGAAAGAGTCTTTTCACTTCAGTGGGGTGAAGTT
AAACTAATAAGCAACTGCTCTAAGTTTTACGGAAATCGTTTCTATGAAAAAGAAA
TGGAATGTGCAGGTACATATGATGGTTCCATCGATGCCTGTAAAGGGGACTCTGG
AGGCCCCTTAGTCTGTATGGATGCCAACAATGTGACTTATGTCTGGGGTGTTGTG
AGTTGGGGGGAAAACTGTGGAAAACCAGAGTTCCCAGGTGTTTACACCAAAGTG
GCCAATTATTTTGACTGGATTAGCTACCATGTAGGAAGGCCTTTTATTTCTCAGTA
CAATGTATAATAAGATATCGATACATTGATGAGTTTGGACAAACCACAACTAGA
ATGCAGTGAAAAAAATGCTTTATTTGTGAAATTTGTGATGCTATTGCTTTATTTGT
AACCATTATAAGCTGCAATAAACAAGATATCGTTAACTCGAGGGATCCCACGTG
CTGATTTTGTAGGTAACCACGTGCGGACCGAGCGGCCGCAGGAACCCCTAGTGA
TGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGC
GCGCAGCTGCCTGCAGGGGCGCCTGATGCGGTATTTTCTCCTTACGCATCTGTGC
GGTATTTCACACCGCATACGTCAAAGCAACCATAGTACGCGCCCTGTAGCGGCG
CATTAAGCGCGGCGGGTGTGGTGGTTACGCGCAGCGTGACCGCTACACTTGCCA
GCGCCCTAGCGCCCGCTCCTTTCGCTTTCTTCCCTTCCTTTCTCGCCACGTTCGCC
GGCTTTCCCCGTCAAGCTCTAAATCGGGGGCTCCCTTTAGGGTTCCGATTTAGTG
CTTTACGGCACCTCGACCCCAAAAAACTTGATTTGGGTGATGGTTCACGTAGTGG
GCCATCGCCCTGATAGACGGTTTTTCGCCCTTTGACGTTGGAGTCCACGTTCTTTA
ATAGTGGACTCTTGTTCCAAACTGGAACAACACTCAACCCTATCTCGGGCTATTC
TTTTGATTTATAAGGGATTTTGCCGATTTCGGCCTATTGGTTAAAAAATGAGCTG
ATTTAACAAAAATTTAACGCGAATTTTAACAAAATATTAACGTTTACAATTTTAT
GGTGCACTCTCAGTACAATCTGCTCTGATGCCGCATAGTTAAGCCAGCCCCGACA
CCCGCCAACACCCGCTGACGCGCCCTGACGGGCTTGTCTGCTCCCGGCATCCGCT
TACAGACAAGCTGTGACCGTCTCCGGGAGCTGCATGTGTCAGAGGTTTTCACCGT
CATCACCGAAACGCGCGAGACGAAAGGGCCTCGTGATACGCCTATTTTTATAGG
TTAATGTCATGATAATAATGGTTTCTTAGACGTCAGGTGGCACTTTTCGGGGAAA
TGTGCGCGGAACCCCTATTTGTTTATTTTTCTAAATACATTCAAATATGTATCCGC
TCATGAGACAATAACCCTGATAAATGCTTCAATAATATTGAAAAAGGAAGAGTA
TGAGTATTCAACATTTCCGTGTCGCCCTTATTCCCTTTTTTGCGGCATTTTGCCTTC
CTGTTTTTGCTCACCCAGAAACGCTGGTGAAAGTAAAAGATGCTGAAGATCAGTT
GGGTGCACGAGTGGGTTACATCGAACTGGATCTCAACAGCGGTAAGATCCTTGA
GAGTTTTCGCCCCGAAGAACGTTTTCCAATGATGAGCACTTTTAAAGTTCTGCTA
TGTGGCGCGGTATTATCCCGTATTGACGCCGGGCAAGAGCAACTCGGTCGCCGC
ATACACTATTCTCAGAATGACTTGGTTGAGTACTCACCAGTCACAGAAAAGCATC
TTACGGATGGCATGACAGTAAGAGAATTATGCAGTGCTGCCATAACCATGAGTG
ATAACACTGCGGCCAACTTACTTCTGACAACGATCGGAGGACCGAAGGAGCTAA
CCGCTTTTTTGCACAACATGGGGGATCATGTAACTCGCCTTGATCGTTGGGAACC
GGAGCTGAATGAAGCCATACCAAACGACGAGCGTGACACCACGATGCCTGTAGC
AATGGCAACAACGTTGCGCAAACTATTAACTGGCGAACTACTTACTCTAGCTTCC
CGGCAACAATTAATAGACTGGATGGAGGCGGATAAAGTTGCAGGACCACTTCTG
CGCTCGGCCCTTCCGGCTGGCTGGTTTATTGCTGATAAATCTGGAGCCGGTGAGC
GTGGGTCTCGCGGTATCATTGCAGCACTGGGGCCAGATGGTAAGCCCTCCCGTAT
CGTAGTTATCTACACGACGGGGAGTCAGGCAACTATGGATGAACGAAATAGACA
GATCGCTGAGATAGGTGCCTCACTGATTAAGCATTGGTAACTGTCAGACCAAGTT
TACTCATATATACTTTAGATTGATTTAAAACTTCATTTTTAATTTAAAAGGATCTA
GGTGAAGATCCTTTTTGATAATCTCATGACCAAAATCCCTTAACGTGAGTTTTCGT
TCCACTGAGCGTCAGACCCCGTAGAAAAGATCAAAGGATCTTCTTGAGATCCTTT
TTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCA
GCAGAGCGCAGATACCAAATACTGTCCTTCTAGTGTAGCCGTAGTTAGGCCACCA
CTTCAAGAACTCTGTAGCACCGCCTACATACCTCGCTCTGCTAATCCTGTTACCA
GTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTAT
GAGAAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGCCTG
GTATCTTTATAGTCCTGTCGGGTTTCGCCACCTCTGACTTGAGCGTCGATTTTTGT
GATGCTCGTCAGGGGGGCGGAGCCTATGGAAAAACGCCAGCAACGCGGCCTTTT
TACGGTTCCTGGCCTTTTGCTGGCCTTTTGCTCACATGT
SEQ ID NO: 17-CAG Promoter
GTTAACTTGGCAAAGAATTCTGCAGTCGACGGTACCGCGGGCCCGGGATCCACC
GGTCGCCACCATGGTGCGCTCCTCCAAGAACGTCATCAAGGAGTTCATGCGCTTC
AAGGTGCGCATGGAGGGCACCGTGAACGGCCACGAGTTCGAGATCGAGGGCGA
GGGCGAGGGCCGCCCCTACGAGGGCCACAACACCGTGAAGCTGAAGGTGACCA
AGGGCGGCCCCCTGCCCTTCGCCTGGGACATCCTGTCCCCCCAGTTCCAGTACGG
CTCCAAGGTGTACGTGAAGCACCCCGCCGACATCCCCGACTACAAGAAGCTGTC
CTTCCCCGAGGGCTTCAAGTGGGAGCGCGTGATGAACTTCGAGGACGGCGGCGT
GGTGACCGTGACCCAGGACTCCTCCCTGCAGGACGGCTGCTTCATCTACAAGGTG
AAGTTCATCGGCGTGAACTTCCCCTCCGACGGCCCCGTAATGCAGAAGAAGACC
ATGGGCTGGGAGGCCTCCACCGAGCGCCTGTACCCCCGCGACGGCGTGCTGAAG
GGCGAGATCCACAAGGCCCTGAAGCTGAAGGACGGCGGCCACTACCTGGTGGAG
TTCAAGTCCATCTACATGGCCAAGAAGCCCGTGCAGCTGCCCGGCTACTACTACG
TGGACTCCAAGCTGGACATCACCTCCCACAACGAGGACTACACCATCGTGGAGC
AGTACGAGCGCACCGAGGGCCGCCACCACCTGTTCCTGTAGCGGCCGCACTCCTC
AGGTGCAGGCTGCCTATCAGAAGGTGGTGGCTGGTGTGGCCAATGCCCTGGCTC
ACAAATACCACTGAGATCTTTTTCCCTCTGCCAAAAATTATGGGGACATCATGAA
GCCCCTTGAGCATCTGACTTCTGGCTAATAAAGGAAATTTATTTTCATTGCAATAG
TGTGTTGGAATTTTTTGTGTCTCTCACTCGGAAGGACATATGGGAGGGCAAATC
SEQ ID NO: 18-Representative CFI AAV Vector (with CAG Promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTGTTAACTT
GGCAAAGAATTCTGCAGTCGACGGTACCGCGGGCCCGGGATCCACCGGTCGCCA
CCATGGTGCGCTCCTCCAAGAACGTCATCAAGGAGTTCATGCGCTTCAAGGTGCG
CATGGAGGGCACCGTGAACGGCCACGAGTTCGAGATCGAGGGCGAGGGCGAGG
GCCGCCCCTACGAGGGCCACAACACCGTGAAGCTGAAGGTGACCAAGGGCGGCC
CCCTGCCCTTCGCCTGGGACATCCTGTCCCCCCAGTTCCAGTACGGCTCCAAGGT
GTACGTGAAGCACCCCGCCGACATCCCCGACTACAAGAAGCTGTCCTTCCCCGA
GGGCTTCAAGTGGGAGCGCGTGATGAACTTCGAGGACGGCGGCGTGGTGACCGT
GACCCAGGACTCCTCCCTGCAGGACGGCTGCTTCATCTACAAGGTGAAGTTCATC
GGCGTGAACTTCCCCTCCGACGGCCCCGTAATGCAGAAGAAGACCATGGGCTGG
GAGGCCTCCACCGAGCGCCTGTACCCCCGCGACGGCGTGCTGAAGGGCGAGATC
CACAAGGCCCTGAAGCTGAAGGACGGCGGCCACTACCTGGTGGAGTTCAAGTCC
ATCTACATGGCCAAGAAGCCCGTGCAGCTGCCCGGCTACTACTACGTGGACTCCA
AGCTGGACATCACCTCCCACAACGAGGACTACACCATCGTGCAGCAGTACGAGC
GCACCGAGGGCCGCCACCACCTGTTCCTGTAGCGGCCGCACTCCTCAGGTGCAG
GCTGCCTATCAGAAGGTGGTGGCTGGTGTGGCCAATGCCCTGGCTCACAAATACC
ACTGAGATCTTTTTCCCTCTGCCAAAAATTATGGGGACATCATGAAGCCCCTTGA
GCATCTGACTTCTGGCTAATAAAGGAAATTTATTTTCATTGCAATAGTGTGTTGGA
ATTTTTTGTGTCTCTCACTCGGAAGGACATATGGGAGGGCAAATCACCGGTCTCG
AAGGCCTGCAGGCGGCCGCCGCCACCATGAAGCTTCTTCATGTTTTCCTGTTATT
TCTGTGCTTCCACTTAAGGTTTTGCAAGGTCACTTATACATCTCAAGAGGATCTG
GTGGAGAAAAAGTGCTTAGCAAAAAAATATACTCACCTCTCCTGCGATAAAGTC
TTCTGCCAGCCATGGCAGAGATGCATTGAGGGCACCTGTGTTTGTAAACTACCGT
ATCAGTGCCCAAAGAATGGCACTGCAGTGTGTGCAACTAACAGGAGAAGCTTCC
CAACATACTGTCAACAAAAGAGTTTGGAATGTCTTCATCCAGGGACAAAGTTTTT
AAATAACGGAACATGCACAGCCGAAGGAAAGTTTAGTGTTTCCTTGAAGCATGG
AAATACAGATTCAGAGGGAATAGTTGAAGTAAAACTTGTGGACCAAGATAAGAC
AATGTTCATATGCAAAAGCAGCTGGAGCATGAGGGAAGCCAACGTGGCCTGCCT
TGACCTTGGGTTTCAACAAGGTGCTGATACTCAAAGAAGGTTTAAGTTGTCTGAT
CTCTCTATAAATTCCACTGAATGTCTACATGTGCATTGCCGAGGATTAGAGACCA
GTTTGGCTGAATGTACTTTTACTAAGAGAAGAACTATGGGTTACCAGGATTTCGC
TGATGTGGTTTGTTATACACAGAAAGCAGATTCTCCAATGGATGACTTCTTTCAG
TGTGTGAATGGGAAATACATTTCTCAGATGAAAGCCTGTGATGGTATCAATGATT
GTGGAGACCAAAGTGATGAACTGTGTTGTAAAGCATGCCAAGGCAAAGGCTTCC
ATTGCAAATCGGGTGTTTGCATTCCAAGCCAGTATCAATGCAATGGTGAGGTGGA
CTGCATTACAGGGGAAGATGAAGTTGGCTGTGCAGGCTTTGCATCTGTGACTCAA
GAAGAAACAGAAATTTTGACTGCTGACATGGATGCAGAAAGAAGACGGATAAA
ATCATTATTACCTAAACTATCTTGTGGAGTTAAAAACAGAATGCACATTCGAAGG
AAACGAATTCTGGGAGGAAAGCGAGCACAACTGGGAGACCTCCCATGGCAGGT
GGCAATTAAGGATGCCAGTGGAATCACCTGTGGGGGAATTTATATTGGTGGCTGT
TGGATTCTGACTGCTGCACATTGTCTCAGAGCCAGTAAAACTCATCGTTACCAAA
TATGGACAACAGTAGTAGACTGGATACACCCCGACCTTAAACGTATAGTAATTG
AATACGTGGATAGAATTATTTTCCATGAAAACTACAATGCAGGCACTTACCAAA
ATGACATCGCTTTGATTGAAATGAAAAAAGACGGAAACAAAAAAGATTGTGAGC
TGCCTCGTTCCATCCCTGCCTGTGTCCCCTGGTCTCCTTACCTATTCCAACCTAAT
GATACATGCATCGTTTCTGGCTGGGGACGAGAAAAAGATAACGAAAGAGTCTTT
TCACTTCAGTGGGGTGAAGTTAAACTAATAAGCAACTGCTCTAAGTTTTACGGAA
ATCGTTTCTATGAAAAAGAAATGGAATGTGCAGGTACATATGATGGTTCCATCGA
TGCCTGTAAAGGGGACTCTGGAGGCCCCTTAGTCTGTATGGATGCCAACAATGTG
ACTTATGTCTGGGGTGTTGTGAGTTGGGGGGAAAACTGTGGAAAACCAGAGTTC
CCAGGTGTTTACACCAAAGTGGCCAATTATTTTGACTGGATTAGCTACCATGTAG
GAAGGCCTTTTATTTCTCAGTACAATGTATAATAAGATATCGATACATTGATGAG
TTTGGACAAACCACAACTAGAATGCAGTGAAAAAAATGCTTTATTTGTGAAATTT
GTGATGCTATTGCTTTATTTGTAACCATTATAAGCTGCAATAAACAAGATATCGT
TAACTCGAGGGATCCCACGTGCTGATTTTGTAGGTAACCACGTGCGGACCGAGC
GGCCGCAGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCCCGGGC
GGCCTCAGTGAGCGAGCGAGCGCGCAGCTGCCTGCAGGGGCGCCTGATGCGGTA
TTTTCTCCTTACGCATCTGTGCGGTATTTCACACCGCATACGTCAAAGCAACCAT
AGTACGCGCCCTGTAGCGGCGCATTAAGCGCGGCGGGTGTGGTGGTTACGCGCA
GCGTGACCGCTACACTTGCCAGCGCCCTAGCGCCCGCTCCTTTCGCTTTCTTCCCT
TCCTTTCTCGCCACGTTCGCCGGCTTTCCCCGTCAAGCTCTAAATCGGGGGCTCCC
TTTAGGGTTCCGATTTAGTGCTTTACGGCACCTCGACCCCAAAAAACTTGATTTG
GGTGATGGTTCACGTAGTGGGCCATCGCCCTGATAGACGGTTTTTCGCCCTTTGA
CGTTGGAGTCCACGTTCTTTAATAGTGGACTCTTGTTCCAAACTGGAACAACACT
CAACCCTATCTCGGGCTATTCTTTTGATTTATAAGGGATTTTGCCGATTTCGGCCT
ATTGGTTAAAAAATGAGCTGATTTAACAAAAATTTAACGCGAATTTTAACAAAAT
ATTAACGTTTACAATTTTATGGTGCACTCTCAGTACAATCTGCTCTGATGCCGCAT
AGTTAAGCCAGCCCCGACACCCGCCAACACCCGCTGACGCGCCCTGACGGGCTT
GTCTGCTCCCGGCATCCGCTTACAGACAAGCTGTGACCGTCTCCGGGAGCTGCAT
GTGTCAGAGGTTTTCACCGTCATCACCGAAACGCGCGAGACGAAAGGGCCTCGT
GATACGCCTATTTTTATAGGTTAATGTCATGATAATAATGGTTTCTTAGACGTCAG
GTGGCACTTTTCGGGGAAATGTGCGCGGAACCCCTATTTGTTTATTTTTCTAAATA
CATTCAAATATGTATCCGCTCATGAGACAATAACCCTGATAAATGCTTCAATAAT
ATTGAAAAAGGAAGAGTATGAGTATTCAACATTTCCGTGTCGCCCTTATTCCCTT
TTTTGCGGCATTTTGCCTTCCTGTTTTTGCTCACCCAGAAACGCTGGTGAAAGTAA
AAGATGCTGAAGATCAGTTGGGTGCACGAGTGGGTTACATCGAACTGGATCTCA
ACAGCGGTAAGATCCTTGAGAGTTTTCGCCCCGAAGAACGTTTTCCAATGATGAG
CACTTTTAAAGTTCTGCTATGTGGCGCGGTATTATCCCGTATTGACGCCGGGCAA
GAGCAACTCGGTCGCCGCATACACTATTCTCAGAATGACTTGGTTGAGTACTCAC
CAGTCACAGAAAAGCATCTTACGGATGGCATGACAGTAAGAGAATTATGCAGTG
CTGCCATAACCATGAGTGATAACACTGCGGCCAACTTACTTCTGACAACGATCGG
AGGACCGAAGGAGCTAACCGCTTTTTTGCACAACATGGGGGATCATGTAACTCG
CCTTGATCGTTGGGAACCGGAGCTGAATGAAGCCATACCAAACGACGAGCGTGA
CACCACGATGCCTGTAGCAATGGCAACAACGTTGCGCAAACTATTAACTGGCGA
ACTACTTACTCTAGCTTCCCGGCAACAATTAATAGACTGGATGGAGGCGGATAA
AGTTGCAGGACCACTTCTGCGCTCGGCCCTTCCGGCTGGCTGGTTTATTGCTGAT
AAATCTGGAGCCGGTGAGCGTGGGTCTCGCGGTATCATTGCAGCACTGGGGCCA
GATGGTAAGCCCTCCCGTATCGTAGTTATCTACACGACGGGGAGTCAGGCAACT
ATGGATGAACGAAATAGACAGATCGCTGAGATAGGTGCCTCACTGATTAAGCAT
TGGTAACTGTCAGACCAAGTTTACTCATATATAGTTTAGATTGATTTAAAACTTCA
TTTTTAATTTAAAAGGATCTAGGTGAAGATCCTTTTTGATAATCTCATGACCAAA
ATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAA
AAACCACCGCTACCAGCGGTGGTTTGTTTGCCGGATCAAGAGCTACCAACTCTTT
TTCGGAAGGTAACTGGCTTCAGCAGAGCGCAGATACCAAATACTGTCCTTCTAGT
GTAGCCGTAGTTAGGCCACCACTTCAAGAACTCTGTAGCACCGCCTACATACCTC
GCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAA
CGGGGGGTTCGTGCACACAGCCCAGCTTGGAGCGAACGACCTACACCGAACTGA
GATACCTACAGCGTGAGCTATGAGAAAGCGCCACGCTTCCCGAAGGGAGAAAGG
CGGACAGGTATCCGGTAAGCGGCAGGGTCGGAACAGGAGAGCGCACGAGGGAG
CTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAA
ACGCCAGCAACGCGGCCTTTTTACGGTTCCTGGCCTTTTGCTGGCCTTTTGCTCAC
ATGT
SEQ ID NO: 19-CBA Promoter
TAGTGGCCCGCCTGGCTGACCGCCCAACGACCCCCGCCCATTGACGTCAATAATG
ACGTATGTTCCCATAGTAACGCCAATAGGGACTTTCCATTGACGTCAATGGGTGG
ACTATTTACGGTAAACTGCCCACTTGGCAGTACATCAAGTGTATCATATGCCAAG
TACGCCCCCTATTGACGTCAATGACGGTAAATGGCCCGCCTGGCATTATGCCCAG
TACATGACCTTATGGGACTTTCCTACTTGGCAGTACATCTACGTATTAGTCATCGC
TATTACCATGGTCGAGGTGAGCCCCACGTTCTGCTTCACTCTCCCCATCTCCCCCC
CCTCCCCACCCCCAATTTTGTATTTATTTATTTTTTAATTATTTTGTGCAGCGATGG
GGGCGGGGGGGGGGGGGGGGCGCGCGCCAGGCGGGGCGGGGCGGGGCGAGGG
GCGGGGCGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGC
GAAGCGCGCGGCGGGCGGGAGTCGCTGCGACGCTGCCTTCGCCCCGTGCCCCGC
TCCGCCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGTTTA
ATGACGGCTTGTTTCTTTTCTGTGGCTGCGTGAAAGCCTTGAGGGGCTCCGGGAG
GGCCCTTTGTGCGGGGGGGAGCGGCTCGGGGGGTGCGTGCGTGTGTGTGTGCGT
GGGGAGCGCCGCGTGCGGCCCGCGCTGCCCGGCGGCTGTGAGCGCTGCGGGCGC
GGCGCGGGGCTTTGTGCGCTCCGCAGTGTGCGCGAGGGGAGCGCGGCCGGGGGC
GGTGCCCCGCGGTGCGGGGGGGGCTGCGAGGGGAACAAAGGCTGCGTGCGGGG
TGTGTGCGTGGGGGGGTGAGCAGGGGGTGTGGGCGCGGCGGTCGGGCTGTAACC
CCCCCCTGCACCCCCCTCCCCGAGTTGCTGAGCACGGCCCGGCTTCGGGTGCGGG
GCTCCGTACGGGGCGTGGCGCGGGGCTCGCCGTGCCGGGCGGGGGGTGGCGGCA
GGTGGGGGTGCCGGGCGGGGCGGGGCCGCCTCGGGCCGGGGAGGGCTCGGGGG
AGGGGCGCGGCGGCCCCCGGAGCGCCGGCGGCTGTCGAGGCGCGGCGAGCCGC
AGCCATTGCCTTTTATGGTAATCGTGCGAGAGGGCGCAGGGACTTCCTTTGTCCC
AAATCTGTGCGGAGCCGAAATCTGGGAGGCGCCGCCGCACCCCCTCTAGCGGGC
GCGGGGCGAAGCGGTGCGGCGCCGGCAGGAAGGAAATGGGCGGGGAGGGCCTT
CGTGCGTCGCCGCGCCGCCGTCCCCTTCTCCCTCTCCAGCCTCGGGGCTGTCCGC
GGGGGGACGGCTGCCTTCGGGGGGGACGGGGCAGGGCGGGGTTCGGCTTCTGGC
GTGTGACCGGCGGCTCTAGAGCCTCTGCTAACCATGTTCATGCCTTCTTCTTTTTC
CTACAGCTCCTGGGCAACGTGCTGGTTATTGTGCTGTCTCATCATTTTGGCAAA
SEQ ID NO: 20-Representative CFI AAV Vector (with CBA Promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTGTTAACT
AGTGGCCCGCCTGGCTGACCGCCCAACGACCCCCGCCCATTGACGTCAATAATG
ACGTATGTTCCCATAGTAACGCCAATAGGGACTTTCCATTGACGTCAATGGGTGG
ACTATTTACGGTAAACTGCCCACTTGGCAGTACATCAAGTGTATCATATGCCAAG
TACGCCCCCTATTGACGTCAATGACGGTAAATGGCCCGCCTGGCATTATGCCCAG
TACATGACCTTATGGGACTTTCCTACTTGGCAGTACATCTACGTATTAGTCATCGC
TATTACCATGGTCGAGGTGAGCCCCACGTTCTGCTTCACTCTCCCCATCTCCCCCC
CCTCCCCACCCCCAATTTTGTATTTATTTATTTTTTAATTATTTTGTGCAGCGATGG
GGGCGGGGGGGGGGGGGGGGCGCGCGCCAGGCGGGGCGGGGCGGGGCGAGGG
GCGGGGCGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGC
GAAGCGCGCGGCGGGCGGGAGTCGCTGCGACGCTGCCTTCGCCCCGTGCCCCGC
TCCGCCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGTTTA
ATGACGGCTTGTTTCTTTTCTGTGGCTGCGTGAAAGCCTTGAGGGGCTCCGGGAG
GGCCCTTTGTGCGGGGGGGAGCGGCTCGGGGGGTGCGTGCGTGTGTGTGTGCGT
GGGGAGCGCCGCGTGCGGCCCGCGCTGCCCGGCGGCTGTGAGCGCTGCGGGCGC
GGCGCGGGGCTTTGTGCGCTCCGCAGTGTGCGCGAGGGGAGCGCGGCCGGGGGC
GGTGCCCCGCGGTGCGGGGGGGGCTGCGAGGGGAACAAAGGCTGCGTGCGGGG
TGTGTGCGTGGGGGGGTGAGCAGGGGGTGTGGGCGCGGCGGTCGGGCTGTAACC
CCCCCCTGCACCCCCCTCCCCGAGTTGCTGAGCACGGCCCGGCTTCGGGTGCGGG
GCTCCGTACGGGGCGTGGCGCGGGGCTCGCCGTGCCGGGCGGGGGGTGGCGGCA
GGTGGGGGTGCCGGGCGGGGCGGGGCCGCCTCGGGCCGGGGAGGGCTCGGGGG
AGGGGCGCGGCGGCCCCCGGAGCGCCGGCGGCTGTCGAGGCGCGGCGAGCCGC
AGCCATTGCCTTTTATGGTAATCGTGCGAGAGGGCGCAGGGACTTCCTTTGTCCC
AAATCTGTGCGGAGCCGAAATCTGGGAGGCGCCGCCGCACCCCCTCTAGCGGGC
GCGGGGCGAAGCGGTGCGGCGCCGGCAGGAAGGAAATGGGCGGGGAGGGCCTT
CGTGCGTCGCCGCGCCGCCGTCCCCTTCTCCCTCTCCAGCCTCGGGGCTGTCCGC
GGGGGGACGGCTGCCTTCGGGGGGGACGGGGCAGGGCGGGGTTCGGCTTCTGGC
GTGTGACCGGCGGCTCTAGAGCCTCTGCTAACCATGTTCATGCCTTCTTCTTTTTC
CTACAGCTCCTGGGCAACGTGCTGGTTATTGTGCTGTCTCATCATTTTGGCAAAA
CCGGTCTCGAAGGCCTGCAGGCGGCCGCCGCCACCATGAAGCTTCTTCATGTTTT
CCTGTTATTTCTGTGCTTCCACTTAAGGTTTTGCAAGGTCACTTATACATCTCAAG
AGGATCTGGTGGAGAAAAAGTGCTTAGCAAAAAAATATACTCACCTCTCCTGCG
ATAAAGTCTTCTGCCAGCCATGGCAGAGATGCATTGAGGGCACCTGTGTTTGTAA
ACTACCGTATCAGTGCCCAAAGAATGGCACTGCAGTGTGTGCAACTAACAGGAG
AAGCTTCCCAACATACTGTCAACAAAAGAGTTTGGAATGTCTTCATCCAGGGACA
AAGTTTTTAAATAACGGAACATGCACAGCCGAAGGAAAGTTTAGTGTTTCCTTGA
AGCATGGAAATACAGATTCAGAGGGAATAGTTGAAGTAAAACTTGTGGACCAAG
ATAAGACAATGTTCATATGCAAAAGCAGCTGGAGCATGAGGGAAGCCAACGTGG
CCTGCCTTGACCTTGGGTTTCAACAAGGTGCTGATACTCAAAGAAGGTTTAAGTT
GTCTGATCTCTCTATAAATTCCACTGAATGTCTACATGTGCATTGCCGAGGATTA
GAGACCAGTTTGGCTGAATGTACTTTTACTAAGAGAAGAACTATGGGTTACCAGG
ATTTCGCTGATGTGGTTTGTTATACACAGAAAGCAGATTCTCCAATGGATGACTT
CTTTCAGTGTGTGAATGGGAAATACATTTCTCAGATGAAAGCCTGTGATGGTATC
AATGATTGTGGAGACCAAAGTGATGAACTGTGTTGTAAAGCATGCCAAGGCAAA
GGCTTCCATTGCAAATCGGGTGTTTGCATTCCAAGCCAGTATCAATGCAATGGTG
AGGTGGACTGCATTACAGGGGAAGATGAAGTTGGCTGTGCAGGCTTTGCATCTGT
GACTCAAGAAGAAACAGAAATTTTGACTGCTGACATGGATGCAGAAAGAAGACG
GATAAAATCATTATTACCTAAACTATCTTGTGGAGTTAAAAACAGAATGCACATT
CGAAGGAAACGAATTGTGGGAGGAAAGCGAGCACAACTGGGAGACCTCCCATG
GCAGGTGGCAATTAAGGATGCCAGTGGAATCACCTGTGGGGGAATTTATATTGG
TGGCTGTTGGATTCTGACTGCTGCACATTGTCTCAGAGCCAGTAAAACTCATCGT
TACCAAATATGGACAACAGTAGTAGACTGGATACACCCCGACCTTAAACGTATA
GTAATTGAATACGTGGATAGAATTATTTTCCATGAAAACTACAATGCAGGCACTT
ACCAAAATGACATCGCTTTGATTGAAATGAAAAAAGACGGAAACAAAAAAGATT
GTGAGCTGCCTCGTTCCATCCCTGCCTGTGTCCCCTGGTCTCCTTACCTATTCCAA
CCTAATGATACATGCATCGTTTCTGGCTGGGGACGAGAAAAAGATAACGAAAGA
GTCTTTTCACTTCAGTGGGGTGAAGTTAAACTAATAAGCAACTGCTCTAAGTTTT
ACGGAAATCGTTTCTATGAAAAAGAAATGGAATGTGCAGGTACATATGATGGTT
CCATCGATGCCTGTAAAGGGGACTCTGGAGGCCCCTTAGTCTGTATGGATGCCAA
CAATGTGACTTATGTCTGGGGTGTTGTGAGTTGGGGGGAAAACTGTGGAAAACC
AGAGTTCCCAGGTGTTTACACCAAAGTGGCCAATTATTTTGACTGGATTAGCTAC
CATGTAGGAAGGCCTTTTATTTCTCAGTACAATGTATAATAAGATATCGATACAT
TGATGAGTTTGGACAAACCACAACTAGAATGCAGTGAAAAAAATGCTTTATTTGT
GAAATTTGTGATGCTATTGCTTTATTTGTAACCATTATAAGCTGCAATAAACAAG
ATATCGTTAACTCGAGGGATCCCACGTGCTGATTTTGTAGGTAACCACGTGCGGA
CCGAGCGGCCGCAGGAACCCCTAGTGATGGAGTTGGCCACTCCCTCTGTGCGCGC
TCGCTCGCTCACTGAGGCCGGGCGACCAAAGGTCGCCCGACGCCCGGGCTTTGC
CCGGGCGGCCTCAGTGAGCGAGCGAGCGCGCAGCTGCCTGCAGGGGCGCCTGAT
GCGGTATTTTCTCCTTACGCATCTGTGCGGTATTTCACACCGCATACGTCAAAGC
AACCATAGTACGCGCCCTGTAGCGGCGCATTAAGCGCGGCGGGTGTGGTGGTTA
CGCGCAGCGTGACCGCTACACTTGCCAGCGCCCTAGCGCCCGCTCCTTTCGCTTT
CTTCCCTTCCTTTCTCGCCACGTTCGCCGGCTTTCCCCGTCAAGCTCTAAATCGGG
GGCTCTGTTTAGGGTTCCGATTTAGTGCTTTACGGCACCTCGACCCCAAAAAACT
TGATTTGGGTGATGGTTCACGTAGTGGGCCATCGCCCTGATAGACGGTTTTTCGC
CCTTTGACGTTGGAGTCCACGTTCTTTAATAGTGGACTCTTGTTCCAAACTGGAA
CAACACTCAACCCTATCTCGGGCTATTCTTTTGATTTATAAGGGATTTTGCCGATT
TCGGCCTATTGGTTAAAAAATGAGCTGATTTAACAAAAATTTAACGCGAATTTTA
ACAAAATATTAACGTTTACAATTTTATGGTGCACTCTCAGTACAATCTGCTCTGAT
GCCGCATAGTTAAGCCAGCCCCGACACCCGCCAACACCCGCTGACGCGCCCTGA
CGGGCTTGTCTGCTCCCGGCATCCGCTTACAGACAAGCTGTGACCGTCTCCGGGA
GCTGCATGTGTCAGAGGTTTTCACCGTCATCACCGAAACGCGCGAGACGAAAGG
GCCTCGTGATACGCCTATTTTTATAGGTTAATGTCATGATAATAATGGTTTCTTAG
ACGTCAGGTGGCACTTTTCGGGGAAATGTGCGCGGAACCCCTATTTGTTTATTTTT
CTAAATACATTCAAATATGTATCCGCTCATGAGACAATAACCCTGATAAATGCTT
CAATAATATTGAAAAAGGAAGAGTATGAGTATTCAACATTTCCGTGTCGCCCTTA
TTCCCTTTTTTGCGGCATTTTGCCTTCCTGTTTTTGCTCACCCAGAAACGCTGGTG
AAAGTAAAAGATGCTGAAGATCAGTTGGGTGCACGAGTGGGTTACATCGAACTG
GATCTCAACAGCGGTAAGATCCTTGAGAGTTTTCGCCCCGAAGAACGTTTTCCAA
TGATGAGCACTTTTAAAGTTCTGCTATGTGGCGCGGTATTATCCCGTATTGACGC
CGGGCAAGAGCAACTCGGTCGCCGCATACACTATTCTCAGAATGACTTGGTTGA
GTACTCACCAGTCACAGAAAAGCATCTTACGGATGGCATGACAGTAAGAGAATT
ATGCAGTGCTGCCATAACCATGAGTGATAACACTGCGGCCAACTTACTTCTGACA
GTAACTCGCCTGATCGTTGGGAACCGGAGCTGAATGAAGCCATACCAAACGAC
GAGCGTGACACCACGATGCCTGTAGCAATGGCAACAACGTTGCGCAAACTATTA
ACTGGCGAACTACTTACTCTAGCTTCCCGGCAACAATTAATAGACTGGATGGAGG
CGGATAAAGTTGCAGGACCACTTCTGCGCTCGGCCCTTCCGGCTGGCTGGTTTAT
TGCTGATAAATCTGGAGCCGGTGAGCGTGGGTCTCGCGGTATCATTGCAGCACTG
GGGCCAGATGGTAAGCCCTCCCGTATCGTAGTTATCTACACGACGGGGAGTCAG
GCAACTATGGATGAACGAAATAGACAGATCGCTGAGATAGGTGCCTCACTGATT
AAGCATTGGTAACTGTCAGACCAAGTTTTACTCATATATACTTTAGATTGATTTAA
AACTTCATTTTTAATTTAAAAGGATCTAGGTGAAGATCCTTTTTGATAATCTCATG
ACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAA
ACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCC
TTCTAGTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACACC
GAACTGAGATACCTACAGCGTGAGCTATGAGAAAGCGCCACGCTTCCCGAAGGG
AGAAAGGCGGACAGGTATCCGGTAAGCGGCAGGGTCGGAACAGGAGAGCGCAC
GAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTAT
GGAAAAACGCCAGCAACGCGGCCTTTTTACGGTTCCTGGCCTTTTGCTGGCCTTT
TGCTCACATGT
SEQ ID NO: 21-CRALBP Promoter
GTTAACGTCCTCTCCCTGCTTGGCCTTAACCAGCCACATTTCTCAACTGACCCCAC
TCACTGCAGAGGTGAAAACTACCATGCCAGGTCCTGCTGGCTGGGGGAGGGGTG
GGCAATAGGCCTGGATTTGCCAGAGCTGCCACTGTAGATGTAGTCATATTTACGA
TTTCCCTTCACCTCTTATAACCCTGGTGGTGGTGGTGGGGGGGGGGGGGTGCTCT
CTCAGCAACCCCACCCCGGGATCTTGAGGAGAAAGAGGGCAGAGAAAAGAGGG
AATGGGACTGGCCCAGATCCCAGCCCCACAGCCGGGCTTCCACATGGCCGAGCA
GGAACTCCAGAGCAGGAGCACACAAAGGAGGGCTTTGATGCGCCTCCAGCCAGG
CCCAGGCCTCTCCCCTCTCCCCTTTCTCTCTGGGTCTTCCTTTGCCCCACTGAGGG
CCTCCTGTGAGCCCGATTTAACGGAAACTGTGGGCGGTGAGAAGTTCCTTATGAC
ACACTAATCCCAACCTGCTGACCGGACCACGCCTCCAGCGGAGGGAACCTCTAG
AGCTCCAGGACATTCAGGTACCAGGTAGCCCCAAGGAGGAGCTGCCGACC
SEQ ID NO: 22-Representative CFI AAV Vector (with CRALBP Promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTGTTAACG
TCCTCTCCCTGCTTGGCCTTAACCAGCCACATTTCTCAACTGACCCCACTCACTGC
AGAGGTGAAAACTACCATGCCAGGTCCTGCTGGCTGGGGGAGGGGTGGGCAATA
GGCCTGGATTTGCCAGAGCTGCCACTGTAGATGTAGTCATATTTACGATTTCCCT
TCACCTCTTATTACCCTGGTGGTGGTGGTGGGGGGGGGGGGGTGCTCTCTCAGCA
ACCCCACCCCGGGATCTTGAGGAGAAAGAGGGCAGAGAAAAGAGGGAATGGGA
CTGGCCCAGATCCCAGCCCCACAGCCGGGCTTCCACATGGCCGAGCAGGAACTC
CAGAGCAGGAGCACACAAAGGAGGGCTTTGATGCGCCTCCAGCCAGGCCCAGGC
CTCTCCCCTCTCCCCTTTCTCTCTGGGTCTTCCTTTGCCCCACTGAGGGCCTCCTGT
GAGCCCGATTTAACGGAAACTGTGGGCGGTGAGAAGTTCCTTATGACACACTAA
TCCCAACCTGCTGACCGGACCACGCCTCCAGCGGAGGGAACCTCTAGAGCTCCA
GGACATTCAGGTACCAGGTAGCCCCAAGGAGGAGCTGCCGACCACCGGTCTCGA
AGGCCTGCAGGCGGCCGCCGCCACCATGAAGCTTCTTCATGTTTTCCTGTTATTTC
TGTGCTTCCACTTAAGGTTTTGCAAGGTCACTTATACATCTCAAGAGGATCTGGT
GGAGAAAAAGTGCTTAGCAAAAAAATATACTCACCTCTCCTGCGATAAAGTCTT
CTGCCAGCCATGGCAGAGATGCATTGAGGGCACCTGTGTTTGTAAACTACCGTAT
CAGTGCCCAAAGAATGGCACTGCAGTGTGTGCAACTAACAGGAGAAGCTTCCCA
ACATACTGTCAACAAAAGAGTTTGGAATGTCTTCATCCAGGGACAAAGTTTTTAA
ATAACGGAACATGCACAGCCGAAGGAAAGTTTAGTGTTTCCTTGAAGCATGGAA
ATACAGATTCAGAGGGAATAGTTGAAGTAAAACTTGTGGACCAAGATAAGACAA
TGTTCATATGCAAAAGCAGCTGGAGCATGAGGGAAGCCAACGTGGCCTGCCTTG
ACCTTGGGTTTCAACAAGGTGCTGATACTCAAAGAAGGTTTAAGTTGTCTGATCT
CTCTATAAATTCCACTGAATGTCTACATGTGCATTGCCGAGGATTAGAGACCAGT
TTGGCTGAATGTACTTTTACTAAGAGAAGAACTATGGGTTACCAGGATTTCGCTG
ATGTGGTTTGTTATACACAGAAAGCAGATTCTCCAATGGATGACTTCTTTCAGTG
TGTGAATGGGAAATACATTTCTCAGATGAAAGCCTGTGATGGTATCAATGATTGT
GGAGACCAAAGTGATGAACTGTGTTGTAAAGCATGCCAAGGCAAAGGCTTCCAT
TGCAAATCGGGTGTTTGCATTCCAAGCCAGTATCAATGCAATGGTGAGGTGGACT
GCATTACAGGGGAAGATGAAGTTGGCTGTGCAGGCTTTGCATCTGTGACTCAAG
AAGAAACAGAAATTTTGACTGCTGACATGGATGCAGAAAGAAGACGGATAAAAT
CATTATTACCTAAACTATCTTGTGGAGTTAAAAACAGAATGCACATTCGAAGGAA
ACGAATTGTGGGAGGAAAGCGAGCACAACTGGGAGACCTCCCATGGCAGGTGGC
AATTAAGGATGCCAGTGGAATCACCTGTGGGGGAATTTATATTGGTGGCTGTTGG
ATTCTGACTGCTGCACATTGTCTCAGAGCCAGTAAAACTCATCGTTACCAAATAT
GGACAACAGTAGTAGACTGGATACACCCCGACCTTAAACGTATAGTAATTGAAT
ACGTGGATAGAATTATTTTCCATGAAAACTACAATGCAGGCACTTACCAAAATG
ACATCGCTTTGATTGAAATGAAAAAAGACGGAAACAAAAAAGATTGTGAGCTGC
CTCGTTCCATCCCTGCCTGTGTCCCCTGGTCTCCTTACCTATTCCAACCTAATGAT
ACATGCATCGTTTCTGGCTGGGGACGAGAAAAAGATAACGAAAGAGTCTTTTCA
CTTCAGTGGGGTGAAGTTAAACTAATAAGCAACTGCTCTAAGTTTTACGGAAATC
GTTTCTATGAAAAAGAAATGGAATGTGCAGGTACATATGATGGTTCCATCGATGC
CTGTAAAGGGGACTCTGGAGGCCCCTTAGTCTGTATGGATGCCAACAATGTGACT
TATGTCTGGGGTGTTGTGAGTTGGGGGGAAAACTGTGGAAAACCAGAGTTCCCA
GGTGTTTACACCAAAGTGGCCAATTATTTTGACTGGATTAGCTACCATGTAGGAA
GGCCTTTTATTTCTCAGTACAATGTATAATAAGATATCGATACATTGATGAGTTTG
GACAAACCACAACTAGAATGCAGTGAAAAAAATGCTTTATTTGTGAAATTTGTG
ATGCTATTGCTTTATTTGTAACCATTATAAGCTGCAATAAACAAGATATCGTTAA
CTCGAGGGATCCCACGTGCTGATTTTGTAGGTAACCACGTGCGGACCGAGCGGC
CGCAGGAACCCCTAGTGATGGAGTTGGCCACTCCCTCTCTGCGCGCTCGCTCGCT
CACTGAGGCCGGGCGACCAAAGGTCGCCCGACGCCCGGGCTTTGCCCGGGCGGC
CTCAGTGAGCGAGCGAGCGCGCAGCTGCCTGCAGGGGCGCCTGATGCGGTATTT
TCTCCTTACGCATCTGTGCGGTATTTCACACCGCATACGTCAAAGCAACCATAGT
ACGCGCCCTGTAGCGGCGCATTAAGCGCGGCGGGTGTGGTGGTTACGCGCAGCG
TGACCGCTACACTTGCCAGCGCCCTAGCGCCCGCTCCTTTCGCTTTCTTCCCTTCC
TTTCTCGCCACGTTCGCCGGCTTTCCCCGTCAAGCTCTAAATCGGGGGCTCCCTTT
AGGGTTCCGATTTAGTGCTTTACGGCACCTCGACCCCAAAAAACTTGATTTGGGT
GATGGTTCACGTAGTGGGCCATCGCCCTGATAGACGGTTTTTCGCCCTTTGACGT
TGGAGTCCACGTTCTTTAATAGTGGACTCTTGTTCCAAACTGGAACAACACTCAA
CCCTATCTCGGGCTATTCTTTTGATTTATAAGGGATTTTGCCGATTTCGGCCTATT
GGTTAAAAAATGAGCTGATTTAACAAAAATTTAACGCGAATTTTAACAAAATATT
AACGTTTACAATTTTATGGTGCACTCTCAGTACAATCTGCTCTGATGCCGCATAG
TTAAGCCAGCCCCGACACCCGCCAACACCCGCTGACGCGCCCTGACGGGCTTGTC
TGCTCCCGGCATCCGCTTACAGACAAGCTGTGACCGTCTCCGGGAGCTGCATGTG
TCAGAGGTTTTCACCGTCATCACCGAAACGCGCGAGACGAAAGGGCCTCGTGAT
ACGCCTATTTTTATAGGTTAATGTCATGATAATAATGGTTTCTTAGACGTCAGGTG
GCACTTTTCGGGGAAATGTGCGCGGAACCCCTATTTGTTTATTTTTCTAAATACAT
TCAAATATGTATCCGCTCATGAGACAATAACCCTGATAAATGCTTCAATAATATT
GAAAAAGGAAGAGTATGAGTATTCAACATTTCCGTGTCGCCCTTATTCCCTTTTTT
GCGGCATTTTGCCTTCCTGTTTTTGCTCACCCAGAAACGCTGGTGAAAGTAAAAG
ATGCTGAAGATCAGTTGGGTGCACGAGTGGGTTACATCGAACTGGATCTCAACA
GCGGTAAGATCCTTGAGAGTTTTCGCCCCGAAGAACGTTTTCCAATGATGAGCAC
TTTTAAAGTTCTGCTATGTGGCGCGGTATTATCCCGTATTGACGCCGGGCAAGAG
CAACTCGGTCGCCGCATACACTATTCTCAGAATGACTTGGTTGAGTACTCACCAG
TCACAGAAAAGCATCTTACGGATGGCATGACAGTAAGAGAATTATGCAGTGCTG
CCATAACCATGAGTGATAACACTGCGGCCAACTTACTTCTGACAACGATCGGAG
GACCGAAGGAGCTAACCGCTTTTTTGCACAACATGGGGGATCATGTAACTCGCCT
TGATCGTTGGGAACCGGAGCTGAATGAAGCCATACCAAACGACGAGCGTGACAC
CACGATGCCTGTAGCAATGGCAACAACGTTGCGCAAACTATTAACTGGCGAACT
ACTTACTCTAGCTTCCCGGCAACAATTAATAGACTGGATGGAGGCGGATAAAGTT
GCAGGACCACTTCTGCGCTCGGCCCTTCCGGCTGGCTGGTTTATTGCTGATAAAT
CTGGAGCCGGTGAGCGTGGGTCTCGCGGTATCATTGCAGCACTGGGGCCAGATG
GTAAGCCCTCCCGTATCGTAGTTATCTACACGACGGGGAGTCAGGCAACTATGG
ATGAACGAAATAGACAGATCGCTGAGATAGGTGCCTCACTGATTAAGCATTGGT
AACTGTCAGACCAAGTTTACTCATATATACTTTAGATTGATTTAAAACTTCATTTT
TAATTTAAAAGGATCTAGGTGAAGATCCTTTTTGATAATCTCATGACCAAAATCC
CTTAACGTGAGTTTTCGTTCCACTGAGCGTCAGACCCCGTAGAAAAGATCAAAGG
ATCTTCTTGAGATCCTTTTTTTCTGCGCGTAATCTGCTGCTTGCAAACAAAAAAAC
CACCGCTACCAGCGGTGGTTTGTTTGCCGGATCAAGAGCTACCAACTCTTTTTCC
GAAGGTAACTGGCTTCAGCAGAGCGCAGATACCAAATACTGTCCTTCTAGTGTA
GCCGTAGTTAGGCCACCACTTCAAGAACTCTGTAGCACCGCCTACATACCTCGCT
CTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGG
GGGGTTCGTGCACACAGCCCAGCTTGGAGCGAACGACCTACACCGAACTGAGAT
ACCTACAGCGTGAGCTATGAGAAAGCGCCACGCTTCCCGAAGGGAGAAAGGCGG
ACAGGTATCCGGTAAGCGGCAGGGTCGGAACAGGAGAGCGCACGAGGGAGCTT
CCAGGGGGAAACGCCTGGTATCTTTATAGTCCTGTCGGGTTTCGCCACCTCTGAC
TTGAGCGTCGATTTTTGTGATGCTCGTCAGGGGGGCGGAGCCTATGGAAAAACG
CCAGCAACGCGGCCTTTTTACGGTTCCTGGCCTTTTGCTGGCCTTTTGCTCACATG
T
SEQ ID NO: 23-EF1a Promoter
GGGCAGAGCGCACATCGCCCACAGTCCCCGAGAAGTTGGGGGGAGGGGTCGGC
AATTGAACCGGTGCCTAGAGAAGGTGGCGCGGGGTAAACTGGGAAAGTGATGTC
GTGTACTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAG
SEQ ID NO: 24-Representative CFI AAV Vector (with EF1a Promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTGTTAACG
GGCAGAGCGCACATCGCCCACAGTCCCCGAGAAGTTGGGGGGAGGGGTCGGCA
ATTGAACCGGTGCCTAGAGAAGGTGGCGCGGGGTAAACTGGGAAAGTGATGTCG
TGTACTGGCTCCGCCTTTTTCCCGAGGGTGGGGGAGAACCGTATATAAGTGCAGT
AGTCGCCGTGAACGTTCTTTTTCGCAACGGGTTTGCCGCCAGAACACAGACCGGT
CTCGAAGGCCTGCAGGCGGCCGCCGCCACCATGAAGCTTCTTCATGTTTTCCTGT
TATTTCTGTGCTTCCACTTAAGGTTTTGCAAGGTCACTTATACATCTCAAGAGGAT
CTGGTGGAGAAAAAGTGCTTAGCAAAAAAATATACTCACCTCTCCTGCGATAAA
GTCTTCTGCCAGCCATGGCAGAGATGCATTGAGGGCACCTGTGTTTGTAAACTAC
CGTATCAGTGCCCAAAGAATGGCACTGCAGTGTGTGCAACTAACAGGAGAAGCT
TCCCAACATACTGTCAACAAAAGAGTTTGGAATGTCTTCATCCAGGGACAAAGTT
TTTAAATAACGGAACATGCACAGCCGAAGGAAAGTTTAGTGTTTCCTTGAAGCAT
GGAAATACAGATTCAGAGGGAATAGTTGAAGTAAAACTTGTGGACCAAGATAAG
ACAATGTTCATATGCAAAAGCAGCTGGAGCATGAGGGAAGCCAACGTGGCCTGC
CTTGACCTTGGGTTTCAACAAGGTGCTGATACTCAAAGAAGGTTTAAGTTGTCTG
ATCTCTCTATAAATTCCACTGAATGTCTACATGTGCATTGCCGAGGATTAGAGAC
CAGTTTGGCTGAATGTACTTTTACTAAGAGAAGAACTATGGGTTACCAGGATTTC
GCTGATGTGGTTTGTTATACACAGAAAGCAGATTCTCCAATGGATGACTTCTTTC
AGTGTGTGAATGGGAAATACATTTCTCAGATGAAAGCCTGTGATGGTATCAATGA
TTGTGGAGACCAAAGTGATGAACTGTGTTGTAAAGCATGCCAAGGCAAAGGCTT
CCATTGCAAATCGGGTGTTTGCATTCCAAGCCAGTATCAATGCAATGGTGAGGTG
GACTGCATTACAGGGGAAGATGAAGTTGGCTGTGCAGGCTTTGCATCTGTGACTC
AAGAAGAAACAGAAATTTTGACTGCTGACATGGATGCAGAAAGAAGACGGATA
AAATCATTATTACCTAAACTATCTTGTGGAGTTAAAAACAGAATGCACATTCGAA
GGAAACGAATTGTGGGAGGAAAGCGAGCACAACTGGGAGACCTCCCATGGCAG
GTGGCAATTAAGGATGCCAGTGGAATCACCTGTGGGGGAATTTATATTGGTGGCT
GTTGGATTCTGACTGCTGCACATTGTCTCAGAGCCAGTAAAACTCATCGTTACCA
AATATGGACAACAGTAGTAGACTGGATACACCCCGACCTTAAACGTATAGTAAT
TGAATACGTGGATAGAATTATTTTCCATGAAAACTACAATGCAGGCACTTACCAA
AATGACATCGCTTTGATTGAAATGAAAAAAGACGGAAACAAAAAAGATTGTGAG
CTGCCTCGTTCCATCCCTGCCTGTGTCCCCTGGTCTCCTTACCTATTCCAACCTAA
TGATACATGCATCGTTTCTGGCTGGGGACGAGAAAAAGATAACGAAAGAGTCTT
TTCACTTCAGTGGGGTCAAGTTAAACTAATAAGCAACTGCTCTAAGTTTTACGGA
AATCGTTTCTATGAAAAAGAAATGGAATGTGCAGGTACATATGATGGTTCCATCG
ATGCCTCTAAAGGGGACTCTGGAGGCCCCTTAGTCTGTATGGATGCCAACAATGT
GACTTATGTCTGGGGTGTTGTGAGTTGGGGGGAAAACTGTGGAAAACCAGAGTT
CCCAGGTGTTTACACCAAAGTGGCCAATTATTTTGACTGGATTAGCTACCATGTA
GGAAGGCCTTTTATTTCTCAGTACAATGTATAATAAGATATCGATACATTGATGA
GTTTGGACAAACCACAACTAGAATGCAGTGAAAAAAATGCTTTATTTGTGAAATT
TGTGATGCTATTGCTTTATTTGTAACCATTATAAGCTGCAATAAACAAGATATCG
TTAACTCGAGGGATCCCACGTGCTGATTTTGTAGGTAACCACGTGCGGACCGAGC
GGCCGCAGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCCCGGGC
GGCCTCAGTGAGCGAGCGAGCGCGCAGCTGCCTGCAGGGGCGCCTGATGCGGTA
TTTTCTCCTTACGCATCTGTGCGGTATTTCACACCGCATACGTCAAAGCAACCAT
AGTACGCGCCCTGTAGCGGCGCATTAAGCGCGGCGGGTGTGGTGGTTACGCGCA
GCGTGACCGCTACACTTGCCAGCGCCCTAGCGCCCGCTCCTTTCGCTTTCTTCCCT
TCCTTTCTCGCCACGTTCGGCGGCTTTCCCCGTCAAGCTCTAAATCGGGGGCTCCC
TTTAGGGTTCCGATTTAGTGCTTTACGGCACCTCGACCCCAAAAAACTTGATTTG
GGTGATGGTTCACGTAGTGGGCCATCGCCCTGATAGACGGTTTTTCGCCCTTTGA
CGTTGGAGTCCACGTTCTTTAATAGTGGACTCTTGTTCCAAACTGGAACAACACT
CAACCCTATCTCGGGCTATTCTTTTGATTTATAAGGGATTTTGCCGATTTCGGCCT
ATTGGTTAAAAAATGAGCTGATTTAACAAAAATTTAACGCGAATTTTAACAAAAT
ATTAACGTTTACAATTTTATGGTGCACTCTCAGTACAATCTGCTCTGATGCCGCAT
AGTTAAGCCAGCCCCGACACCCGCCAACACCCGCTGACGCGCCCTGACGGGCTT
GTCTGCTCCCGGCATCCGCTTACAGACAAGCTGTGACCGTCTCCGGGAGCTGCAT
GTGTCAGAGGTTTTCACCGTCATCACCGAAACGCGCGAGACGAAAGGGCCTCGT
GATACGCCTATTTTTATAGGTTAATGTCATGATAATAATGGTTTCTTAGACGTCAG
GTGGCACTTTTCGGGGAAATGTGCGCGGAACCCCTATTTGTTTATTTTTCTAAATA
CATTCAAATATGTATCCGCTCATGAGACAATAACCCTGATAAATGCTTCAATAAT
ATTGAAAAAGGAAGAGTATGAGTATTCAACATTTCCGTGTCGCCCTTATTCCCTT
TTTTGCGGCATTTTGCCTTCCTGTTTTTGCTCACCCAGAAACGCTGGTGAAAGTAA
AAGATGCTGAAGATCAGTTGGGTGCACGAGTGGGTTACATCGAACTGGATCTCA
ACAGCGGTAAGATCCTTGAGAGTTTTCGCCCCGAAGAACGTTTTCCAATGATGAG
CACTTTTAAAGTTCTGCTATGTGGCGCGGTATTATCCCGTATTGACGCCGGGCAA
GAGCAACTCGGTCGCCGCATACACTATTCTCAGAATGACTTGGTTGAGTACTCAC
CAGTCACAGAAAAGCATCTTACGGATGGCATGACAGTAAGAGAATTATGCAGTG
CTGCCATAACCATGAGTGATAACACTGCGGCCAACTTACTTCTGACAACGATCGG
AGGACCGAAGGAGCTAACCGCTTTTTTGCACAACATGGGGGATCATGTAACTCG
CCTTGATCGTTGGGAACCGGAGCTGAATGAAGCCATACCAAACGACGAGCGTGA
CACCACGATGCCTGTAGCAATGGCAACAACGTTGCGCAAACTATTAACTGGCGA
ACTACTTACTCTAGCTTCCCGGCAACAATTAATAGACTGGATGGAGGCGGATAA
AGTTGCAGGACCACTTCTGCGCTCGGCCCTTCCGGCTGGCTGGTTTATTGCTGAT
AAATCTGGAGCCGGTGAGCGTGGGTCTCGCGGTATCATTGCAGCACTGGGGCCA
GATGGTAAGCCCTCCCGTATCGTAGTTATCTACACGACGGGGAGTCAGGCAACT
ATGGATGAACGAAATAGACAGATCGCTGAGATAGGTGCCTCACTGATTAAGCAT
TGGTAACTGTCAGACCAAGTTTACTCATATATACTTTAGATTGATTTAAAACTTCA
TTTTTAATTTAAAAGGATCTAGGTGAAGATCCTTTTTGATAATCTCATGACCAAA
ATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAA
AAACCACCGCTACCAGCGGTGGTTTGTTTGCCGGATCAAGAGCTACCAACTCTTT
TTCCGAAGGTAACTGGCTTCAGCAGAGCGCAGATACCAAATACTGTCCTTCTAGT
GTAGCCGTAGTTAGGCCACCACTTCAAGAACTCTGTAGCACCGCCTACATACCTC
GCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAA
CGGGGGGTTCGTGCACACAGCCCAGCTTGGAGCGAACGACCTACACCGAACTGA
GATACCTACAGCGTGAGCTATGAGAAAGCGCCACGCTTCCCGAAGGGAGAAAGG
CGGACAGGTATCCGGTAAGCGGCAGGGTCGGAACAGGAGAGCGCACGAGGGAG
CTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAA
ACGCCAGCAACGCGGCCTTTTTACGGTTCCTGGCCTTTTGCTGGCCTTTTGCTCAC
ATGT
SEQ ID NO: 25-URPE65 Promoter
GTTAACTATATTTATTGAAGTTTAATATTGTGTTTGTGATACAGAAGTATTTGCTT
TAATTCTAAATAAAAATTTTATGCTTTTATTGCTGGTTTAAGAAGATTTGGATTAT
CCTTGTACTTTGAGGAGAAGTTTCTTATTTGAAATATTTTGGAAACAGGTCTTTTA
ATGTGGAAAGATAGATATTAATCTCCTCTTCTATTACTCTCCAAGATCCAACAAA
AGTGATTATACCCCCCAAAATATGATGGTAGTATCTTATACTACCATCATTTTATA
GGCATAGGGCTCTTAGCTGCAAATAATGGAACTAACTCTAATAAAGCAGAACGC
AAATATTGTAAATATTAGAGAGCTAACAATCTCTGGGATGGCTAAAGGATGGAG
CTTGGAGGCTACCCAGCCAGTAACAATATTCCGGGCTCCACTGTTGAATGGAGAC
ACTACAACTGCCTTGGATGGGCAGAGATATTATGGATCCTAAGCCCCAGGTGCT
ACCATTAGGACTTCTACCACTGTCCCTAACGGGTGGAGCCCATCACATGCCTATG
CCCTCACTGTAAGGAAATGAAGCTACTGTTGTATATCTTGGGAAGCACTTGGATT
AATTGTTATACAGTTTTGTTGAAGAAGACCCCTAGGGTAAGTAGCCATAACTGCA
CACTAAATTTAAAATTGTTAATGAGTTTCTCAAAAAAAATGTTAAGGTTGTTAGC
TGGTATAGTATATATCTTGCCTGTTTTCCAAGGACTTCTTTGGGCAGTACCTTGTC
TGTGCTGGCAAGCAACTGAGACTTAATGAAAGAGTATTGGAGATATGAATGAAT
TGATGCTGTATACTCTCAGAGTGCCAAACATATACCAATGGACAAGAAGGTGAG
GCAGAGAGCAGACAGGCATTAGTGACAAGCAAAGATATGCAGAATTTCATTCTC
AGCAAATCAAAAGTCCTCAACCTGGTTGGAAGAATATTGGCACTGAATGGTATC
AATAAGGTTGCTAGAGAGGGTTAGAGGTGCACAATGTTGCTTCCATAACATTITAT
ACTTCTCCAATCTTAGCACTAATCAAACATGGTTGAATACTTTGTTTACTATAACT
CTTACAGAGTTATAAGATCTGTGAAGACAGGGACAGGGACAATACCCATCTCTG
TCTGGTTCATAGGTGGTATGTAATAGATATTTTTAAAAATAAGTGAGTTAATGAA
TGAGGGTGAGAATGAAGGCACAGAGGTATTAGGGGGAGGTGGGCCCCAGAGAA
TGGTGCCAAGGTCCAGTGGGGTGACTGGGATCAGCTCAGGCCTGACGCTGGCCA
CTCCCACCTAGCTCCTTTCTTTCTAATCTGTTCTCATTCTCCTTGGGAAGGATTGA
GGTCTCTGGAAAACAGCCAAACAACTGTTATGGGAACAGCAAGCCCAAATAAAG
CCAAGCATCAGGGGGATCTGAGAGCTGAAAGCAACTTTCTGTTCCCCCTCCCTCAG
CTGAAGGGGTGGGGAAGGGCTCCCAAAGCCATAACTCCTTTTAAGGGATTTAGA
AGGCATAAAAAGGCCCCTGGCTGAGAACTTCCTTCTTCATTCTGCAGTTGG
SEQ ID NO: 26-Representative CFI AAV Vector (with HRPE65 Promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTGTTAACT
ATATTTATTGAAGHTAATATTGTGTTTGTGATACAGAAGTATTTGCTTTAATTCT
AAATAAAAATTTTATGCTTTTATTGCTGGTTTAAGAAGATTTGGATTATCCTTGTA
CTTTGAGGAGAAGTTTCTTATTTGAAATATTTTGGAAACAGGTCTTTTAATGTGG
AAAGATAGATATTAATCTCCTCTTCTATTACTCTCCAAGATCCAACAAAAGTGAT
TATACCCCCCAAAATATGATGGTAGTATCTTATACTACCATCATTTTATAGGCAT
AGGGCTCTTAGCTGCAAATAATGGAACTAACTCTAATAAAGCAGAACGCAAATA
TTGTAAATATTAGAGAGCTAACAATCTCTGGGATGGCTAAAGGATGGAGCTTGG
AGGCTACCCAGCCAGTAACAATATTCCGGGCTCCACTGTTGAATGGAGACACTA
CAACTGCCTTGGATGGGCAGAGATATTATGGATGCTAAGCCCCAGGTGCTACCAT
TAGGACTTCTACCACTGTCCCTAACGGGTGGAGCCCATCACATGCCTATGCCCTC
ACTGTAAGGAAATGAAGCTACTGTTGTATATCTTGGGAAGCACTTGGATTAATTG
TTATACAGTTTTGTTGAAGAAGACCCCTAGGGTAAGTAGCCATAACTGCACACTA
AATTTAAAATTGTTAATGAGTTTCTCAAAAAAAATGTTAAGGTTGTTAGCTGGTA
TAGTATATATCTTGCCTGTTTTCCAAGGACTTCTTTGGGCAGTACCTTGTCTGTGC
TGGCAAGCAACTGAGACTTAATGAAAGAGTATTGGAGATATGAATGAATTGATG
CTGTATACTCTCAGAGTGCCAAACATATACCAATGGACAAGAAGGTGAGGCAGA
GAGCAGACAGGCATTAGTGACAAGCAAAGATATGCAGAATTTCATTCTCAGCAA
ATCAAAAGTCCTCAACCTGGTTGGAAGAATATTGGCACTGAATGGTATCAATAA
GGTTGCTAGAGAGGGTTAGAGGTGCACAATGTGCTTCCATAACATTTTATACTTC
TCCAATCTTAGCACTAATCAAACATGGTTGAATACTTTGTTTACTATAACTCTTAC
AGAGTTATAAGATCTGTGAAGACAGGGACAGGGACAATACCCATCTCTGTCTGG
TTCATAGGTGGTATGTAATAGATATTTTTAAAAATAAGTGAGTTAATGAATGAGG
GTGAGAATGAAGGCACAGAGGTATTAGGGGGAGGTGGGCCCCAGAGAATGGTG
CCAAGGTCCAGTGGGGTGACTGGGATCAGCTCAGGCCTGACGCTGGCCACTCCC
ACCTAGCTCCTTTCTTTCTAATCTGTTCTCATTCTCCTTGGGAAGGATTGAGGTCT
CTGGAAAACAGCCAAACAACTGTTATGGGAACAGCAAGGCCAAATAAAGCCAA
GCATCAGGGGGATCTGAGAGCTGAAAGCAACTTCTGTTCCCCCTCCCTCAGCTGA
AGGGGTGGGGAAGGGCTCCCAAAGCCATAACTCCTTTTAAGGGATTTAGAAGGC
ATAAAAAGGCCCCTGGCTGAGAACTTCCTTCTTCATTCTGCAGTTGGACCGGTCT
CGAAGGCCTGCAGGCGGCCGCCGCCACCATGAAGCTTCTTCATGTTTTCCTGTTA
TTTCTGTGCTTCCACTTAAGGTTTTGCAAGGTCACTTATACATCTCAAGAGGATCT
GGTGGAGAAAAAGTGCTTAGCAAAAAAATATACTCACCTCTCCTGCGATAAAGT
CTTCTGCCAGCCATGGCAGAGATGCATTGAGGGCACCTGTGTTTGTAAACTACCG
TATCAGTGCCCAAAGAATGGCACTGCAGTGTGTGCAACTAACAGGAGAAGCTTC
CCAACATACTGTCAACAAAAGAGTTTGGAATGTCTTCATCCAGGGACAAAGTTTT
TAAATAACGGAACATGCACAGCCGAAGGAAAGTTTAGTGTTTCCTTGAAGCATG
GAAATACAGATTCAGAGGGAATAGTTGAAGTAAAACTTGTGGACCAAGATAAGA
CAATGTTCATATGCAAAAGCAGCTGGAGCATGAGGGAAGCCAACGTGGCCTGCC
TTGACCTTGGGTTTCAACAAGGTGCTGATACTCAAAGAAGGTTTAAGTTGTCTGA
TCTCTCTATAAATTCCACTGAATGTCTACATGTGCATTGCCGAGGATTAGAGACC
AGTTTGGCTGAATGTACTTTTACTAAGAGAAGAACTATGGGTTACCAGGATTTCG
CTGATGTGGTTTGTTATACACAGAAAGCAGATTCTCCAATGGATGACTTCTTTCA
GTGTGTGAATGGGAAATACATTTCTCAGATGAAAGCCTGTGATGGTATCAATGAT
TGTGGAGACCAAAGTGATGAACTGTGTTGTAAAGCATGCCAAGGCAAAGGCTTC
CATTGCAAATCGGGTGTTTGCATTCCAAGCCAGTATCAATGCAATGGTGAGGTGG
ACTGCATTACAGGGGAAGATGAAGTTGGCTGTGCAGGCTTTGCATCTGTGACTCA
AGAAGAAACAGAAATTTTGACTGCTGACATGGATGCAGAAAGAAGACGGATAA
AATCATTATTACCTAAACTATCTTGTGGAGTTAAAAACAGAATGCACATTCGAAG
GAAACGAATTGTGGGAGGAAAGCGAGCACAACTGGGAGACCTCCCATGGCAGG
TGGCAATTAAGGATGCCAGTGGAATCACCTGTGGGGGAATTTATATTGGTGGCTG
TTGGATTCTGACTGCTGCACATTGTCTCAGAGCCAGTAAAACTCATCGTTACCAA
ATATGGACAACAGTAGTAGACTGGATACACCCCGACCTTAAACGTATAGTAATT
GAATACGTGGATAGAATTATTTTCCATGAAAACTACAATGCAGGCACTTACCAA
AATGACATCGCTTTGATTGAAATGAAAAAAGACGGAAACAAAAAAGATTGTGAG
CTGCCTCGTTCCATCCCTGCCTGTGTCCCCTGGTCTCCTTACCTATTCCAACCTAA
TGATACATGCATCGTTTCTGGCTGGGGACGAGAAAAAGATAACGAAAGAGTCTT
TTCACTTCAGTCGGGTGAAGTTAAACTAATAAGCAACTGCTCTAAGTTTTACGGA
AATCGTTTCTATGAAAAAGAAATGGAATGTGCAGGTACATATGATGGTTCCATCG
ATGCCTGTAAAGGGGACTCTGGAGGCCCCTTAGTCTGTATGGATGCCAACAATGT
GACTTATGTCTGGGGTGTTGTGAGTTGGGGGGAAAACTGTGGAAAACCAGAGTT
CCCAGGTGTTTACACCAAAGTGGCCAATTATTTTGACTGGATTAGCTACCATGTA
GGAAGGCCTTTTATTTCTCAGTACAATGTATAATAAGATATCGATACATTGATGA
GTTTGGACAAACCACAACTAGAATGCAGTGAAAAAAATGCTTTATTTGTGAAATT
TGTGATGCTATTGCTTTATTTGTAACCATTATAAGCTGCAATAAACAAGATATCG
TTAACTCGAGGGATCCCACGTGCTGATTTTGTAGGTAACCACGTGCGGACCGAGC
GGCCGCAGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCCCGGGC
GGCCTCAGTGAGCGAGCGAGCGCGCAGCTGCCTGCAGGGGCGCCTGATGCGGTA
TTTTCTCCTTACGCATCTGTGCGGTATTTCACACCGCATACGTCAAAGCAACCAT
AGTACGCGCCCTGTAGCGGCGCATTAAGCGCGGCGGGTGTGGTGGTTACGCGCA
GCGTGACCGCTACACTTGCCAGCGCCCTAGCGCCCGCTCCTTTCGCTTTCTTCCCT
TCCTTTCTCGCCACGTTCGCCGGCTTTCCCCGTCAAGCTCTAAATCGGGGGCTCCC
TTTAGGGTTCCGATTTAGTGCTTTACGGCACCTCGACCCCAAAAAACTTGATTTG
GGTGATGGTTCACGTAGTGGGCCATCGCCCTGATAGACGGTTTTTCGCCCTTTGA
CGTTGGAGTCCACGTTCTTTAATAGTGGACTCTTGTTCCAAACTGGAACAACACT
CAACCCTATCTCGGGCTATTCTTTTGATTTATAAGGGATTTTGCCGATTTCGGCCT
ATTGGTTAAAAAATGAGCTGATTTAACAAAAATTTAACGCGAATTTTAACAAAAT
ATTAACGTTTACAATTTTATGGTGCACTCTCAGTACAATCTGCTCTGATGCCGCAT
AGTTAAGCCAGCCCCGACACCCGCCAACACCCGCTGACGCGCCCTGACGGGCTT
GTCTGCTCCCGGCATCCGCTTACAGACAAGCTGTGACCGTCTCCGGGAGCTGCAT
GTGTCAGAGGTTTTCACCGTCATCACCGAAACGCGCGAGACGAAAGGGCCTCGT
GATACGCCTATTTTTATAGGTTAATGTCATGATAATAATGGTTTCTTAGACGTCAG
GTGGCACTTTTCGGGGAAATGTGCGCGGAACCCCTATTTGTTTATTTTTCTAAATA
CATTCAAATATGTATCCGCTCATGAGACAATAACCCTGATAAATGCTTCAATAAT
ATTGAAAAAGGAAGAGTATGAGTATTCAACATTTCCGTGTCGCCCTTATTCCCTT
TTTTGCGGCATTTTGCCTTCCTGTTTTTGCTCACCCAGAAACGCTCGTGAAAGTAA
AAGATGCTGAAGATCAGTTGGGTGCACGAGTGGGTTACATCGAACTGGATCTCA
ACAGCGGTAAGATCCTTGAGAGTTTTCGCCCCGAAGAACGTTTTCCAATGATGAG
CACTTTTAAAGTTCTGCTATGTGGCGCGGTATTATCCCGTATTGACGCCGGGCAA
GAGCAACTCGGTCGCCGCATACACTATTCTCAGAATGACTTGGTTGAGTACTCAC
CAGTCACAGAAAAGCATCTTACGGATGGCATGACAGTAAGAGAATTATGCAGTG
CTGCCATAACCATGAGTGATAACACTGCGGCCAACTTACTTCTGACAACGATCGG
AGGACCGAAGGAGCTAACCGCTTTTTTGCACAACATGGGGGATCATGTAACTCG
CCTTGATCGTTGGGAACCGGAGCTGAATGAAGCCATACCAAACGACGAGCGTGA
CACCACGATGCCTGTAGCAATGGCAACAACGTTGCGCAAACTATTAACTGGCGA
ACTACTTACTCTAGCTTCCCGGCAACAATTAATAGACTGGATGGAGGCGGATAA
AGTTGCAGGACCACTTCTGCGCTCGGCCCTTCCGGCTGGCTGGTTTATTGCTGAT
AAATCTGGAGCCGGTGAGCGTGGGTCTCGCGGTATCATTGCAGCACTGGGGCCA
GATGGTAAGCCCTCCCGTATCGTAGTTATCTACACGACGGGGAGTCAGGCAACT
ATGGATGAACGAAATAGACAGATCGCTGAGATAGGTGCCTCACTGATTAAGCAT
TGGTAACTGTCAGACCAAGTTTACTCATATATACTTTAGATTGATTTAAAACTTCA
TTTTTAATTTAAAAGGATCTAGGTGAAGATCCTTTTTGATAATCTCATGACCAAA
ATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAA
AAACCACCGCTACCAGCGGTGGTTTGTTTGCCGGATCAAGAGCTACCAACTCTTT
TTCCGAAGGTAACTGGCTTCAGCAGAGCGCAGATACCAAATACTGTCCTTCTAGT
GTAGCCGTAGTTAGGCCACCACTTCAAGAACTCTGTAGCACCGCCTACATACCTC
GCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAA
CGGGGGGTTCGTGCACACAGCCCAGCTTGGAGCGAACGACCTACACCGAACTGA
GATACCTACAGCGTGAGCTATGAGAAAGCGCCACGCTTCCCGAAGGGAGAAAGG
CGGACAGGTATCCGGTAAGCGGCAGGGTCGGAACAGGAGAGCGCACGAGGGAG
CTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAA
ACGCCAGCAACGCGGCCTTTTTACGGTTCCTGGCCTTTTGCTGGCCTTTTGCTCAC
ATGT
SEQ ID NO: 27-Phosphoenolpyruvate Carboxykinase 1 Promoter
GTTAACAGCCCCCAGTTAGGTTAGGCATTTCCAATCTTTGCCAATAAGCCACATA
TTTGCCCAAGTTAGGGTGCATCCTTCCCATGAACTTTGACTGTGACCTTTGACTAT
GGGGTGACATCTTATAGCTGTGGTGTTTTGCCAACCAGCAGCTCTTGGTACACAA
AATGTGCTGCTAGCAGGTGCCCCGGCCAACCTTGTCCTTGACCCACCTGCCTGTT
AAGAAAAGGGTGTTGTGTTTTGCAACAGCAGTAAAATGGGTCAAGGTTTAGTCA
GTTGGAAGTTGTGTCAAAACTCACTATGGTTGGTTGAGGGCTCGAAGTCTCCCAG
CATTCATTAACAACTATCTGTTCAATGATTATCTCCCTGGGGCGTGTTGCAGTGA
GTTGGCCCAAAGCATAACTGACCCTGGCCGTGATCCAGAGACCTGCCCCCTGAC
GTCAGTGGCGAGCCTCCCTGGGTGCAGCTGAGGGGCAGGGCTATTCTTTTCCACA
GT
SEQ ID NO: 28-Representative CFI AAV Vector (with Phosphoenolpyruvate
Carboxykinase 1 Promoter)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTGCGGCCGCACGCGTGTTAACA
GCCCCCAGTTAGGTTAGGCATTTCCAATCTTTGCCAATAAGCCACATATTTGCCC
AAGTTAGGGTGCATCCTTCCCATGAACTTTGACTGTGACCTTTGACTATGGGGTG
ACATCTTATAGCTGTGGTGTTTTGCCAACCAGCAGCTCTTGGTACACAAAATGTG
CTGCTAGCAGGTGCCCCGGCCAACCTTGTCCTTGACCCACCTGCCTGTTAAGAAA
AGGGTGTTGTGTTTTGCAACAGCAGTAAAATGGGTCAAGGTTTAGTCAGTTGGAA
GTTGTGTCAAAACTCACTATGGTTGGTTGAGGGCTCGAAGTCTCCCAGCATTCAT
TAACAACTATCTGTTCAATGATTATCTCCCTGGGGCGTGTTGCAGTGAGTTGGCC
CAAAGCATAACTGACCCTGGCCGTGATCCAGAGACCTGCCCCCTGACGTCAGTG
GCGAGCCTCGCTGGGTGCAGCTGAGGGGCAGGGCTATTCTTTTCCACAGTACCGG
TCTCGAAGGCCTGCAGGCGGCCGCCGCCACCATGAAGCTTCTTCATGTTTTCCTG
TTATTTCTGTGCTTCCACTTAAGGTTTTGCAAGGTCACTTATACATCTCAAGAGGA
TCTGGTGGAGAAAAAGTGCTTAGCAAAAAAATATACTCACCTCTCCTGCGATAA
AGTCTTCTGCCAGCCATGGCAGAGATGCATTGAGGGCACCTGTGTTTGTAAACTA
CCGTATCAGTGCCCAAAGAATGGCACTGCAGTGTGTGCAACTAACAGGAGAAGC
TTCCCAACATACTGTCAACAAAAGAGTTTGGAATGTCTTCATCCAGGGACAAACT
TTTTAAATAACGGAACATGCACAGCCGAAGGAAAGTTTAGTGTTTCCTTGAAGCA
TGGAAATACAGATTCAGAGGGAATAGTTGAAGTAAAACTTGTGGACCAAGATAA
GACAATGTTCATATGCAAAAGCAGCTGGAGCATGAGGGAAGCCAACGTGGCCTG
CCTTGACCTTGGGTTTCAACAAGGTGCTGATACTCAAAGAAGGTTTAAGTTGTCT
GATCTCTCTATAAATTCCACTGAATGTCTACATGTGCATTGCCGAGGATTAGAGA
CCAGTTTGGCTGAATGTACTTTTACTAAGAGAAGAACTATGGGTTACCAGGATTT
CGCTGATGTGGTTTGTTATACACAGAAAGCAGATTCTCCAATGGATGACTTCTTT
CAGTGTGTGAATGGGAAATACATTTCTCAGATGAAAGCCTGTGATGGTATCAATG
ATTGTGGAGACCAAAGTGATGAACTGTGTTGTAAAGCATGCCAAGGCAAAGGCT
TCCATTGCAAATCGGGTGTTTGCATTCCAAGCCAGTATCAATGCAATGGTGAGGT
GGACTGCATTACAGGGGAAGATGAAGTTGGCTGTGCAGGCTTTGCATCTGTGACT
CAAGAAGAAACAGAAATTTTGACTGCTGACATGGATGCAGAAAGAAGACGGAT
AAAATCATTATTACCTAAACTATCTTGTGGAGTTAAAAACAGAATGCACATTCGA
AGGAAACGAATTGTGGGAGGAAAGCGAGCACAACTGGGAGACCTCCCATGGCA
GGTGGCAATTAAGGATGCCAGTGGAATCACCTGTGGGGGAATTTATATTGGTGG
CTGTTGGATTCTGACTGCTGCACATTGTCTCAGAGCCAGTAAAACTCATCGTTAC
CAAATATGGACAACAGTAGTAGACTGGATACACCCCGACCTTAAACGTATAGTA
ATTGAATACGTGGATAGAATTATTTTCCATGAAAACTACAATGCAGGCACTTACC
AAAATGACATCGCTTTGATTGAAATGAAAAAAGACGGAAACAAAAAAGATTGTG
AGCTGCCTCGTTCCATCCCTGCCTGTGTCCCCTGGTCTCCTTACCTATTCCAACCT
AATGATACATGCATCGTTTCTGGCTGGGGACGAGAAAAAGATAACGAAAGAGTC
TTTTCACTTCAGTGGGGTGAAGTTAAACTAATAAGCAACTGCTCTAAGTTTTACG
GAAATCGTTTCTATGAAAAAGAAATGGAATGTGCAGGTACATATGATGGTTCCAT
CGATGCCTGTAAAGGGGACTCTGGAGGCCCCTTAGTCTGTATGGATGCCAACAAT
GTGACTTATGTCTGGGGTGTTGTGAGTTGGGGGGAAAACTGTGGAAAACCAGAG
TTCCCAGGTGTTTACACCAAAGTGGCCAATTATTTTGACTGGATTAGCTACCATG
TAGGAAGGCCTTTTATTTCTCAGTACAATGTATAATAAGATATCGATACATTGAT
GAGTTTGGACAAACCACAACTAGAATGCAGTGAAAAAAATGCTTTATTTGTGAA
ATTTGTGATGCTATTGCTTTATTTGTAACCATTATAAGCTGCAATAAACAAGATAT
CGTTAACTCGAGGGATCCCACGTGCTGATTTTGTAGGTAACCACGTGCGGACCGA
GCGGCCGCAGGAACCCCTAGTGATGGAGTTGGCCACTCCCTCTCTGCGCGCTCGC
TCGCTCACTGAGGCCGGGCGACCAAAGGTCGCCCGACGCCCGGGCTTTGCCCGG
GCGGCCTCAGTGAGCGAGCGAGCGCGCAGCTGCCTGCAGGGGCGCCTGATGCGG
TATTTTCTCCTACGCATCTGTGCGGTATTTCACACCGCATACGTCAAAGCAACC
ATAGTACGCGCCCTGTAGCGGCGCATTAAGCGCGGCGGGTGTGGTGGTTACGCG
CAGCGTGACCGCTACACTTGCCAGCGCCCTAGCGCCCGCTCCTTTCGCTTTCTTCC
CTTCCTTTCTCGCCACGTTCGCCGGCTTTCCCCGTCAAGCTCTAAATCGGGGGCTC
CCTTTAGGGTTCCGATTTAGTGCTTTACGGCACCTCGACCCCAAAAAACTTGATT
TGGGTGATGGTTCACGTAGTGGGCCATCGCCCTGATAGACGGTTTTTCGCCCTTT
GACGTTGGAGTCCACGTTCTTTAATAGTGGACTCTTGTTCCAAACTGGAACAACA
CTCAACCCTATCTCGGGCTATTCTTTTGATTTATAAGGGATTTTGCCGATTTCGGC
CTATTGGTTAAAAAATGAGCTGATTTAACAAAAATTTAACGCGAATTTTAACAAA
ATATTAACGTTTACAATTTTATGGTGCACTCTCAGTACAATCTGCTCTGATGCCGC
ATAGTTAAGCCAGCCCCGACACCCGCCAACACCCGCTGACGCGCCCTGACGGGC
TTGTCTGCTCCCGGCATCCGCTACAGACAAGCTGTGACCGTCTCCGGGAGCTGC
ATGTGTCAGAGGTTTTCACCGTCATCACCGAAACGCGCGAGACGAAAGGGCCTC
GTGATACGCCTATTTTTATAGGTTAATGTCATGATAATAATGGTTTCTTAGACGTC
AGGTGGCACTTTTCGGGGAAATGTGCGCGGAACCCCTATTTGTTTATTTTTCTAA
ATACATTCAAATATGTATCCGCTCATGAGACAATAACCCTGATAAATGCTTCAAT
AATATTGAAAAAGGAAGAGTATGAGTATTCAACATTTCCGTGTCGCCCTTATTCC
CTTTTTTGCGGCATTTTGCCTTCCTGTTTTTGCTCACCCAGAAACGCTGGTGAAAG
TAAAAGATGCTGAAGATCAGTTGGGTGCACGAGTGGGTTACATCGAACTGGATC
TCAACAGCGGTAAGATCCTTGAGAGTTTTCGCCCCGAAGAACGTTTTCCAATGAT
GAGCACTTTTAAAGTTCTGCTATGTGGCGCGGTATTATCCCGTATTGACGCCGGG
CAAGAGCAACTCGGTCGCCGCATACACTATTCTCAGAATGACTTGGTTGAGTACT
CACCAGTCACAGAAAAGCATCTTACGGATGGCATGACAGTAAGAGAATTATGCA
GTGCTGCCATAACCATGAGTGATAACACTGCGGCCAACTTACTTCTGACAACGAT
CGGAGGACCGAAGGAGCTAACCGCTTTTTTGCACAACATGGGGGATCATGTAAC
TCGCCTTGATCGTTGGGAACCGGAGCTGAATGAAGCCATACCAAACGACGAGCG
TGACACCACGATGCCTGTAGCAATGGCAACAACGTTGCGCAAACTATTAACTGG
CGAACTACTTACTCTAGCTTCCCGGCAACAATTAATAGACTGGATGGAGGCGGAT
AAAGTTGCAGGACCACTTCTGCGCTCGGCCCTTCCGGCTGGCTGGTTTATTGCTG
ATAAATCTGGAGCCGGTGAGCGTGGGTCTCGCGGTATCATTGCACCACTGGGGC
CAGATGGTAAGCCCTCCCGTATCGTAGTTATCTACACGACGGGGAGTCAGGCAA
CTATGGATGAACGAAATAGACAGATCGCTGAGATAGGTGCCTCACTGATTAAGC
ATTGGTAACTGTCAGACCAAGTTTACTCATATATACTTTAGATTGATTTAAAACTT
CATTTTTAATTTAAAAGGATCTAGGTGAAGATCCTTTTTGATAATCTCATGACCA
AAATCCCTTAACGTGAGTTTTCGTTCCACTGAGCGTCAGACCCCGTAGAAAAGAT
CAAAGGATCTTCTTGAGATCCTTTTTTTCTGCGCGTAATCTGCTGCTTGCAAACAA
AAAAACCACCGCTACCAGCGGTGGTTTGTTTGCCGGATCAAGAGCTACCAACTCT
TTTTCCGAAGGTAACTGGCTTCAGCAGAGCGCAGATACCAAATACTGTCCTTCTA
GTGTAGCCGTAGTTAGGCCACCACTTCAAGAACTCTGTAGCACCGCCTACATACC
TCGCTCTGCTAATCCTGTTACCAGTGGCTGCTGCCAGTGGCGATAAGTCGTGTCT
TACCGGGTTGGACTCAAGACGATAGTTACCGGATAAGGCGCAGCGGTCGGGCTG
AACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAA
GGCGGACAGGTATCCGGTAAGCGGCAGGGTCGGAACAGGAGAGCGCACGAGGG
AGCTTCCAGGGGGAAACGCCTGGTATCTTTATAGTCCTGTCGGGTTTCGCCACCT
CTGACTTGAGCGTCGATTTTTGTGATGCTCGTCAGGGGGGCGGAGCCTATGGAAA
AACGCCAGCAACGCGGCCTTTTTACGGTTCCTGGCCTTTTGCTGGCCTTTTGCTCA
CATGT
SEQ ID NO: 29-CFI Amino Acid Sequence
MKLLHVFLLFLCFHLRFCKVTYTSQEDLVEKKCLAKKYTHLSCDKVFCQPWQRCIE
GTCVCKLPYQCPKNGTAVCATNRRSFPTYCQQKSLECLHPGTKFLNNGTCTAEGKFS
VSLKHGNTDSEGIVEVKLVDQDKTMFICKSSWSMREANVACLDLGFQQGADTQRRF
KLSDLSINSTECLHVHCRGLETSLAECTFTKRRTMGYQDFADVVCYTQKADSPMDD
FFQCVNGKYISQMKACDGINDCGDQSDELCCKACQGKGFHCKSGVCIPSQYQCNGE
VDCITGEDEVGCAGFASVTQEETEILTADMDAERRRIKSLLPKLSCGVKNRMHIRRK
RIVGGKRAQLGDLPWQVAIKDASGITCGGIYIGGCWILTAAHCLRASKTHRYQIWTT
VVDWIHPDLKRIVIEYVDRIIFHENYNAGTYQNDIALIEMKKDGNKKDCELPRSIPAC
VPWSFYLFQPNDTCIVSGWGREKDNERVFSLQWGEVKLISNCSKFYGNRFYEKEME
CAGTYDGSIDACKGDSGGPLVCMDANNVTYVWGVVSWGENCGKPEFPGVYTKVA
NYFDWISYHVGRPFISQYNV
SEQ ID NO: 30-CFH Amino Acid Sequence
MRLLAKIICLMLWAICVAEDCNELPPRRNTEILTGSWSDQTYPEGTQAIYKCRPGYRS
LG
NVIMVCRKGEWVALNPLRKCQKRPCGHPGDTPFGTFTLTGGNVFEYGVKAVYTCN
EGYQLLGEINYRECDTDGWTNDIPICEVVKCLPVTAPENGKIVSSAMEPDREYHFGQ
AVRFVCNSGYKIEGDEEMHCSDDGFWSKEKPKCVEISCKSPDVINGSPISQKIIYKEN
ERFQYKCNMGYEYSERGDAVCTESGWRPLPSCEEKSCDNPYIPNGDYSPLRIKHRTG
DEITYQCRNGFYPATRGNTAKCTSTGWIPAPRCTLKPCDYPDIKHGGLYHENMRRPY
FPVAVGKYYSYYCDEHFETPSGSYWDHIHCTQDGWSPAVPCLRKCYFPYLENGYNQ
NYGRKFVQGKSIDVACHPGYALPKAQTTVTCMENGWSPTPRCIRVKTCSKSSIDIEN
GFISESQYTYALKEKAKYQCKLGYVTADGETSGSITCGKDGWSAQPTCIKSCDIPVF
MNARTKNDFTWFKLNDTLDYECHDGYESNTGSTTGSIVCGYNCWSDLPICYERECE
LPKIDVHLVPDRKKDQYKVGEVLKFSCKPGFTIVGPNSVQCYHFGLSPDLPICKEQV
QSCGPPPELLNGNVKEKTKEEYGHSEVVEYYCNPRFLMKGPNKIQCVDGEWTTLPV
CIVEESTCGDIPELEHGWAQLSSPPYYYGDSVEFNCSESFTMIGHRSITCIHGVWTQLP
QCVAIDKLKKCKSSNLIILEEHLKNKKEFDHNSNIRYRCRGKEGWIHTVCINGRWDP
EVNCSMAQIQLCPPPPQIPNSHNMTTTLNYRDGEKVSVLCQENYLIQEGEEITCKDGR
WQSIPLCVEKIPCSQPPQIEHGTINSSRSSQESYAHGTKLSYTCEGGFRISEENETTCYM
GKWSSPPQCEGLPCKSPPEISHGVVAHMSDSYQYGEEVTYKCFEGFGIDGPAIAKCL
GEKWSHPPSCIKTDCLSLPSFENAIPMGEKKDVYKAGEQVTYTCATYYKMDGASNV
TCINSRWTGRPTCRDTSCVNPPTVQNAYIVSRQMSKYPSGERVRYQCRSPYEMFGDE
EVMCLNGNWTEPPQCKDSTGKCGPPPPIDNGDITSFPLSVYAPASSVEYQCQNLYQL
EGNKRITCRNGQWSEPPKCLHPCVISREIMENYNIALRWTAKQKLYSRTGESVEFVC
KRGYRLSSRSHTLRTTCWDGKLEYPTCAK
SEQ ID NO: 31-FHL1 Amino Acid Sequence
MRLLAKIICLMLWAICVAEDCNELPPRRNTEILTGSWSDQTYPEGTQAIYKCRPGYRS
LG
NVIMVCRKGEWVALNPLRKCQKRPCGHPGDTPFGTFTLTGGNVFEYGVKAVYTCN
EGYQLLGEINYRECDTDGWTNDIPICEVVKCLPVTAPENGKIVSSAMEPDREYHFGQ
AVRFVCNSGYKIEGDEEMHCSDDGFWSKEKPKCVEISCKSPDVINGSPISQKIIYKEN
ERFQYKCNMGYEYSERGDAVCTESGWRPLPSCEEKSCDNPYIPNGDYSPLRIKHRTG
DEITYQCRNGFYPATRGNTAKCTSTGWIPAPRCTLKPCDYPDIKHGGLYHENMRRPY
FPVAVGKYYSYYCDEHFETPSGSYWDHIHCTQDGWSPAVPCLRKCYFPYLENGYNQ
NYGRKFVQGKSIDVACHPGYALPKAQTTVTCMENGWSPTPRCIRVSFTL
SEQ ID NO: 32-MECP Promoter Sequence
GGCCGAAATGGACAGGAAATCTCGCCAATTGACGGCATCGCCGCTGAGACTCCC
CCCTCCCCCGTCCTCCCCGTCCCAGCCCGGCCATCACAGCCAATGACGGGCGGGC
TCGCAGCGGCGCCGAGGGCGGGGCGCGGGCGCGCAGGTGCAGCAGCGCGCGGG
CCGGCCAAGAGGGCGGGGCGCGACGTCGGCCGTGCGGGGTCCCGGCGTCGGCGG
CGCGCGC
SEQ ID NO: 33-Representative CFI AAV Vector (with CBA Promoter)
CCTGCAGGCAGCTGCGCGCTCGCTCGCTCACTGAGGCCGCCCGGGCGTCGGGCG
ACCTTTGGTCGCCCGGCCTCAGTGAGCGAGCGAGCGCGCAGAGAGGGAGTGGCC
AACTCCATCACTAGGGGTTCCTGCGGCCGCACGCGTGTTAACTAGTGGCCCGCCT
GGCTGACCGCCCAACGACCCCCGCCCATTGACGTCAATAATGACGTATGTTCCCA
TAGTAACGCCAATAGGGACTTTCCATTGACGTCAATGGGTGGACTATTTACGGTA
AACTGCCCACTTGGCAGTACATCAAGTGTATCATATGCCAAGTACGCCCCCTATT
GACGTCAATGACGGTAAATGGCCCGCCTGGCATTATGCCCAGTACATGACCTTAT
GGGACTTTCCTACTTGGCAGTACATCTACGTATTAGTCATCGCTATTACCATGGTC
GAGGTGAGCCCCACGTTCTGCTTCACTCTCCCCATCTCCCCCCCCTCCCCACCCCC
AATTTTGTATTTATTTATTTTTTAATTATTTTGTGCAGCGATGGGGGCGGGGGGGG
GGGGGGGGCGCGCGCCAGGCGGGGCGGGGCGGGGCGAGGGGCGGGGCGGGGC
GAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCG
GGCGGGAGTCGCTGCGACGCTGCCTTCGCCCCGTGCCCCGCTCCGCCGCCGCCTC
GCGCCGCCCGCCCCGGCTCTGACTGACCGCGTTACTCCCACAGGTGAGCGGGCG
GGACGGCCCTTCTCCTCCGGGCTGTAATTAGCGCTTGGTTTAATGACGGCTTGTTT
CTTTTCTGTGGCTGCGTGAAAGCCTTGAGGGGCTCCGGGAGGGCCCTTTGTGCGG
GGGGGAGCGGCTCGGGGGGTGCGTGCGTGTGTGTGTGCGTGGGGAGCGCCGCGT
GCGGCCCGCGCTGCCCGGCGGCTGTGAGCGCTGCGGGCGCGGCGCGGGGCTTTG
TGCGCTCCGCAGTGTGCGCGAGGGGAGCGCGGCCGGGGGCGGTGCCCCGCGGTG
CGGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCC
CCTCCCCGAGTTGCTGAGCACGGCCCGGCTTCGGGTGCGGGGCTCCGTACGGGG
CGTGGCGCGGGGCTCGCCGTGCCGGGCGGGGGGTGGCGGCAGGTGGGGGTGCCG
GGCGGGGCGGGGCCGCCTCGGGCCGGGGAGGGCTCGGGGGAGGGGCGCGGCGG
CCCCCGGAGCGCCGGCGGCTGTCGAGGCGCGGCGAGCCGCAGCCATTGCCTTTT
ATGGTAATCGTGCGAGAGGGCGCAGGGACTTCCTTTGTCCCAAATCTGTGCGGA
GCCGAAATCTGGGAGGCGCCGCCGCACCCCCTCTAGCGGGCGCGGGGCGAAGCG
GTGCGGCGCCGGCAGGAAGGAAATGGGCGGGGAGGGCCTTCGTGCGTCGCCGCG
CCGCCGTCCCCTTCTCCCTCTCCAGCCTCGGGGCTGTCCGCGGGGGGACGGCTGC
CTTCGGGGGGGACGGGGCAGGGCGGGGTTCGGCTTCTGGCGTGTGACCGGCGGC
TCTAGAGCCTCTGCTAACCATGTTCATGCCTTCTTCTTTTTCCTACAGCTCCTGGG
CAACGTGCTGGTTATTGTGCTGTCTCATCATTTTGGCAAAACCGGTCTCGAAGGC
CTGCAGGCGGCCGCCGCCACCATGAAGCTTCTTCATGTTTTCCTGTTATTTCTGTG
CTTCCACTTAAGGTTTTGCAAGGTCACTTATACATCTCAAGAGGATCTGGTGGAG
AAAAAGTGCTTAGCAAAAAAATATACTCACCTCTCCTGCGATAAAGTCTTCTGCC
AGCCATGGCAGAGATGCATTGAGGGCACCTGTGTTTGTAAACTACCGTATCAGTG
CCCAAAGAATGGCACTGCAGTGTGTGCAACTAACAGGAGAAGCTTCCCAACATA
CTGTCAACAAAAGAGTTTGGAATGTCTTCATCCAGGGACAAAGTTTTTAAATAAC
GGAACATGCACAGCCGAAGGAAAGTTTAGTGTTTCCTTGAAGCATGGAAATACA
GATTCAGAGGGAATAGTTGAAGTAAAACTTGTGGACCAAGATAAGACAATGTTC
ATATGCAAAAGCAGCTGGAGCATGAGGGAAGCCAACGTGGCCTGCCTTGACCTT
GGGTTTCAACAAGGTGCTGATACTCAAAGAAGGTTTAAGTTGTCTGATCTCTCTA
TAAATTCCACTGAATGTCTACATGTGCATTGCCGAGGATTAGAGACCAGTTTGGC
TGAATGTACTTTTACTAAGAGAAGAACTATGGGTTACCAGGATTTCGCTGATGTG
GTTTGTTATACACAGAAAGCAGATTCTCCAATGGATGACTTCTTTCAGTGTGTGA
ATGGGAAATACATTTCTCAGATGAAAGCCTGTGATGGTATCAATGATTGTGGAGA
CCAAAGTGATGAACTGTGTTGTAAAGCATGCCAAGGCAAAGGCTTCCATTGCAA
ATCGGGTGTTTGCATTCCAAGCCAGTATCAATGCAATGGTGAGGTGGACTGCATT
ACAGGGGAAGATGAAGTTGGCTGTGCAGGCTTTGCATCTGTGGCTCAAGAAGAA
ACAGAAATTTTGACTGCTGACATGGATGCAGAAAGAAGACGGATAAAATCATTA
ITACCTAAACTATCTTGTGGAGTTAAAAACAGAATGCACATTCGAAGGAAACGA
ATTGTGGGAGGAAAGCGAGCACAACTGGGAGACCTCCCATGGCAGGTGGCAATT
AAGGATGCCAGTGGAATCACCTGTGGGGGAATTTATATTGGTGGCTGTTGGATTC
TGACTGCTGCACATTGTCTCAGAGCCAGTAAAACTCATCGTTACCAAATATGGAC
AACAGTAGTAGACTGGATACACCCCGACCTTAAACGTATAGTAATTGAATACGT
GGATAGAATTATTTTCCATGAAAACTACAATGCAGGCACTTACCAAAATGACATC
GCTTTGATTGAAATGAAAAAAGACGGAAACAAAAAAGATTGTGAGCTGCCTCGT
TCCATCCCTGCCTGTGTCCCCTGGTCTCCTTACCTATTCCAACCTAATGATACATG
CATCGTTTCTGGCTGGGGACGAGAAAAAGATAACGAAAGAGTCTTTTCACTTCA
GTGGGGTGAAGTTAAACTAATAAGCAACTGCTCTAAGTTTTACGGAAATCGTTTC
TATGAAAAAGAAATGGAATGTGCAGGTACATATGATGGTTCCATCGATGCCTGT
AAAGGGGACTCTGGAGGCCCCTTAGTCTGTATGGATGCCAACAATGTGACTTATG
TCTGGGGTGTTGTGAGTTGGGGGGAAAACTGTGGAAAACCAGAGTTCCCAGGTG
TTTACACCAAAGTGGCCAATTATTTTGACTGGATTAGCTACCATGTAGGAAGGCC
TTTTATTTCTCAGTACAATGTATAATAAGATATCGATACATTGATGAGTTTGGAC
AAACCACAACTAGAATGCAGTGAAAAAAATGCTTTATTTGTGAAATTTGTGATGC
TATTGCTTTATTTGTAACCATTATAAGCTGCAATAAACAAGATATCGTTAACTCG
AGGGATCCCACGTGCGGACCGAGCGGCCGCAGGAACCCCTAGTGATGGAGTTGG
CCACTCCCTCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGCGCAGCTG
CCTGCAGGGGCGCCTGATGCGGTATTTTCTCCTTACGCATCTGTGCGGTATTTCAC
ACCGCATAGGTCAAAGCAACCATAGTACGCGCCCTGTAGCGGCGCATTAAGGGC
GGCGGGTGTGGTGGTTACGCGCAGCGTGACCGCTACACTTGCCAGCGCCTTAGC
GCCCGCTCCTTTCGCTTTCTTCCCTTCCTTTCTCGCCACGTTCGCCGGCTTTCCCCG
TCAAGCTCTAAATCGGGGGCTCCCTTTAGGGTTCCGATTTTAGTGCTTTACGGCAC
CTCGACCCCAAAAAACTTGATTTGGGTGATGGTTCACGTAGTGGGCCATCGCCCT
GATAGACGGTTTTTCGCCCTTTGACGTTGGAGTCCACGTTCTTTAATAGTGGACTC
TTGTTCCAAACTGGAACAACACTCAACTCTATCTCGGGCTATTCTTTTGATTTATA
AGGGATTTTGCCGATTTCGGTCTATTGGTTAAAAAATGAGCTGATTTAACAAAAA
TTTAACGCGAATTTTAACAAAATATTAACGTTTACAATTTTATGGTGCACTCTCAG
TACAATCTGCTCTGATGCCGCATAGTTAAGCCAGCCCCGACACCCGCCAACACCC
GCTGACGCGCCCTGACGGGCTTGTCTGCTCCCGGCATCCGCTTACAGACAAGCTG
TGACCGTCTCCGGGAGCTGCATGTGTCAGAGGTTTTCACCGTCATCACCGAAACG
CGCGAGACGAAAGGGCCTCGTGATACGCCTATTTTTATAGGTTAATGTCATGATA
ATAATGGTTTCTTAGACGTCAGGTGGCACTTTTCGGGGAAATGTGCGCGGAACCC
CTATTTGTTTATTTTTCTAAATACATTCAAATATGTATCCGCTCATGAGACAATAA
CCCTGATAAATGCTTCAATAATATTGAAAAAGGAAGAGTatgagccatattcaacgggaaacgt
cgaggccgcgattaaattccaacatggatgctgatttatatgggtataaatgggctcgcgataatgtcgggcaatcaggtgcgacaatct
atcgcttgtatgggaagcccgatgcgccagagttgtttctgaaacatggcaaaggtagcgttgccaatgatgttacagatgagatggtc
agactaaactggctgacggaatttatgcctcttccgaccatcaagcattttatccgtactcctgatgatgcatggttactcaccactgcgat
ccccggaaaaacagcattccaggtattagaagaatatcctgattcaggtgaaaatattgttgatgcgctggcagtgttcctgcgccggtt
gcattcgattcctgtttgtaattgtccttttaacagcgatcgcgtatttcgtctcgctcaggcgcaatcacgaatgaataacggtttggttg
atgcgagtgattttgatgacgagcgtaatggctggcctgttgaacaagtctggaaagaaatgcataaacttttgccattctcaccggattcag
tcgtcactcatggtgatttctcacttgataaccttatttttgacgaggggaaattaataggttgtattgatgttggacgagtcggaatcgcag
accgataccaggatcttgccatcctatggaactgcctcggtgagttttctccttcattacagaaacggctttttcaaaaatatggtattgata
atcctgatatgaataaattgcagtttcatttgatgctcgatgagtttttctaaCTGTCAGACCAAGTTTACTCATATA
TACTTTAGATTGATTTAAAACTTCATTTTTAATTTAAAAGGATCTAGGTGAAGATC
CTTTTTGATAATCTCATGACCAAAATCCCTTAACGTGAGTTTTCGTTCCACTGAGC
GTCAGACCCCGTAGAAAAGATCAAAGGATCTTCTTGAGATCCTTTTTTTCTGCGC
GTAATCTGCTGCTTGCAAACAAAAAAACCACCGCTACCAGCGGTGGTTTGTTTGC
CGGATCAAGAGCTACCAACTCTTTTTCCGAAGGTAACTGGCTTCAGCAGAGCGCA
GATACCAAATACTGTTCTTCTAGTGTAGCCGTAGTTAGGCCACCACTTCAAGAAC
TCTGTAGCACCGCCTACATACCTCGCTCTGCTAATCCTGTTACCAGTGGCTGCTGC
CAGTGGCGATAAGTCGTGTCTTACCGGGTTGGACTCAAGACGATAGTTACCGGAT
AAGGCGCAGCGGTCGGGCTGAACGGGGGGTTCGTGCACACAGCCCAGCTTGGAG
CGAACGACCTACACCGAACTGAGATACCTACAGCGTGAGCTATGAGAAAGCGCC
ACGCTTCCCGAAGGGAGAAAGGCGGACAGGTATCCGGTAAGCGGCAGGGTCGG
AACAGGAGAGCGCACGAGGGAGCTTCCAGGGGGAAACGCCTGGTATCTTTATAG
TCCTGTCGGGTTTCGCCACCTCTGACTTGAGCGTCGATTTTTGTGATGCTCGTCAG
GGGGGCGGAGCCTATGGAAAAACGCCAGCAACGCGGCCTTTTTACGGTTCCTGG
CCTTTTCCTGGCCTTTTGCTCACATGT
SEQ ID NO: 34-Exemplary CFI Nucleotide Sequence
ATGAAGCTTCTTCATGTTTTCCTGTTATTTCTGTGCTTCCACTTAAGGTTTTGCAA
GGTCACTTATACATCTCAAGAGGATCTGGTGGAGAAAAAGTGCTTAGCAAAAAA
ATATACTCACCTCTCCTGCGATAAAGTCTTCTGCCAGCCATGGCAGAGATGCATT
GAGGGCACCTGTGTTTGTAAACTACCGTATCAGTGCCCAAAGAATGGCACTGCA
GTGTGTGCAACTAACAGGAGAAGCTTCCCAACATACTCTCAACAAAAGAGTTTG
GAATGTCTTCATCCAGGGACAAAGTTTTTAAATAACGGAACATGCACAGCCGAA
GGAAAGTTTAGTGTTTCCTTGAAGCATGGAAATACAGATTCAGAGGGAATAGTT
GAAGTAAAACTTGTGGACCAAGATAAGACAATGTTCATATGCAAAAGCAGCTGG
AGCATGAGGGAAGCCAACGTGGCCTGCCTTGACCTTGGGTTTCAACAAGGTGCT
GATACTCAAAGAAGGTTTAAGTTGTCTGATCTCTCTATAAATTCCACTGAATGTC
TACATGTGCATTGCCGAGGATTAGAGACCAGTTTGGCTGAATGTACTTTTACTAA
GAGAAGAACTATGGGTTACCAGGATTTCGCTGATGTGGTTTGTTATACACAGAAA
GCAGATTCTCCAATGGATGACTTCTTTCAGTGTGTGAATGGGAAATACATTTCTC
AGATGAAAGCCTGTGATGGTATCAATGATTGTGGAGACCAAAGTGATGAACTGT
GTTGTAAAGCATGCCAAGGCAAAGGCTTCCATTGCAAATCGGGTGTTTGCATTCC
AAGCCAGTATCAATGCAATGGTGAGGTGGACTGCATTACAGGGGAAGATGAAGT
TGGCTGTGCAGGCTTTGCATCTGTGGGCAAGAAGAAACAGAAATTTTGACTGCT
GACATGGATGCAGAAAGAAGACGGATAAAATCATTATTACCTAAACTATCTTGT
GGAGTTAAAAACAGAATGCACATTCGAAGGAAACGAATTGTGGGAGGAAAGCG
AGCACAACTGGGAGACCTCCCATGGCAGGTGGCAATTAAGGATGCCAGTGGAAT
CACCTGTGGGGGAATTTATATTGGTGGCTGTTGGATTCTGACTGCTGCACATTGT
CTCAGAGCCAGTAAAACTCATCGTTACCAAATATGGACAACAGTAGTAGACTGG
ATACACCCCGACCTTAAACGTATAGTAATTGAATACGTGGATAGAATTATTTTCC
ATGAAAACTACAATGCAGGCACTTACCAAAATGACATCGCTTTGATTGAAATGA
AAAAAGACGGAAACAAAAAAGATTGTGAGCTGCCTCGTTCCATCCCTGCCTGTG
TCCCCTGGTCTCCTTACCTATTCCAACCTAATGATACATGCATCGTTTCTGGCTGG
GGACGAGAAAAAGATAACGAAAGAGTCTTTTCACTTCAGTGGGGTGAAGTTAAA
CTAATAAGCAACTGCTCTAAGTTTTACGGAAATCGTTTCTATGAAAAAGAAATGG
AATGTGCAGGTACATATGATGGTTCCATCGATGCCTGTAAAGGGGACTCTGGAG
GCCCCTTAGTCTGTATGGATGCCAACAATGTGACTTATGTCTGGGGTGTTGTGAG
TTGGGGGGAAAACTGTGGAAAACCAGAGTTCCCAGGTGTTTACACCAAAGTGGC
CAATTATTTTGACTGGATTAGCTACCATGTAGGAAGGCCTTTTATTTCTCAGTACA
ATGTATAA
SEQ ID NO: 35-Exemplary CFI amino acid sequence
MKLLHVFLLFLCFHLRFCKVTYTSQEDLVEKKCLAKKYTHLSCDKVFCQPWQRCIE
GTCVCKLPYQCPKNGTAVCATNRRSFPTYCQQKSLECLHPGTKFLNNGTCTAEGKFS
VSLKHGNTDSEGIVEVKLVDQDKTMFICKSSWSMREANVACLDLGFQQGADTQRRF
KLSDLSINSTECLMVMCRGLETSLAECTFTKRRTMGYQDFADVVCYTQKADSPMDD
FFQCVNGKYISQMKACDGINDCGDQSDELCCKACQGKGFHCKSGVCIPSQYQCNGE
VDCITGEDEVGCAGFASVAQEETEILTADMDAERRRIKSLLPKLSCGVKNRMHIRRK
RIVGGKRAQLGDLPWQVAIKDASGITCGGIYIGGCWILTAAHCLRASKTHRYQIWTT
VVDWIHPDLKRIVIEYVDRIIFHENYNAGTYQNDIALIEMKKDGNKKDCELPRSIPAC
VPWSPYLFQPNDTCIVSGWGREKDNERVFSLQWGEVKLISNCSKFYGNRFYEKEME
CAGTYDGSIDACKGDSGGPLVCMDANNVTYVWGVVSVVGENCGKPEFPGVYTKVA
NYFDWISYHVGRPFISQYNV

1. An adeno-associated viral (AAV) vector encoding a human Complement Factor I (CFI) protein or biologically active fragment thereof, wherein the vector comprises a nucleotide sequence that is at least 70% identical to the nucleotide sequence of SEQ ID NO: 1-3, 5 or 34, or codon-optimized variant and/or a fragment thereof.

2. The AAV vector of claim 1, wherein the nucleotide sequence is at least 90% identical to the nucleotide sequence of SEQ ID NO: 1-3, 5 or 34, or codon-optimized variant and/or a fragment thereof.

3. The AAV vector of claim 1, wherein the nucleotide sequence is at least 95% identical to the nucleotide sequence of SEQ ID NO: 1-3, 5 or 34, or codon-optimized variant and/or a fragment thereof.

4. The AAV vector of claim 1, wherein the nucleotide sequence is the sequence of SEQ ID NO: 1-3, 5 or 34, or codon-optimized variant and/or a fragment thereof.

5. The AAV vector of claim 1, wherein the nucleotide sequence is at least 701%, 75%, 80%, 85%, 90%, 91%, 92%, 93%, 94%, 95%, 96%, 97%, 98%, 99% or 100% identical to the sequence of SEQ ID NO: 34.

6. The AAV vector of any one of claims 1-5, wherein the vector encodes a CFI protein or biologically active fragment thereof comprising a heavy chain and a light chain.

7. The AAV vector of any one of claims 1-6, wherein the vector encodes a CFI protein or biologically active fragment thereof comprising a FIMAC domain.

8. The AAV vector of any one of claims 1-7, wherein the vector encodes a CFI protein or biologically active fragment thereof comprising a Scavenger Receptor Cysteine Rich (SRCR) domain.

9. The AAV vector of any one of claims 1-8, wherein the vector encodes a CFI protein or biologically active fragment thereof comprising at least one LDL receptor Class A domain.

10. The AAV vector of any one of claims 1-9, wherein the vector encodes a CFI protein or biologically active fragment thereof comprising two LDL receptor Class A domains.

11. The AAV vector of any one of claims 1-10, wherein the vector encodes a CFI protein or biologically active fragment thereof comprising a serine protease domain.

12. The AAV vector of any one of claims 1-11, wherein the vector encodes a CFI protein or biologically active fragment thereof comprising a FIMAC domain, a Scavenger Receptor Cysteine Rich (SRCR) domain, and two LDL receptor Class A domains.

13. The AAV vector of any one of claims 1-12, wherein the vector encodes a CFI protein or biologically active fragment thereof capable of cleaving C3b and C4b proteins.

14. The AAV vector of any one of claims 1-13, wherein the vector encodes a CFI protein or biologically active fragment thereof capable of inhibiting the assembly of C3 and C5 convertase enzymes.

15. The AAV vector of any one of claims 1-14, wherein the vector comprises a promoter that is at least 1000 nucleotides in length.

16. The AAV vector of any one of claims 1-15, wherein the vector comprises a promoter that is at least 1500 nucleotides in length.

17. The AAV vector of any one of claims 1-16, wherein the promoter comprises a nucleotide sequence that is at least 70%, 75%, 80%, 85%, 90%, 91%, 92%, 93%, 94%, 95%, 96%, 97%, 98%, 99%, or 100% identical to the nucleotide sequence of any one of SEQ ID NOs: 8, 9, 11, 12, 13, 15, 17, 21, 23, 25, or 27.

18. The AAV vector of any one of claims 1-17, wherein the promoter comprises a promoter having the nucleotide sequence of SEQ ID NO: 8, or a fragment thereof.

19. The AAV vector of any one of claims 1-17, wherein the promoter comprises a promoter having the nucleotide sequence of SEQ ID NO: 9, or a fragment thereof.

20. The AAV vector of any one of claims 1-17, wherein the promoter comprises a promoter having the nucleotide sequence of SEQ ID NO: 11, or a fragment thereof.

21. The AAV vector of any one of claims 1-17, wherein the promoter comprises a promoter having the nucleotide sequence of SEQ ID NO: 12, or a fragment thereof.

22. The AAV vector of any one of claims 1-17, wherein the promoter comprises a promoter having the nucleotide sequence of SEQ ID NO: 13, or a fragment thereof.

23. The AAV vector of any one of claims 1-17, wherein the promoter comprises a promoter having the nucleotide sequence of SEQ ID NO: 15, or a fragment thereof.

24. The AAV vector of any one of claims 1-17, wherein the promoter comprises a promoter having the nucleotide sequence of SEQ ID NO: 17, or a fragment thereof.

25. The AAV vector of any one of claims 1-17, wherein the promoter comprises a promoter having the nucleotide sequence of SEQ ID NO: 21, or a fragment thereof.

26. The AAV vector of any one of claims 1-17, wherein the promoter comprises a promoter having the nucleotide sequence of SEQ ID NO: 23, or a fragment thereof.

27. The AAV vector of any one of claims 1-17, wherein the promoter comprises a promoter having the nucleotide sequence of SEQ ID NO: 25, or a fragment thereof.

28. The AAV vector of any one of claims 1-17, wherein the promoter comprises a promoter having the nucleotide sequence of SEQ ID NO: 27, or a fragment thereof.

29. The AAV vector of any one of claims 1-28, wherein the vector is an AAV2 vector.

30. The AAV vector of any one of claims 1-29, wherein the vector is an AAV8 vector.

31. The AAV vector of any one of claims 1-30, wherein the vector comprises a CMV promoter.

32. The AAV vector of any one of claims 1-31, wherein the vector comprises a Kozak sequence.

33. The AAV vector of any one of claims 1-32, wherein the vector comprises one or more ITR sequence flanking the vector portion encoding CFI.

34. The AAV vector of any one of claims 1-33, wherein the vector comprises a polyadenylation sequence.

35. The AAV vector of any one of claims 1-34, wherein the vector comprises a selective marker.

36. The AAV vector of claim 35, wherein the selective marker is an antibiotic-resistance gene.

37. The AAV vector of claim 36, wherein the antibiotic-resistance gene is an ampicillin-resistance gene.

38. The AAV vector of claim 36, wherein the antibiotic-resistance gene is a kanamycin-resistance gene.

39. A composition comprising the AAV vector of any one of claims 1-38 and a pharmaceutically acceptable carrier.

40. The composition of claim 39, wherein the composition does not comprise a protease or a polynucleotide encoding a protease.

41. The composition of claim 40, wherein the composition does not comprise a furin protease or a polynucleotide encoding a furin protease.

42. A method of treating a subject having a disorder associated with undesired activity of the alternative complement pathway, comprising the step of administering to the subject any of the vectors of any one of claims 1-38 or 111-119 or the compositions of any one of claims 39-41.

43. A method of treating a subject having age-related macular degeneration (AMD), comprising the step of administering to the subject any of the vectors of any one of claims 1-38 or 111-119 or the compositions of any one of claims 39-41.

44. The method of claim 42 or 43, wherein the vector or composition is administered intravitreally.

45. The method of any of claims 42-44, wherein the subject is not administered a protease or a polynucleotide encoding a protease.

46. The method of any of claims 42-44, wherein the subject is not administered a furin protease or a polynucleotide encoding a furin protease.

47. The method of any one of claims 42-46, wherein the subject is a human.

48. The method of claim 47, wherein the human is at least 40 years of age.

49. The method of claim 47, wherein the human is at least 50 years of age.

50. The method of claim 47, wherein the human is at least 65 years of age.

51. The method of any one of claims 42-50, wherein the vector or composition is administered locally.

52. The method of any one of claims 42-50, wherein the vector or composition is administered systemically.

53. The method of any one of claims 42-52, wherein the vector or composition comprises a promoter that is associated with strong expression in the liver.

54. The method of claim 53, wherein the promoter comprises a nucleotide sequence that is at least 90%, 95% or 100% identical to the nucleotide sequence of any one of SEQ ID NOs: 13, 15 or 27.

55. The method of any one of claims 42-54, wherein the vector or composition comprises a promoter that is associated with strong expression in the eye.

56. The method of claim 55, wherein the promoter comprises a nucleotide sequence that is at least 90%, 95%, or 100% identical to the nucleotide sequence of any one of SEQ ID NOs: 21 or 25.

57. The method of any one of claims 42-56, wherein the subject has a loss-of-function mutation in the subject's CFI gene.

58. The method of any one of claims 42-57, wherein the subject has one or more CFI mutations selected from the group consisting of: G119R, L131R, V152M, G162D, R187Y, R187T, T203I, A240G, A258T, G287R, A300T, R317W, R339Q, V412M, and P553S.

59. The method of any one of claims 42-57, wherein the subject has a P553S CF mutation.

60. The method of any one of claims 42-57, wherein the subject has a K441R CFI mutation.

61. The method of any one of claims 42-57, wherein the subject has an R339Q CF mutation.

62. The method of any one of claims 42-57, wherein the subject has an R339Ter CFI mutation.

63. The method of any one of claims 42-57, wherein the subject has an R317Q CF mutation.

64. The method of any one of claims 42-57, wherein the subject has an R317W CFI mutation.

65. The method of any one of claims 42-57, wherein the subject has an A300T CFI mutation.

66. The method of any one of claims 42-57, wherein the subject has a G287R CFI mutation.

67. The method of any one of claims 42-57, wherein the subject has a G261D CFI mutation.

68. The method of any one of claims 42-57, wherein the subject has an A258T CFI mutation.

69. The method of any one of claims 42-57, wherein the subject has an A240G CFI mutation.

70. The method of any one of claims 42-57, wherein the subject has a T203I CFI mutation.

71. The method of any one of claims 42-57, wherein the subject has an R187Q CFI mutation.

72. The method of any one of claims 42-57, wherein the subject has an R187Ter CFI mutation.

73. The method of any one of claims 42-57, wherein the subject has a G162D CFI mutation.

74. The method of any one of claims 42-57, wherein the subject has a V152M CFI mutation.

75. The method of any one of claims 42-57, wherein the subject has a G119R CFI mutation.

76. The method of any one of claims 57-75, wherein the subject is homozygous for the CFI mutation.

77. The method of any one of claims 57-75, wherein the subject is heterozygous for the CFI mutation.

78. The method of any one of claims 57-77, wherein the subject expresses a mutant CF protein having reduced CFI activity as compared to a wildtype CFI protein (e.g., a CFI protein having the amino acid sequence of SEQ ID NO: 29).

79. The method of claim 78, wherein the CFI activity is the ability to cleave C3b to iC3b.

80. The method of any one of claims 57-79, wherein if a CFI protein having the CFI mutation were tested in a functional assay, the mutant CFI protein would display reduced CFI activity as compared to a wildtype CFI protein (e.g., a CFI protein having the amino acid sequence of SEQ ID NO: 29).

81. The method of claim 80, wherein the functional assay tests the ability of CFI to cleave C3b to iC3b.

82. The method of any one of claims 42-81, wherein the subject has a loss-of-function mutation in the subject's CFH gene.

83. The method of any one of claims 42-82, wherein the subject has one or more CFH mutations selected from the group consisting of: R2T, L3V, R53C, R53H, S58A, G69E, D90G, R175Q, S193L, I216T, I221V, R303W, H402Y, Q408X, P503A, G650V, R1078S, and R1210C.

84. The method of any one of claims 42-83, wherein the subject has atypical hemolytic uremic syndrome (aHUS).

85. The method of any one of claims 42-84, wherein the subject is suffering from a renal disease or complication.

86. The method of any one of claims 42-85, wherein the vector or composition is administered to the retina at a dose in the range of 1×10¹⁰ vg/eye to 1×10¹¹ vg/eye.

87. The method of claim 86, wherein the vector or composition is administered to the retina at a dose of about 1.4×10¹¹ vg/eye.

88. The method of any one of claims 42-87, wherein the CFI is processed to an active CFI.

89. The method of any one of claims 1-88, wherein the subject is a subject in whom it has been determined has one or more CFI mutations.

90. The method of claim 89, wherein the subject is a subject in whom it has been determined has one or more CFI mutations selected from the group consisting of: G119R, L131R, V152M, G162D, R187Y, R187T, I203I, A240G, A258T, G287R, A300T, R317W, R339Q, V412M, and P553S.

91. The method of claim 89, wherein the subject is a subject in whom it has been determined has one or more CFI mutations selected from the group consisting of: P553S, K441R, R339Q, R339Ter, R317Q, R317W, A300T, G287R, G261D, A258T, A240G, T203I, R187Q, R187Ter, G162D, V152M, or G119R.

92. The method of claim 89, wherein the subject is a subject in whom it has been determined has a P553S CF mutation.

93. The method of claim 89, wherein the subject is a subject in whom it has been determined has a K441R CFI mutation.

94. The method of claim 89, wherein the subject is a subject in whom it has been determined has an R339Q CFI mutation.

95. The method of claim 89, wherein the subject is a subject in whom it has been determined has an R339Ter CFI mutation.

96. The method of claim 89, wherein the subject is a subject in whom it has been determined has an R317Q CFI mutation.

97. The method of claim 89, wherein the subject is a subject in whom it has been determined has an R317W CF mutation.

98. The method of claim 89, wherein the subject is a subject in whom it has been determined has an A300T CF mutation.

99. The method of claim 89, wherein the subject is a subject in whom it has been determined has a G287R CFI mutation.

100. The method of claim 89, wherein the subject is a subject in whom it has been determined has a G261D CFI mutation.

101. The method of claim 89, wherein the subject is a subject in whom it has been determined has an A258T CFI mutation.

102. The method of claim 89, wherein the subject is a subject in whom it has been determined has an A240G CFI mutation.

103. The method of claim 89, wherein the subject is a subject in whom it has been determined has a T203I CF mutation.

104. The method of claim 89, wherein the subject is a subject in whom it has been determined has an R187Q CFI mutation.

105. The method of claim 89, wherein the subject is a subject in whom it has been determined has an R187Ter CFI mutation.

106. The method of claim 89, wherein the subject is a subject in whom it has been determined has a G162D CFI mutation.

107. The method of claim 89, wherein the subject is a subject in whom it has been determined has a V152M CFI mutation.

108. The method of claim 89, wherein the subject is a subject in whom it has been determined has a G119R CF mutation.

109. The method of any one of claims 89-108, wherein the subject is a subject in whom it has been determined is homozygous for at least one of the one or more CFI mutations.

110. The method of any one of claims 89-108, wherein the subject is a subject in whom it has been determined is heterozygous for at least one of the one or more CFI mutations.

111. The vector or composition of any one of claims 1-41, wherein the vector or composition is capable of inducing at least 20%, 50%, 100%, 150%, 200%, 250%, 300%, 400%, 500%, 700%, 900%, 1000%, 1100%, 1500%, or 2000% expression of CFI in a target cell (e.g., an RPE or liver cell) as compared to the endogenous expression of CFI in the target cell.

112. The vector or composition of any one of claims 1-41, wherein the expression of the vector or composition in a target cell (e.g., an RPE or liver cell) results in at least 20%, 50%, 100%, 150%, 200%, 250%, 300%, 400%, 500%, 700%, 900%, 1000%, 1100%, 1500%, or 2000% levels of CFI activity in the target cell as compared to endogenous levels of CFI activity in the target cell.

113. The vector or composition of any one of claims 1-41, 111, or 112, wherein the vector or composition induces CFI expression in a target cell of the eye.

114. The vector or composition of claim 113, wherein the vector or composition induces CFI expression in a target cell of the retina or macula.

115. The vector or composition of claim 114, wherein the target cell of the retina is selected from the group of layers consisting of: inner limiting membrane, nerve fiber, ganglion cell layer (GCL), inner plexiform layer, inner nuclear layer, outer plexiform layer, outer nuclear layer, external limiting membrane, rods and cones, and retinal pigment epithelium (RPE).

116. The vector or composition of claim 113, wherein the target cell is in the choroid plexus.

117. The vector or composition of claim 114, wherein the target cell is in the macula.

118. The vector or composition of any one of claims 1-41 or 111-117, wherein the vector or composition induces CF expression in a cell of the GCL and/or RPE.

119. The vector or composition of any one of claims 1-41 or 111-118, wherein the CF is processed to an active CFI.

120. The vector or composition of any one of claims 1-41 or 111-119, wherein the vector comprises AAV.7m8.


SEQUENCE LISTING (35 sequences)

SEQ ID NO: 1 - DNA, 1823 bp, Artificial Sequence
  Description of Artificial Sequence Synthetic polynucleotide
gtccaggcggccgccaccatgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcagcagctagacatcctacaattcaaggctttgcatctgtggctcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataataagcttggatccagatctaatcaacctc

SEQ ID NO: 2 - DNA, 1807 bp, Artificial Sequence
  Description of Artificial Sequence Synthetic polynucleotide
gcggccgccaccatgaaactgctgcatgtctttctgctgtttctgtgcttccatctgcgcttctgcaaggtcacttacacttctcaggaggatctggtcgagaagaagtgtctggccaagaagtacacacacctgagctgcgacaaggtgttctgtcagccttggcagagatgcatcgagggcacctgcgtgtgcaagctgccttaccagtgcccaaagaacggaaccgccgtgtgcgcaacaaatcggcggagctttccaacatattgccagcagaagagcctggagtgtctgcaccccggcaccaagttcctgaacaatggcacctgcacagccgagggcaagttttctgtgagcctgaagcacggcaacacagatagcgagggcatcgtggaggtgaagctggtggaccaggataagaccatgttcatctgtaagagctcctggtccatgagggaggcaaacgtggcatgcctggatctgggattccagcagggagcagacacacagaggcgctttaagctgtccgacctgtctatcaatagcaccgagtgcctgcacgtgcactgtaggggcctggagacatccctggcagagtgcaccttcacaaagcggagaaccatgggctaccaggactttgccgacgtggtgtgctatacccagaaggccgatagcccaatggacgatttctttcagtgcgtgaacggcaagtatatctcccagatgaaggcctgcgacggcatcaatgactgtggcgatcagtctgacgagctgtgctgtaaggcctgtcagggcaagggcttccactgcaagagcggcgtgtgcatcccttcccagtaccagtgcaacggcgaggtggattgtatcacaggagaggacgaagtgggatgcgctgccgccagacacccaaccatccagggctttgcctctgtggcccaggaggagacagagatcctgacagccgacatggatgccgagaggcgccggatcaagtctctgctgcccaagctgagctgcggcgtgaagaataggatgcacatcagaaggaagcgcatcgtgggaggcaagagggcacagctgggcgatctgccttggcaggtggccatcaaggacgcctctggcatcacctgcggcggcatctacatcggaggatgttggatcctgaccgcagcacactgcctgagagcaagcaagacacacaggtatcagatttggaccacagtggtggattggatccacccagacctgaagagaatcgtgatcgagtacgtggataggatcatcttccacgagaactacaatgccggcacatatcagaacgacatcgccctgatcgagatgaagaaggatggcaataagaaggactgtgagctgccacgctccatccctgcatgcgtgccctggagcccctatctgttccagcccaacgatacctgtatcgtgtccggctggggccgcgagaaggacaatgagcgggtgttttctctgcagtggggcgaggtgaagctgatctccaactgttctaagttctacggcaatcggttttatgagaaggagatggagtgcgccggcacctacgatggcagcatcgacgcctgtaagggcgattccggaggaccactggtgtgcatggacgcaaacaatgtgacatacgtgtggggcgtggtgtcctggggcgagaattgcggcaagccagagtttcccggcgtgtataccaaggtggccaactattttgattggatttcctaccatgtcgggagaccattcatttcacagtataacgtgtaataagcttggatccagatct

SEQ ID NO: 3 - DNA, 1807 bp, Homo sapiens
gcggccgccaccatgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcagcagctagacatcctacaattcaaggctttgcatctgtggctcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataataagcttggatccagatct

SEQ ID NO: 4 - protein, 4 aa, Unknown
  Description of Unknown ′SFTL′ sequence
SFTL

SEQ ID NO: 5 - DNA, 1752 bp, Homo sapiens
atgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtgactcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataa

SEQ ID NO: 6 - DNA, 1644 bp, Artificial Sequence
  Description of Artificial Sequence Synthetic polynucleotide
acgcgtgttaactagtggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 7 - DNA, 6512 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttaactagtggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaaccggtctcgaaggcctgcaggcggccgccgccaccatgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtgactcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 8 - DNA, 432 bp, Artificial Sequence
  Description of Artificial Sequence Synthetic polynucleotide
acgcgttaactagtaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgaccatcgat

SEQ ID NO: 9 - DNA, 226 bp, Artificial Sequence
  Description of Artificial Sequence Synthetic polynucleotide
acgcgttaactagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 10 - DNA, 150 bp, Artificial Sequence
  Description of Artificial Sequence Synthetic polynucleotide
gtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgg

SEQ ID NO: 11 - DNA, 476 bp, Artificial Sequence
  Description of Artificial Sequence Synthetic polynucleotide
actagtcctgcagggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaacc

SEQ ID NO: 12 - DNA, 272 bp, Artificial Sequence
  Description of Artificial Sequence Synthetic polynucleotide
actagtccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggg

SEQ ID NO: 13 - DNA, 353 bp, Artificial Sequence
  Description of Artificial Sequence Synthetic polynucleotide
gttaacggctgcccactgggcatttcataggtggctcagtcctcttccctctgcagctggccccagaaacctgccagttattggtgccaggtctgtgccaggagggcgaggcctgtcatttctagtaatcctctgggcagtgtgactgtacctcttgcggcaactcaaagggagagggtgacttgtcccgggtcacagagctgaaagggcaggtacaacaggtgacatgccgggctgtctgagtttatgagggcccagtcttgtgtctgccgggcaatgagcaaggctccttcctgtccaagctccccgcccctccccagcctactgcctccacccgaagtctacttcctggg

SEQ ID NO: 14 - DNA, 5227 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttaacggctgcccactgggcatttcataggtggctcagtcctcttccctctgcagctggccccagaaacctgccagttattggtgccaggtctgtgccaggagggcgaggcctgtcatttctagtaatcctctgggcagtgtgactgtacctcttgcggcaactcaaagggagagggtgacttgtcccgggtcacagagctgaaagggcaggtacaacaggtgacatgccgggctgtctgagtttatgagggcccagtcttgtgtctgccgggcaatgagcaaggctccttcctgtccaagctccccgcccctccccagcctactgcctccacccgaagtctacttcctgggaccggtctcgaaggcctgcaggcggccgccgccaccatgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtgactcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 15 - DNA, 144 bp, Artificial Sequence
  Description of Artificial Sequence Synthetic polynucleotide
gttaaccagttccagatggtaaatatacacaagggatttagtcaaacaattttttggcaagaatattatgaattttgtaatcggttggcagccaatgaaatacaaagatgagtctagttaataatctacaattattggttaaag

SEQ ID NO: 16 - DNA, 5018 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttaaccagttccagatggtaaatatacacaagggatttagtcaaacaattttttggcaagaatattatgaattttgtaatcggttggcagccaatgaaatacaaagatgagtctagttaataatctacaattattggttaaagaccggtctcgaaggcctgcaggcggccgccgccaccatgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtgactcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 17 - DNA, 978 bp, Artificial Sequence
  Description of Artificial Sequence Synthetic polynucleotide
gttaacttggcaaagaattctgcagtcgacggtaccgcgggcccgggatccaccggtcgccaccatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatc

SEQ ID NO: 18 - DNA, 5852 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttaacttggcaaagaattctgcagtcgacggtaccgcgggcccgggatccaccggtcgccaccatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcaccggtctcgaaggcctgcaggcggccgccgccaccatgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtgactcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 19 - DNA, 1632 bp, Artificial Sequence
  Description of Artificial Sequence Synthetic polynucleotide
tagtggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 20 - DNA, 6512 bp, Artificial Sequence; identical to SEQ ID NO: 7
  Description of Artificial Sequence Synthetic polynucleotide

SEQ ID NO: 21 - DNA, 596 bp, Artificial Sequence
  Description of Artificial Sequence Synthetic polynucleotide
gttaacgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgacc

SEQ ID NO: 22 - DNA, 5470 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttaacgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgaccaccggtctcgaaggcctgcaggcggccgccgccaccatgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtgactcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 23 - DNA, 212 bp, Artificial Sequence
  Description of Artificial Sequence Synthetic polynucleotide
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 24 - DNA, 5092 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttaacgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagaccggtctcgaaggcctgcaggcggccgccgccaccatgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtgactcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 25 - DNA, 1585 bp, Artificial Sequence
  Description of Artificial Sequence Synthetic polynucleotide
gttaactatatttattgaagtttaatattgtgtttgtgatacagaagtatttgctttaattctaaataaaaattttatgcttttattgctggtttaagaagatttggattatccttgtactttgaggagaagtttcttatttgaaatattttggaaacaggtcttttaatgtggaaagatagatattaatctcctcttctattactctccaagatccaacaaaagtgattataccccccaaaatatgatggtagtatcttatactaccatcattttataggcatagggctcttagctgcaaataatggaactaactctaataaagcagaacgcaaatattgtaaatattagagagctaacaatctctgggatggctaaaggatggagcttggaggctacccagccagtaacaatattccgggctccactgttgaatggagacactacaactgccttggatgggcagagatattatggatgctaagccccaggtgctaccattaggacttctaccactgtccctaacgggtggagcccatcacatgcctatgccctcactgtaaggaaatgaagctactgttgtatatcttgggaagcacttggattaattgttatacagttttgttgaagaagacccctagggtaagtagccataactgcacactaaatttaaaattgttaatgagtttctcaaaaaaaatgttaaggttgttagctggtatagtatatatcttgcctgttttccaaggacttctttgggcagtaccttgtctgtgctggcaagcaactgagacttaatgaaagagtattggagatatgaatgaattgatgctgtatactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagttgg

SEQ ID NO: 26 - DNA, 6459 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttaactatatttattgaagtttaatattgtgtttgtgatacagaagtatttgctttaattctaaataaaaattttatgcttttattgctggtttaagaagatttggattatccttgtactttgaggagaagtttcttatttgaaatattttggaaacaggtcttttaatgtggaaagatagatattaatctcctcttctattactctccaagatccaacaaaagtgattataccccccaaaatatgatggtagtatcttatactaccatcattttataggcatagggctcttagctgcaaataatggaactaactctaataaagcagaacgcaaatattgtaaatattagagagctaacaatctctgggatggctaaaggatggagcttggaggctacccagccagtaacaatattccgggctccactgttgaatggagacactacaactgccttggatgggcagagatattatggatgctaagccccaggtgctaccattaggacttctaccactgtccctaacgggtggagcccatcacatgcctatgccctcactgtaaggaaatgaagctactgttgtatatcttgggaagcacttggattaattgttatacagttttgttgaagaagacccctagggtaagtagccataactgcacactaaatttaaaattgttaatgagtttctcaaaaaaaatgttaaggttgttagctggtatagtatatatcttgcctgttttccaaggacttctttgggcagtaccttgtctgtgctggcaagcaactgagacttaatgaaagagtattggagatatgaatgaattgatgctgtatactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagttggaccggtctcgaaggcctgcaggcggccgccgccaccatgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtgactcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 27 - DNA, 496 bp, Artificial Sequence
  Description of Artificial Sequence Synthetic polynucleotide
gttaacagcccccagttaggttaggcatttccaatctttgccaataagccacatatttgcccaagttagggtgcatccttcccatgaactttgactgtgacctttgactatggggtgacatcttatagctgtggtgttttgccaaccagcagctcttggtacacaaaatgtgctgctagcaggtgccccggccaaccttgtccttgacccacctgcctgttaagaaaagggtgttgtgttttgcaacagcagtaaaatgggtcaaggtttagtcagttggaagttgtgtcaaaactcactatggttggttgagggctcgaagtctcccagcattcattaacaactatctgttcaatgattatctccctggggcgtgttgcagtgagttggcccaaagcataactgaccctggccgtgatccagagacctgccccctgacgtcagtggcgagcctccctgggtgcagctgaggggcagggctattcttttccacagt

SEQ ID NO: 28 - DNA, 5370 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttaacagcccccagttaggttaggcatttccaatctttgccaataagccacatatttgcccaagttagggtgcatccttcccatgaactttgactgtgacctttgactatggggtgacatcttatagctgtggtgttttgccaaccagcagctcttggtacacaaaatgtgctgctagcaggtgccccggccaaccttgtccttgacccacctgcctgttaagaaaagggtgttgtgttttgcaacagcagtaaaatgggtcaaggtttagtcagttggaagttgtgtcaaaactcactatggttggttgagggctcgaagtctcccagcattcattaacaactatctgttcaatgattatctccctggggcgtgttgcagtgagttggcccaaagcataactgaccctggccgtgatccagagacctgccccctgacgtcagtggcgagcctccctgggtgcagctgaggggcagggctattcttttccacagtaccggtctcgaaggcctgcaggcggccgccgccaccatgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtgactcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 29 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 30 - protein, 1230 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAK

SEQ ID NO: 31 - protein, 449 aa, Unknown
  Description of Unknown FHL1 sequence
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 32 - DNA, 223 bp, Artificial Sequence
  Description of Artificial Sequence Synthetic polynucleotide
ggccgaaatggacaggaaatctcgccaattgacggcatcgccgctgagactcccccctcccccgtcctccccgtcccagcccggccatcacagccaatgacgggcgggctcgcagcggcgccgagggcggggcgcgggcgcgcaggtgcagcagcgcgcgggccggccaagagggcggggcgcgacgtcggccgtgcggggtcccggcgtcggcggcgcgcgc

SEQ ID NO: 33 - DNA, 6427 bp, Artificial Sequence
  Description of Artificial Sequence Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttaactagtggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaaccggtctcgaaggcctgcaggcggccgccgccaccatgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtggctcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 34 - DNA, 1752 bp, Homo sapiens
atgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtggctcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataa

SEQ ID NO: 35 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVAQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV